(19) United States
(12) Patent Application Publication
(10) Pub. No.: US 2022/0168433 A1
(43) Pub. Date: Jun. 2, 2022

(54) PROGRAMMABLE POLYMERIC DRUGS

(71) Applicant: SONY GROUP CORPORATION, Tokyo (JP)

(72) Inventors: Tracy MATRAY, Snohomish, WA (US); Sharat SINGH, Rancho Santa Fe, CA (US); C. Frederick BATTRELL, Wenatchee, WA (US); Michael VANBRUNT, Covington, WA (US)

(21) Appl. No.: 17/602,722

(22) PCT Filed: Apr. 10, 2020
(86) PCT No.: PCT/US2020/027755

§ 371 Date: Oct. 8, 2021

Related U.S. Application Data
(60) Provisional application No. 62/832,726, filed on Apr. 11, 2019.
(60) Provisional application No. 62/877,151, filed on Jul. 22, 2019.

(51) Int. Cl. A61K 47/58; A61K 47/68; A61K 49/00; C07F 9/572

(52) U.S. Cl. CPC A61K 47/605; A61K 47/6803; A61K 47/6849; A61K 47/6883; C07F 9/5728; A61K 49/0043; A61K 49/0054; A61K 49/0058; A61K 47/6889

(57) ABSTRACT

Compounds useful as biologically active compounds are disclosed. The compounds have the following structure (I): or a stereoisomer, tautomer or salt thereof, wherein R¹, R², R³, R⁴, R⁵, L^{a}, L^{b}, L¹, L², L³, M, m, and n are as defined herein. Methods associated with preparation and use of such compounds is also provided.

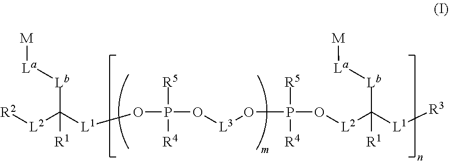

### BACKGROUND

### Field

Embodiments of the present disclosure are generally directed to dimeric and polymeric biologically active compounds having spacing groups, and methods for their preparation and use in various therapeutic methods.

### Description of the Related Art

Targeted drug conjugates, unlike, e.g., chemotherapy, deliver drugs to target cells, with little or no off-target activity. Typically, targeted drug conjugates are comprise a targeting molecule that is linked to a biologically active payload or drug. By combining the unique targeting capability with the therapeutic effectiveness of a biologically active drug, conjugates can deliver the drug only to the intended target and minimize potential side effects.

Antibody-drug conjugates (ADCs) are one class of targeted drug conjugates that are of particular interest, for example for cancer treatment. ADCs for cancer treatment combine the targeting features of monoclonal antibodies with cancer-killing ability of cytotoxic agents to provide a therapeutic with several advantages over other chemotherapeutics. However, challenges related to the complexity of ADC constructs, specifically the chemical linker between antibody and drug, has caused significant difficulties for development of new and effective therapeutics. Although the first ADC was approved in 2001, it took almost a decade before the next ADC was approved. As of today, only Adcetris®, Besponsa®, Enhertu®, Mylotarg®, Padcev®, Polivy®, and Kadcyla® are commercially available globally (Zevalin® has been approved in China only).

Thus, there exists a need in the art for potent, targeting drug conjugates having a high therapeutic index. The present disclosure fulfills this need and provides further related advantages.

### BRIEF SUMMARY

In brief, embodiments of the present disclosure are generally directed to compounds useful for delivery of biologically active moieties in vivo. Specific examples include targeted drug conjugates, optionally comprising fluorescent and/or colored dyes that enable selective delivery to targets, such as tumor cells. Methods and reagents for preparation of such molecules as well as use of the same for providing therapeutic treatment to a patient in need thereof are also described.

Embodiments of the presently disclosed compounds include one or more biologically active moieties covalently linked by linkers (e.g., “L^{a}” and/or “L^{b}”) to a common backbone. In addition, certain embodiments described herein provide compounds having multiple biologically active moieties within the same compound, and may further optionally include a targeting moiety. The biological moieties may either be the same or different, thus allowing for single agent or combination treatment by administration of a single compound.

In one embodiment, compounds having the following structure (I) are provided:

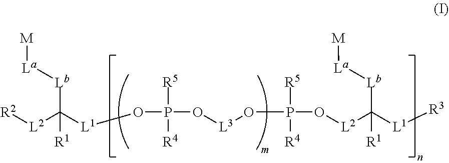

or a stereoisomer, tautomer or salt thereof, wherein R¹, R², R³, R⁴, R⁵, L^{a}, L^{b}, L¹, L², L³, M, m, and n are as defined herein. Compounds of structure (I) find utility in a number of applications, including use as therapeutic agents for various treatment methods.

In yet another embodiment compositions are provided which comprise a compound of structure (I) and a pharmaceutically acceptable carrier.

In another embodiment, a method of treating a disease is provided, the method comprising administering to a subject in need thereof a therapeutically effective amount of a compound of structure (I) or a composition comprising a compound of structure (I), wherein each M is independently a biologically active moiety effective for treating the disease.

These and other aspects of the disclosure will be apparent upon reference to the following detailed description.

### DETAILED DESCRIPTION

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Amino” refers to the —NH₂ group.

“Carboxy” refers to the —CO₂H group.

“Cyano” refers to the —CN group.

“Formyl” refers to the —C(═O)H group.

“Hydroxy” or “hydroxyl” refers to the —OH group.

“Imino” refers to the ═NH group.

“Nitro” refers to the —NO₂ group.

“Oxo” refers to the ═O group.

“Sulfhydryl,” “thiol” or “thio” refers to the —SH group.

“Thioxo” refers to the ═S group.

“Alkyl” refers to a straight or branched hydrocarbon chain group consisting solely of carbon and hydrogen atoms, containing no unsaturation, having from one to twelve carbon atoms (C₁-C₁₂ alkyl), one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, alkyl groups are optionally substituted.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, alkylene is optionally substituted.

“Alkenylene” or “alkenylene chain” refers to a straight or branched divalent hydrocarbon chain linking the rest of the molecule to a radical group, consisting solely of carbon and hydrogen, containing at least one carbon-carbon double bond and having from two to twelve carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain. Unless stated otherwise specifically in the specification, alkenylene is optionally substituted.

“Alkynylene” or “alkynylene chain” refers to a straight or branched divalent hydrocarbon chain linking the rest of the molecule to a radical group, consisting solely of carbon and hydrogen, containing at least one carbon-carbon triple bond and having from two to twelve carbon atoms, e.g., ethenylene, propenylene, n-butenylene, and the like. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain. Unless stated otherwise specifically in the specification, alkynylene is optionally substituted.

“Alkylether” refers to any alkyl group as defined above, wherein at least one carbon-carbon bond is replaced with a carbon-oxygen-carbon bond. The carbon-oxygen-carbon bond may be on the terminal end (as in an alkoxy group) or the carbon oxygen bond may be internal (i.e., C—O—C). Alkylethers include at least one carbon-oxygen-carbon bond, but may include more than one. For example, polyethylene glycol (PEG) is included within the meaning of alkylether. Unless stated otherwise specifically in the specification, an alkylether group is optionally substituted. For example, in some embodiments an alkylether is substituted with an alcohol or —OP(═Rₐ)(R_{b})R_{c}, wherein each of Rₐ, R_{b} and R_{c} is as defined for compounds of structure (I).

“Alkoxy” refers to a group of the formula —ORₐ where Rₐ is an alkyl group as defined above containing one to twelve carbon atoms. Unless stated otherwise specifically in the specification, an alkoxy group is optionally substituted.

“Alkoxyalkylether” refers to a group of the formula —ORₐR_{b} where Rₐ is an alkylene group as defined above containing one to twelve carbon atoms, and R_{b} is an alkylether group as defined herein. Unless stated otherwise specifically in the specification, an alkoxyalkylether group is optionally substituted, for example substituted with an alcohol or —OP(═Rₐ)(R_{b})R_{c}, wherein each of Rₐ, R_{b} and R_{c} is as defined for compounds of structure (I).

“Heteroalkyl” refers to an alkyl group, as defined above, comprising at least one heteroatom (e.g., Si, N, O, P or S) within the alkyl group or at a terminus of the alkyl group. In some embodiments, the heteroatom is within the alkyl group (i.e., the heteroalkyl comprises at least one carbon-[heteroatom]ₓ-carbon bond, where x is 1, 2 or 3). In other embodiments, the heteroatom is at a terminus of the alkyl group and thus serves to join the alkyl group to the remainder of the molecule (e.g., M1-H-A), where M1 is a portion of the molecule, H is a heteroatom and A is an alkyl group). Unless stated otherwise specifically in the specification, a heteroalkyl group is optionally substituted. Exemplary heteroalkyl groups include ethylene oxide (e.g., polyethylene oxide), optionally including phosphorous-oxygen bonds, such as phosphodiester bonds.

“Heteroalkoxy” refers to a group of the formula —ORₐ where Rₐ is a heteroalkyl group as defined above containing one to twelve carbon atoms. Unless stated otherwise specifically in the specification, a heteroalkoxy group is optionally substituted.

“Heteroalkylene” refers to an alkylene group, as defined above, comprising at least one heteroatom (e.g., Si, N, O, P or S) within the alkylene chain or at a terminus of the alkylene chain. In some embodiments, the heteroatom is within the alkylene chain (i.e., the heteroalkylene comprises at least one carbon-[heteroatom]-carbon bond, where x is 1, 2 or 3). In other embodiments, the heteroatom is at a terminus of the alkylene and thus serves to join the alkylene to the remainder of the molecule (e.g., M1-H-A-M2, where M1 and M2 are portions of the molecule, H is a heteroatom and A is an alkylene). Unless stated otherwise specifically in the specification, a heteroalkylene group is optionally substituted. Exemplary heteroalkylene groups include ethylene oxide (e.g., polyethylene oxide) and the “C,” “HEG,” and “PEG 1K” linking groups illustrated below:

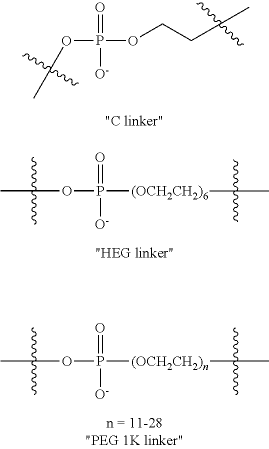

Multimers of the above C-linker, HEG linker and/or PEG 1K linker are included in various embodiments of heteroalkylene linkers. In some embodiments of the PEG 1K linker, n ranges from 19-25, for example n is 19, 20, 21, 22, 23, 24, or 25. Multimers may comprise, for example, the following structure:

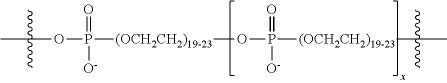

wherein x is 0 or an integer greater than 0, for example, x ranges from 0-100 (e.g., 1, 2, 3, 4, 5, 6, 7, 8, 9 or 10).

“Heteroalkenylene” is a heteroalkylene, as defined above, comprising at least one carbon-carbon double bond. Unless stated otherwise specifically in the specification, a heteroalkenylene group is optionally substituted.

“Heteroalkynylene” is a heteroalkylene comprising at least one carbon-carbon triple bond. Unless stated otherwise specifically in the specification, a heteroalkynylene group is optionally substituted.

“Heteroatomic” in reference to a “heteroatomic linker” refers to a linker group consisting of one or more heteroatoms. Exemplary heteroatomic linkers include single atoms selected from the group consisting of Si, O, N, P and S, and multiple heteroatoms for example a linker having the formula —P(O⁻)(═O)O— or —OP(O⁻)(═O)O— and multimers and combinations thereof.

“Phosphate” refers to the —OP(═O)(Rₐ)R_{b} group, wherein Rₐ is OH, O⁻ or OR_{c}; and R_{b} is OH, O⁻, OR_{c}, a thiophosphate group or a further phosphate group, wherein R_{c} is a counter ion (e.g., Na⁺ and the like).

“Phosphoalkyl” refers to the —OP(═O)(Rₐ)R_{b} group, wherein Rₐ is OH, O⁻ or OR_{c}; and R_{b} is —Oalkyl, wherein R_{c} is a counter ion (e.g., Na⁺ and the like). Unless stated otherwise specifically in the specification, a phosphoalkyl group is optionally substituted. For example, in certain embodiments, the —Oalkyl moiety in a phosphoalkyl group is optionally substituted with one or more of hydroxyl, amino, sulfhydryl, phosphate, thiophosphate, phosphoalkyl, thiophosphoalkyl, phosphoalkylether, thiophosphoalkylether or —OP(═Rₐ)(R_{b})R_{c}, wherein each of Rₐ, R_{b} and R_{c} is as defined for compounds of structure (I).

“Phosphoalkylether” refers to the —OP(═O)(Rₐ)R_{b} group, wherein Rₐ is OH, O⁻ or OR_{c}; and R_{b} is —Oalkylether, wherein R_{c} is a counter ion (e.g., Na⁺ and the like). Unless stated otherwise specifically in the specification, a phosphoalkylether group is optionally substituted. For example, in certain embodiments, the —Oalkylether moiety in a phosphoalkylether group is optionally substituted with one or more of hydroxyl, amino, sulfhydryl, phosphate, thiophosphate, phosphoalkyl, thiophosphoalkyl, phosphoalkylether, thiophosphoalkylether or —OP(═Rₐ)(R_{b})R_{c}, wherein each of Rₐ, R_{b} and R_{c} is as defined for compounds of structure (I).

“Thiophosphate” refers to the —OP(═Rₐ)(R_{b})R_{c} group, wherein Rₐ is O or S, R_{b} is OH, O⁻, S⁻, ORₐ or SR_{d}; and R_{c} is OH, SH, O⁻, S⁻, OR_{d}, SR_{d}, a phosphate group or a further thiophosphate group, wherein Rₐ is a counter ion (e.g., Na⁺ and the like) and provided that: i) Rₐ is S; ii) R_{b} is S⁻ or SR_{d}; iii) R_{c} is SH, S⁻ or SR_{d}; or iv) a combination of i), ii) and/or iii).

“Thiophosphoalkyl” refers to the —OP(═Rₐ)(R_{b})R_{c} group, wherein Rₐ is O or S, R_{b} is OH, O⁻, S⁻, ORₐ or SR_{d}; and R_{c} is —Oalkyl, wherein Rₐ is a counter ion (e.g., Na⁺ and the like) and provided that: i) Rₐ is S; ii) R_{b} is S⁻ or SR_{d}; or iii) Rₐ is S and R_{b} is S⁻ or SR_{d}. Unless stated otherwise specifically in the specification, a thiophosphoalkyl group is optionally substituted. For example, in certain embodiments, the —Oalkyl moiety in a thiophosphoalkyl group is optionally substituted with one or more of hydroxyl, amino, sulfhydryl, phosphate, thiophosphate, phosphoalkyl, thiophosphoalkyl, phosphoalkylether, thiophosphoalkylether or —OP(═Rₐ)(R_{b})R_{c}, wherein each of Rₐ, R_{b} and R_{c} is as defined for compounds of structure (I).

“Thiophosphoalkylether” refers to the —OP(═Rₐ)(R_{b})R_{c} group, wherein Rₐ is O or S, R_{b} is OH, O⁻, S⁻, ORₐ or SR_{d}; and R_{c} is —Oalkylether, wherein Rₐ is a counter ion (e.g., Na⁺ and the like) and provided that: i) Rₐ is S; ii) R_{b} is S⁻ or SR_{d}; or iii) Rₐ is S and R_{b} is S⁻ or SR_{d}. Unless stated otherwise specifically in the specification, a thiophosphoalkylether group is optionally substituted. For example, in certain embodiments, the —Oalkylether moiety in a thiophosphoalkyl group is optionally substituted with one or more of hydroxyl, amino, sulfhydryl, phosphate, thiophosphate, phosphoalkyl, thiophosphoalkyl, phosphoalkylether, thiophosphoalkylether or —OP(═Rₐ)(R_{b})R_{c}, wherein each of Rₐ, R_{b} and R_{c} is as defined for compounds of structure (I).

“Carbocyclic” refers to a stable 3- to 18-membered aromatic or non-aromatic ring comprising 3 to 18 carbon atoms. Unless stated otherwise specifically in the specification, a carbocyclic ring may be a monocyclic, bicyclic, tricyclic or tetracyclic ring system, which may include fused or bridged ring systems, and may be partially or fully saturated. Non-aromatic carbocyclyl radicals include cycloalkyl, while aromatic carbocyclyl radicals include aryl. Unless stated otherwise specifically in the specification, a carbocyclic group is optionally substituted.

“Cycloalkyl” refers to a stable non-aromatic monocyclic or polycyclic carbocyclic ring, which may include fused or bridged ring systems, having from three to fifteen carbon atoms, preferably having from three to ten carbon atoms, and which is saturated or unsaturated and attached to the rest of the molecule by a single bond. Monocyclic cyclocalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo-[2.2.1]heptanyl, and the like. Unless stated otherwise specifically in the specification, a cycloalkyl group is optionally substituted.

“Aryl” refers to a ring system comprising at least one carbocyclic aromatic ring. In some embodiments, an aryl comprises from 6 to 18 carbon atoms. The aryl ring may be a monocyclic, bicyclic, tricyclic or tetracyclic ring system, which may include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted.

“Heterocyclic” refers to a stable 3- to 18-membered aromatic or non-aromatic ring comprising one to twelve carbon atoms and from one to six heteroatoms selected from the group consisting of nitrogen, oxygen and sulfur. Unless stated otherwise specifically in the specification, the heterocyclic ring may be a monocyclic, bicyclic, tricyclic or tetracyclic ring system, which may include fused or bridged ring systems; and the nitrogen, carbon or sulfur atoms in the heterocyclic ring may be optionally oxidized; the nitrogen atom may be optionally quaternized; and the heterocyclic ring may be partially or fully saturated. Examples of aromatic heterocyclic rings are listed below in the definition of heteroaryls (i.e., heteroaryl being a subset of heterocyclic). Examples of non-aromatic heterocyclic rings include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, pyrazolopyrimidinyl, quinuclidinyl, thiazolidinyl, tetrahydrofuryl, trioxanyl, trithianyl, triazinanyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclic group is optionally substituted.

“Heteroaryl” refers to a 5- to 14-membered ring system comprising one to thirteen carbon atoms, one to six heteroatoms selected from the group consisting of nitrogen, oxygen and sulfur, and at least one aromatic ring. For purposes of certain embodiments of this disclosure, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzthiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, benzoxazolinonyl, benzimidazolthionyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, pteridinonyl, purinyl, pyrrolyl, pyrazolyl, pyridinyl, pyridinonyl, pyrazinyl, pyrimidinyl, pryrimidinonyl, pyridazinyl, pyrrolyl, pyrido[2,3-d]pyrimidinonyl, quinazolinyl, quinazolinonyl, quinoxalinyl, quinoxalinonyl, quinolinyl, isoquinolinyl, tetrahydroquinolinyl, thiazolyl, thiadiazolyl, thieno[3,2-d]pyrimidin-4-onyl, thieno[2,3-d]pyrimidin-4-onyl, triazolyl, tetrazolyl, triazinyl, and thiophenyl (i.e. thienyl). Unless stated otherwise specifically in the specification, a heteroaryl group is optionally substituted.

“Fused” refers to a ring system comprising at least two rings, wherein the two rings share at least one common ring atom, for example two common ring atoms. When the fused ring is a heterocyclyl ring or a heteroaryl ring, the common ring atom(s) may be carbon or nitrogen. Fused rings include bicyclic, tricyclic, tertracyclic, and the like.

The term “substituted” used herein means any of the above groups (e.g., alkyl, alkylene, alkenylene, alkynylene, heteroalkylene, heteroalkenylene, heteroalkynylene, alkoxy, alkylether, alkoxyalkylether, heteroalkyl, heteroalkoxy, phosphoalkyl, phosphoalkylether, thiophosphoalkyl, thiophosphoalkylether, carbocyclic, cycloalkyl, aryl, heterocyclic and/or heteroaryl) wherein at least one hydrogen atom (e.g., 1, 2, 3 or all hydrogen atoms) is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In some embodiments, the optional substituent is —OP(═Rₐ)(R_{b})R_{c}, wherein each of Rₐ, R_{b} and R_{c} is as defined for compounds of structure (I). In addition, each of the foregoing substituents may also be optionally substituted with one or more of the above substituents.

“Conjugation” or “bio-conjugation” refers to a chemical strategy for forming a stable covalent bond between two molecules. The term “bio-conjugation” is generally used when one of the molecules is a biomolecule (e.g., an antibody). The product or compound resulting from such a strategy is a conjugate, is conjugated, or a grammatically equivalent phrase.

“Fluorescent” refers to a molecule which is capable of absorbing light of a particular frequency and emitting light of a different frequency. Fluorescence is well-known to those of ordinary skill in the art.

“Colored” refers to a molecule which absorbs light within the colored spectrum (i.e., red, yellow, blue and the like).

A “linker” refers to a contiguous chain of at least one atom, such as carbon, oxygen, nitrogen, sulfur, phosphorous and combinations thereof, which connects a portion of a molecule to another portion of the same molecule or to a different molecule, moiety or solid support (e.g., microparticle). Linkers may connect the molecule via a covalent bond or other means, such as ionic or hydrogen bond interactions.

The term “biomolecule” refers to any of a variety of biological materials, including nucleic acids, carbohydrates, amino acids, polypeptides, glycoproteins, hormones, aptamers and mixtures thereof. More specifically, the term is intended to include, without limitation, RNA, DNA, oligonucleotides, modified or derivatized nucleotides, enzymes, receptors, prions, receptor ligands (including hormones), antibodies, antigens, and toxins, as well as bacteria, viruses, blood cells, and tissue cells. In some embodiments of the disclosure, exemplary conjugates (e.g., compounds of structure (I) having a biomolecule linked thereto) are prepared, as further described herein, by contacting a biomolecule with a compound having a reactive group that enables attachment of the biomolecule to the compound via any available atom or functional group, such as an amino, hydroxy, carboxyl, or sulfhydryl group on the biomolecule.

A “reactive group” is a moiety capable of reacting with a second reactive group (e.g., a “complementary reactive group”) to form one or more covalent bonds, for example by a displacement, oxidation, reduction, addition or cycloaddition reaction. Exemplary reactive groups are provided in Table 1, and include for example, nucleophiles, electrophiles, dienes, dienophiles, aldehyde, oxime, hydrazone, alkyne, amine, azide, acylazide, acylhalide, nitrile, nitrone, sulfhydryl, disulfide, sulfonyl halide, isothiocyanate, imidoester, activated ester, ketone, α,β-unsaturated carbonyl, alkene, maleimide, α-haloamide, epoxide, aziridine, tetrazine, tetrazole, phosphine, biotin, thiirane and the like.

“Solid support” refers to any solid substrate known in the art for solid-phase support of molecules, for example a “microparticle” refers to any of a number of small particles useful for attachment to compounds of the disclosure, including, but not limited to, glass beads, magnetic beads, polymeric beads, nonpolymeric beads, and the like. In certain embodiments, a microparticle comprises polystyrene beads.

A “solid support residue” refers to the functional group remaining attached to a molecule when the molecule is cleaved from the solid support. Solid support residues are known in the art and can be easily derived based on the structure of the solid support and the group linking the molecule thereto.

A “targeting moiety” is a moiety that selectively binds or associates with a particular target, such as a tumor cell antigen. “Selectively” binding or associating means a targeting moiety preferentially associates or binds with the desired target relative to other targets. For example, selectively binding, in some embodiments, means a targeting moiety, or a conjugate comprising the same, that associates or binds to the desired target at least 10 times or at least 100 times greater relative to other targets. In some embodiments the compounds disclosed herein include linkages to targeting moieties for the purpose of selectively binding or associating the compound with a desired target, such as a tumor cell antigen, thus allowing targeted delivery of the biologically active moiety. Exemplary targeting moieties include, but are not limited to, antibodies, antigens, nucleic acid sequences, enzymes, proteins, cell surface receptor antagonists or cell surface receptor agonists, and the like. In some embodiments, the targeting moiety is a moiety, such as an antibody, that selectively binds or associates with a target feature on or in a cell, for example a target feature on a cell membrane or other cellular structure, thus allowing for delivery of a biologically active moiety to or into cells of interest. Small molecules that selectively bind or associate with a desired biological target are also contemplated as targeting moieties in certain embodiments. One of skill in the art will understand other biological targets, and the corresponding targeting moiety, that will be useful in various embodiments.

“Physiologically cleavable linker” refers to a molecular linkage that can be split or separated in a prescribed manner, resulting in two or more separate molecules while in the presence of an in vivo or in vitro environment of an organism or cell system. Generally, physiological conditions that induce such a cleavage or scission event may include a temperature ranging from about 20 to 40° C., an atmospheric pressure of about 1 atm (101 kPa or 14.7 psi), a pH of about 6 to 8, a glucose concentration of about 1 to 20 mM, atmospheric oxygen concentration, and earth gravity. In some embodiments, physiological conditions include enzymatic conditions (i.e., enzymatic cleavage). Bond cleavage or scission can be homolytic or heterolytic. Embodiments disclosed herein are also meant to encompass all compounds of structure (I) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively.

Isotopically-labeled compounds of structure (I) can generally be prepared by conventional techniques known to those skilled in the art or by processes analogous to those described below and in the following Examples using an appropriate isotopically-labeled reagent in place of the non-labeled reagent previously employed.

“Stable compound” and “stable structure” are meant to indicate a compound that is sufficiently robust to survive isolation to a useful degree of purity from a reaction mixture, and formulation into an efficacious therapeutic agent.

“Optional” or “optionally” means that the subsequently described event or circumstances may or may not occur, and that the description includes instances where said event or circumstance occurs and instances in which it does not. For example, “optionally substituted alkyl” means that the alkyl group may or may not be substituted and that the description includes both substituted alkyl groups and alkyl groups having no substitution.

“Salt” includes both acid and base addition salts.

“Acid addition salt” refers to those salts which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Base addition salt” refers to those salts which are prepared from addition of an inorganic base or an organic base to the free acid. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Crystallizations may produce a solvate of the compounds described herein. Embodiments of the present disclosure include all solvates of the described compounds. As used herein, the term “solvate” refers to an aggregate that comprises one or more molecules of a compound of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. The compounds of the disclosure may be true solvates, while in other cases the compounds of the disclosure may merely retain adventitious water or another solvent or be a mixture of water plus some adventitious solvent.

Embodiments of the compounds of the disclosure (e.g., compounds of structure I), or their salts, tautomers or solvates may contain one or more stereocenters and may thus give rise to enantiomers, diastereomers, and other stereoisomeric forms that may be defined, in terms of absolute stereochemistry, as (R)- or (S)- or, as (D)- or (L)- for amino acids. Embodiments of the present disclosure are meant to include all such possible isomers, as well as their racemic and optically pure forms. Optically active (+) and (−), (R)- and (5)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

When the compounds described herein contain olefinic double bonds or other features giving rise to geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

A “tautomer” refers to a proton shift from one atom of a molecule to another atom of the same molecule. The present disclosure includes tautomers of any said compounds. Various tautomeric forms of the compounds are easily derivable by those of ordinary skill in the art.

The chemical naming protocol and structure diagrams used herein are a modified form of the I.U.P.A.C. nomenclature system, using the ACD/Name Version 9.07 software program and/or ChemDraw Ultra Version 11.0 software naming program (CambridgeSoft). Common names familiar to one of ordinary skill in the art are also used.

As noted above, in one embodiment of the present disclosure, compounds comprising covalent linkers between one or more biologically active moieties and optional targeting moieties are provided. In other embodiments, compounds useful as synthetic intermediates for preparation of compounds comprising one or more biologically active moieties and optional targeting moieties are provided. In general terms, embodiments of the present disclosure are directed to polymers with pendant biologically active moieties. The biologically active moieties are linked to the polymer by a linking moiety. In another aspect, the linker provides a link between the biologically active moiety and a targeting moiety, which acts to increase accumulation of the biologically active moiety at the desired target. That is, the biological activity may be increased due to accumulation at the intended target, while off-target effects are reduced, thus minimizing potential side effects of the therapeutic (e.g., cytotoxicity).

In other embodiments is provided a compound having the following structure (I):

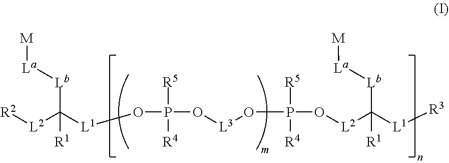

or a stereoisomer, pharmaceutically acceptable salt or tautomer thereof, wherein:

M is, at each occurrence, independently a biologically active moiety, or fragment thereof, a prodrug of a biologically active moiety, or fragment thereof, a fluorescent dye, an imaging agent, or a radioisotope binding site, provided at least one occurrence of M is not a fluorescent dye;

L^{a} is, at each occurrence, independently an optional physiologically cleavable linker and L^{b} is, at each occurrence, independently an optional physiologically non-cleavable linker, provided that at least one occurrence of L^{a} and L^{b} together

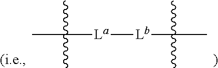

comprise more than 4 carbons;

L¹ and L² are, at each occurrence, independently an optional alkylene, alkenylene, alkynylene, heteroalkylene, heteroalkenylene, heteroalkynylene or heteroatomic linker;

L³ is, at each occurrence, independently an alkylene, alkenylene, alkynylene, heteroalkylene, heteroalkenylene or heteroalkynylene linker;

R¹ is, at each occurrence, independently H, alkyl or alkoxy;

R² and R³ are each independently H, OH, SH, alkyl, alkoxy, alkylether, heteroalkyl, —OP(═Rₐ)(R_{b})R_{c}, Q, or a protected form thereof, or L′;

R⁴ is, at each occurrence, independently O⁻, S⁻, OZ, SZ or N(R⁶)₂, where Z is a cation and each R⁶ is independently H or alkyl;

R⁵ is, at each occurrence, independently oxo, thioxo or absent; Rₐ is O or S;

R_{b} is OH, SH, O⁻, S⁻, ORₐ or SR_{d};

R_{c} is OH, SH, O⁻, S⁻, ORₐ, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;

R_{d} is a counter ion;

Q is, at each occurrence, independently a moiety comprising a reactive group, or protected form thereof, capable of forming a covalent bond with a complementary reactive group Q′ on a targeting moiety;

L′ is, at each occurrence, independently a linker comprising a covalent bond to Q, a targeting moiety, a linker comprising a covalent bond to a targeting moiety, a linker comprising a covalent bond to a solid support, a linker comprising a covalent bond to a solid support residue, a linker comprising a covalent bond to a nucleoside or a linker comprising a covalent bond to a further compound of structure (I);

m is, at each occurrence, independently an integer of zero or greater; and

n is an integer of one or greater.

In other embodiments is provided a compound having the following structure (I):

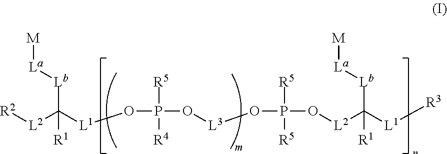

or a stereoisomer, pharmaceutically acceptable salt or tautomer thereof, wherein:

M is, at each occurrence, independently a biologically active moiety, or fragment thereof, a prodrug of a biologically active moiety, or fragment thereof, a fluorescent dye, an imaging agent, or a radioisotope binding site, provided at least one occurrence of M is not a fluorescent dye;

L^{a} is, at each occurrence, independently an optional physiologically cleavable linker and L^{b} is, at each occurrence, independently an optional physiologically non-cleavable linker provided that at least one occurrence of L^{a} and L^{b} together

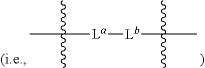

comprises carbon, oxygen, and nitrogen;

L¹ and L² are, at each occurrence, independently an optional alkylene, alkenylene, alkynylene, or heteroatomic linker;

L³ is, at each occurrence, independently an alkylene, alkenylene, alkynylene, heteroalkylene, heteroalkenylene or heteroalkynylene linker;

R¹ is, at each occurrence, independently H, alkyl or alkoxy;

R² and R³ are each independently H, OH, SH, alkyl, alkoxy, alkylether, heteroalkyl, —OP(═Rₐ)(R_{b})R_{c}, Q, or a protected form thereof, or L′;

R⁴ is, at each occurrence, independently O⁻, S⁻, OZ, SZ or N(R⁶)₂, where Z is a cation and each R⁶ is independently H or alkyl;

R⁵ is, at each occurrence, independently oxo, thioxo or absent;

Rₐ is O or S;

R_{b} is OH, SH, O⁻, S⁻, ORₐ or SR_{d};

R_{c} is OH, SH, O⁻, S⁻, ORₐ, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;

R_{d} is a counter ion;

Q is, at each occurrence, independently a moiety comprising a reactive group, or protected form thereof, capable of forming a covalent bond with a complementary reactive group Q′ on a targeting moiety;

L′ is, at each occurrence, independently a linker comprising a covalent bond to Q, a targeting moiety, a linker comprising a covalent bond to a targeting moiety, a linker comprising a covalent bond to a solid support, a linker comprising a covalent bond to a solid support residue, a linker comprising a covalent bond to a nucleoside or a linker comprising a covalent bond to a further compound of structure (I);

m is, at each occurrence, independently an integer of zero or greater; and

n is an integer of one or greater.

In some embodiments, at least one occurrence of L^{a} and L^{b} together

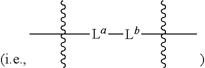

does not form following structure:

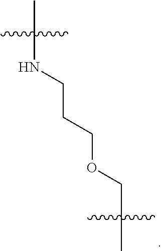 [SMILES: *COCCCN*]

The linkers L^{a} and/or L^{b} can be used as a point of attachment of the M moiety to the remainder of the compound. For example, in some embodiments a synthetic precursor to the compound of structure (I) is prepared, and the M moiety is attached to the synthetic precursor using any number of facile methods known in the art, for example methods referred to as “click chemistry.” For this purpose any reaction which is rapid and substantially irreversible can be used to attach M to the synthetic precursor to form a compound of structure (I). Exemplary reactions include the copper catalyzed reaction of an azide and alkyne to form a triazole (Huisgen 1,3-dipolar cycloaddition), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse-demand Diels-Alder, alkene and tetrazole photoreaction and various displacement reactions, such as displacement of a leaving group by nucleophilic attack on an electrophilic atom. Exemplary displacement reactions include reaction of an amine with: an activated ester; an N-hydroxysuccinimide ester; an isocyanate; an isothioscyanate or the like. In some embodiments the reaction to form L^{a} and/or L^{b} may be performed in an aqueous environment.

Accordingly, in some embodiments L^{a} and/or L^{b} is at each occurrence a linker comprising a functional group capable of formation by reaction of two complementary reactive groups, for example a functional group which is the product of one of the foregoing “click” reactions. In various embodiments, for at least one occurrence of L^{a} and/or L^{b}, the functional group can be formed by reaction of an aldehyde, oxime, hydrazone, alkyne, amine, azide, acylazide, acylhalide, nitrile, nitrone, sulfhydryl, disulfide, sulfonyl halide, isothiocyanate, imidoester, activated ester (e.g., N-hydroxysuccinimide ester), ketone, α,β-unsaturated carbonyl, alkene, maleimide, α-haloamide, epoxide, aziridine, tetrazine, tetrazole, phosphine, biotin or thiirane functional group with a complementary reactive group, for example, reaction of an amine with an N-hydroxysuccinimide ester or isothiocyanate.

In other embodiments, for at least one occurrence of L^{a} and/or L^{b}, the functional group can be formed by reaction of an alkyne and an azide. In other embodiments, for at least one occurrence of L^{a} and/or L^{b}, the functional group can be formed by reaction of an amine (e.g., primary amine) and an N-hydroxysuccinimide ester or isothiocyanate.

In more embodiments, for at least one occurrence of L^{a} and/or L^{b}, the functional group comprises an alkene, ester, amide, thioester, disulfide, carbocyclic, heterocyclic or heteroaryl group. In more embodiments, for at least one occurrence of L^{a} and/or L^{b}, the functional group comprises an alkene, ester, amide, thioester, thiourea, disulfide, carbocyclic, heterocyclic or heteroaryl group. In other embodiments, the functional group comprises an amide or thiourea. In some more specific embodiments, for at least one occurrence of L^{a} and/or L^{b}, L^{a} and/or L^{b} is a linker comprising a triazolyl functional group. While in other embodiments, for at least one occurrence of L^{a} and/or L^{b}, L^{a} and/or L^{b} is a linker comprising an amide or thiourea functional group.

Some embodiments provide an L^{a} that is able to be cleaved under the appropriate conditions (e.g., physiological conditions). In certain embodiments, at least one occurrence of L^{a} is present. In some more specific embodiments, at least one occurrence of L^{a} comprises an amide bond, an ester bond, a phosphodiester bond, a disulfide bond, a double bond, a triple bond, an ether bond, a hydrazone, an amino acid sequence, a ketone, a diol, a cyano, a nitro or combinations thereof.

In some embodiments, L^{a} comprises an amino acid sequence recognized by a sortase enzyme. In certain embodiments, the amino acid sequence is Leu-Pro-X-Thr-Gly, wherein X is any amino acid residue. In some other embodiments, at least one occurrence of L^{a} is a linker comprising 3 or more carbons. In certain other embodiments, at least one occurrence of L^{a} is a linker comprising at least one nitrogen. In some embodiments, at least one occurrence of L^{a} comprises one of the following structures:

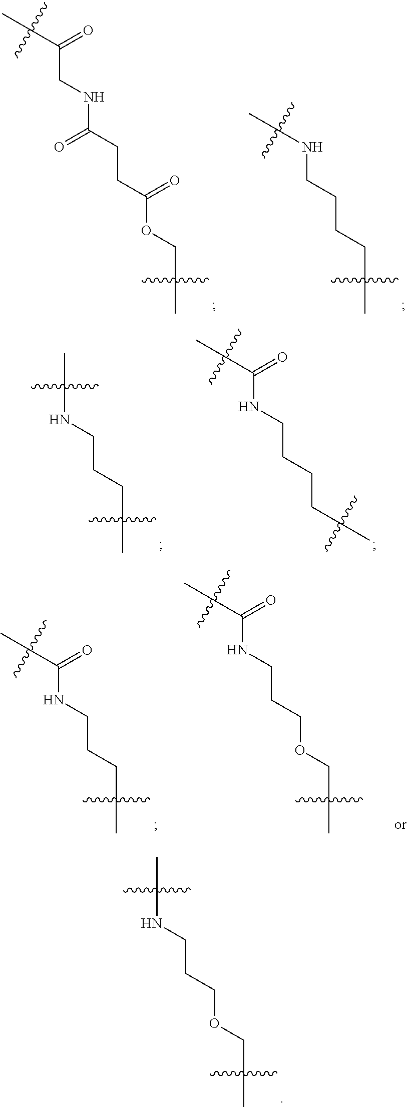

In some embodiments, each occurrence of L^{a} comprises an amide bond, an ester bond, a phosphodiester bond, a disulfide bond, a double bond, a triple bond, an ether bond, a hydrazone, an amino acid sequence, a ketone, a diol, a cyano, a nitro or combinations thereof.

In some embodiments, each occurrence of L^{a} is a linker comprising 3 or more carbons. In certain embodiments, each occurrence of L^{a} is a linker comprising at least one nitrogen. In some other embodiments, each occurrence of L^{a} comprises one of the following structures:

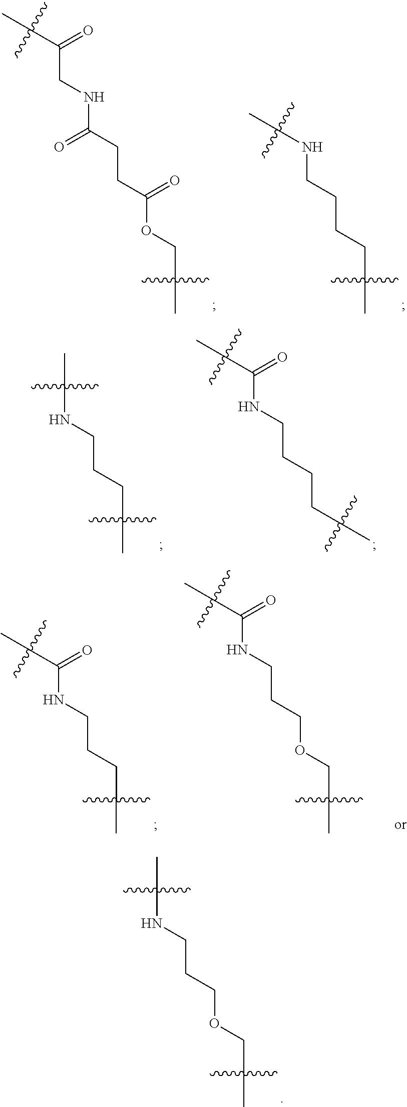

In some embodiments, at least one occurrence of L^{a} comprises the following structure:

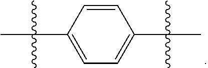 [SMILES: *c1ccc(*)cc1]

In certain embodiments, at least one occurrence of L^{a} comprises one or more amino acid residues. In some embodiments, the amino acid residue is valine. In certain more specific embodiments, at least one occurrence of L^{a} comprises one of the following structures:

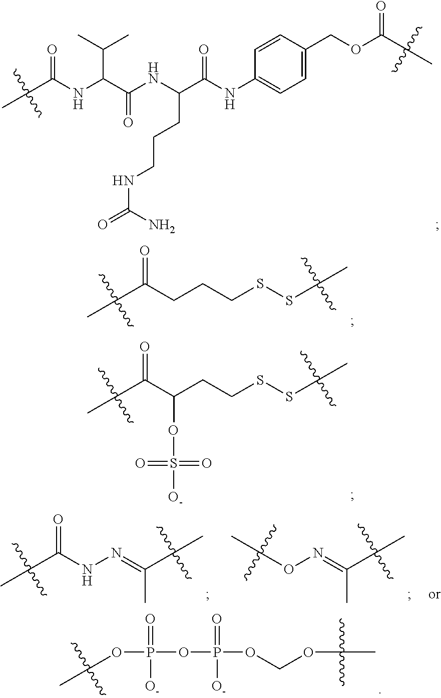

In some embodiments of structure (I), L^{a} comprises one of the following structures:

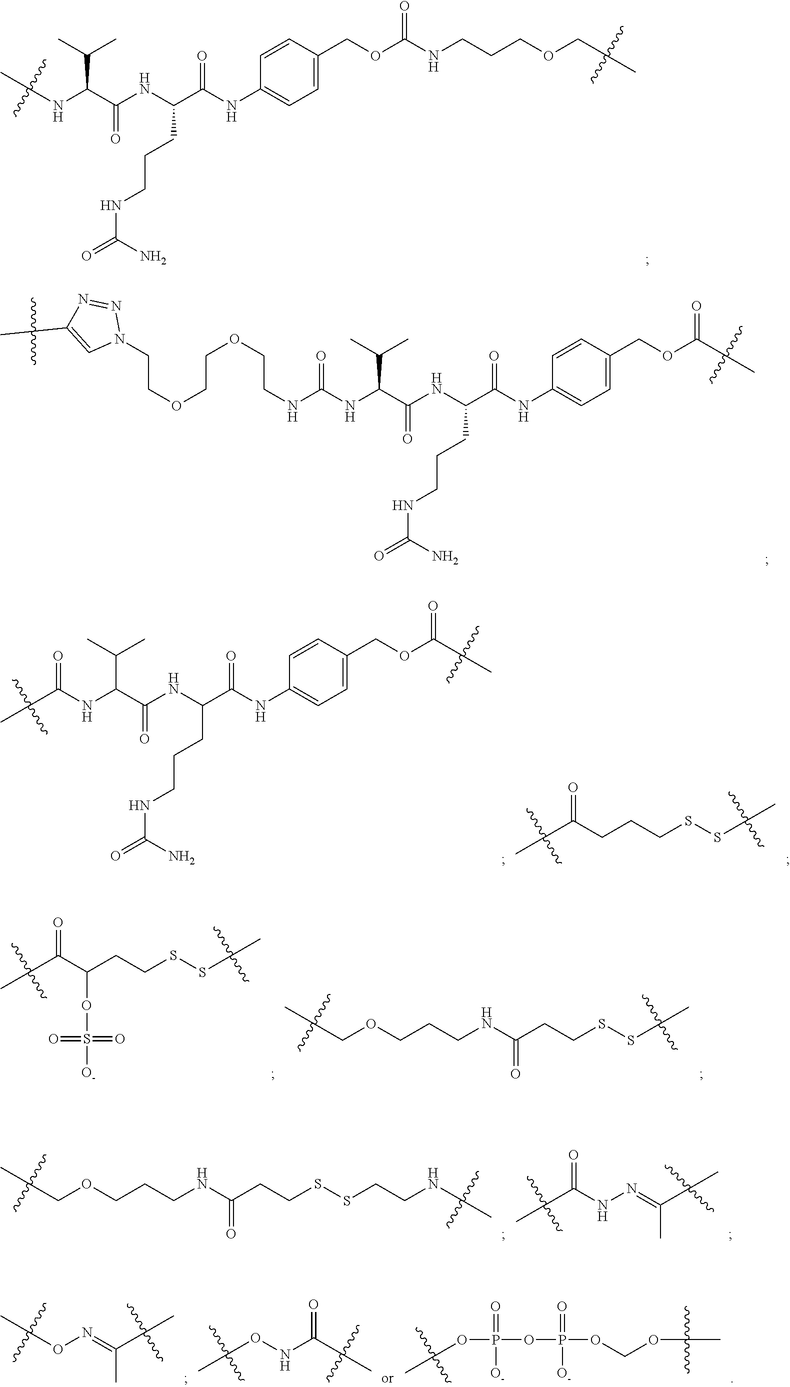

In some more specific embodiments, at least one occurrence of L^{a} comprises the following structure:

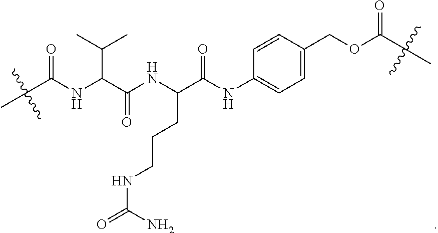 [SMILES: *C(=O)NC(C(=O)NC(CCCNC(N)=O)C(=O)Nc1ccc(COC(*)=O)cc1)C(C)C]

In some more specific embodiments, at least one occurrence of L^{a} comprises one of the following structures:

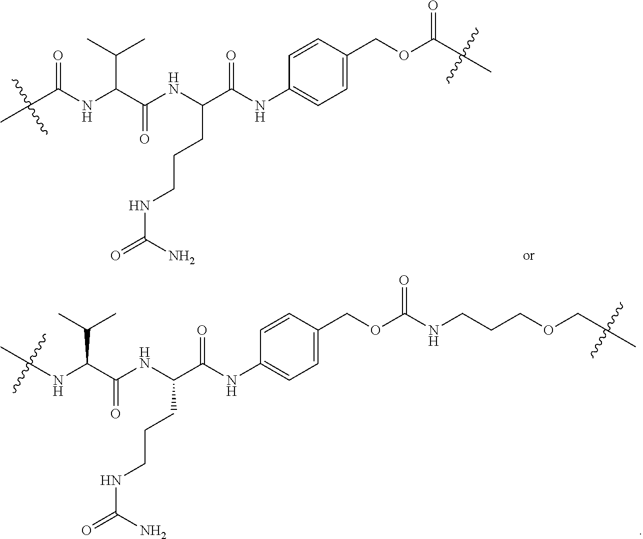 [SMILES: *C(=O)NC(C(=O)NC(CCCNC(N)=O)C(=O)Nc1ccc(COC(*)=O)cc1)C(C)C ; *COCCCNC(=O)OCc1ccc(NC(=O)[C@H](CCCNC(N)=O)NC(=O)[C@@H](N*)C(C)C)cc1]

In some embodiments, each occurrence of L^{a} comprises the following structure:

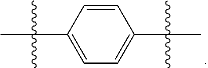 [SMILES: *c1ccc(*)cc1]

In some embodiments, each occurrence of L^{a} comprises one or more amino acid residues. In certain embodiments, the amino acid residue is valine. In certain specific embodiments, each occurrence of L^{a} comprises one of the following structures:

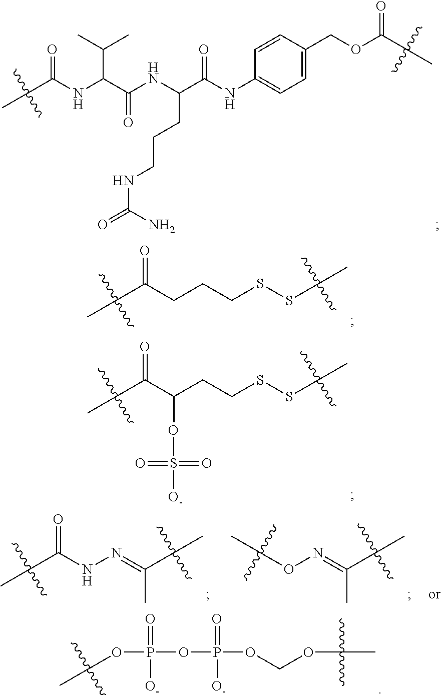

In some more specific embodiments, at least one occurrence of L^{a} comprises the following structure:

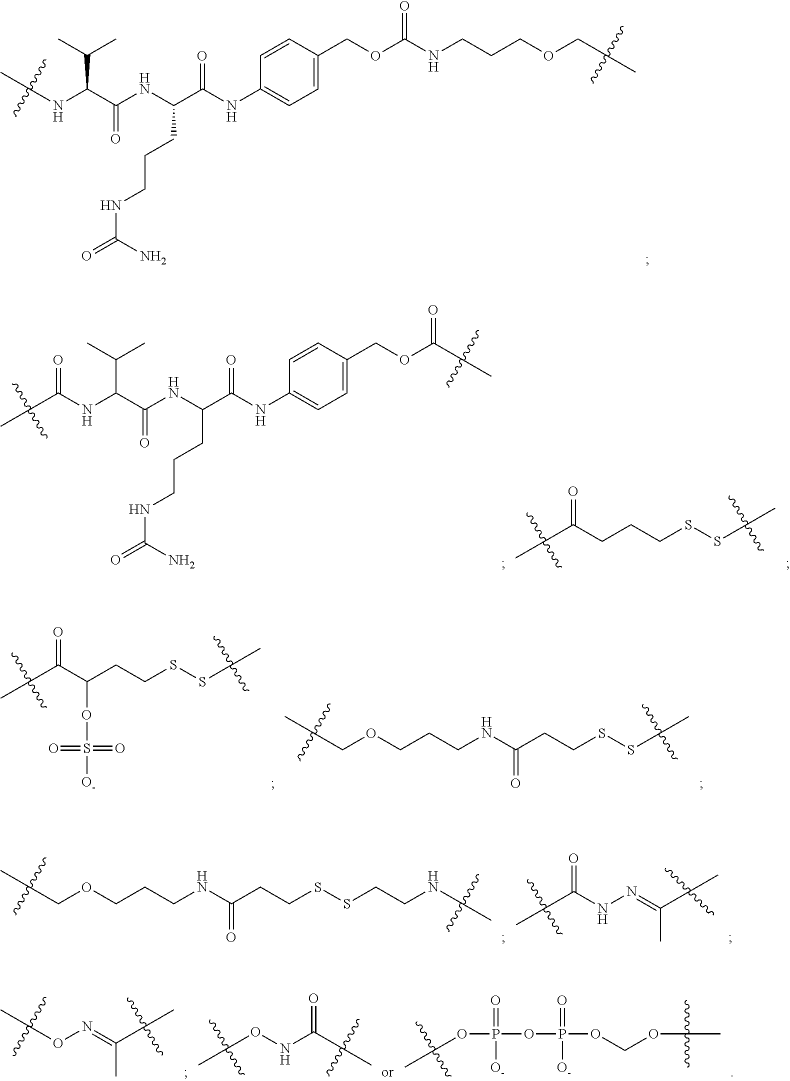

In some embodiments, at least one occurrence of L^{a} has the following structure:

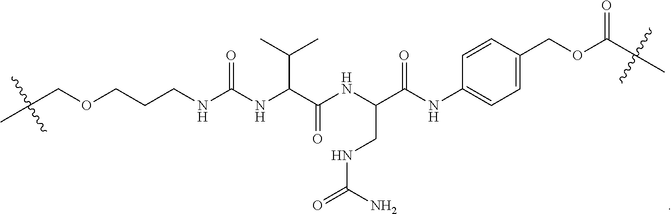 [SMILES: *COCCCNC(=O)NC(C(=O)NC(CNC(N)=O)C(=O)Nc1ccc(COC(*)=O)cc1)C(C)C]

In some embodiments, at least one occurrence of L^{a} has one of the following structures:

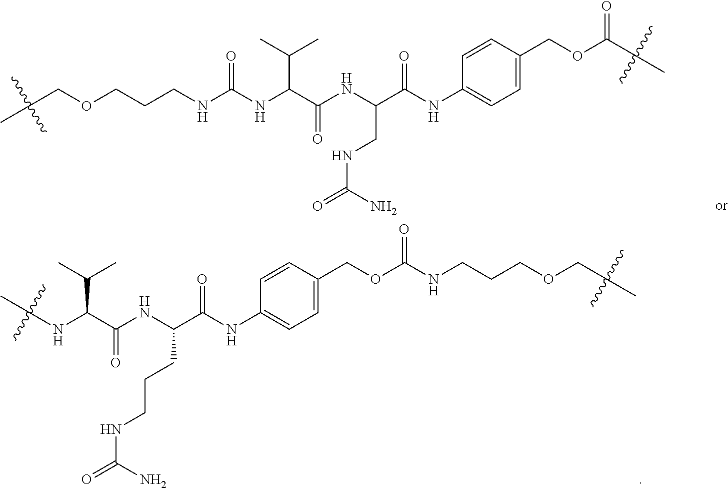

In certain embodiments, at least one occurrence of L^{a} has the following structure:

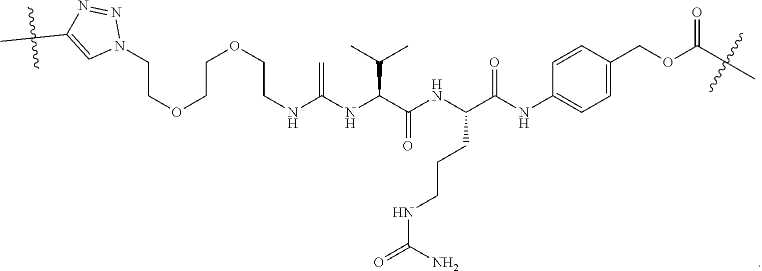 [SMILES: *C(=O)OCc1ccc(NC(=O)[C@H](CCCNC(N)=O)NC(=O)[C@@H](NC(=C)NCCOCCOCCn2cc(*)nn2)C(C)C)cc1]

In some embodiments, at least one occurrence of L^{b} is present (and comprises a linker that is non-cleavable under physiological conditions). In some specific embodiments, at least one occurrence of L^{b} comprises a thioether bond. In certain specific embodiments, at least one occurrence of L^{b} comprises the following structure:

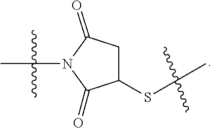

In some embodiments, at least one occurrence of L^{b} comprises one of the following structures:

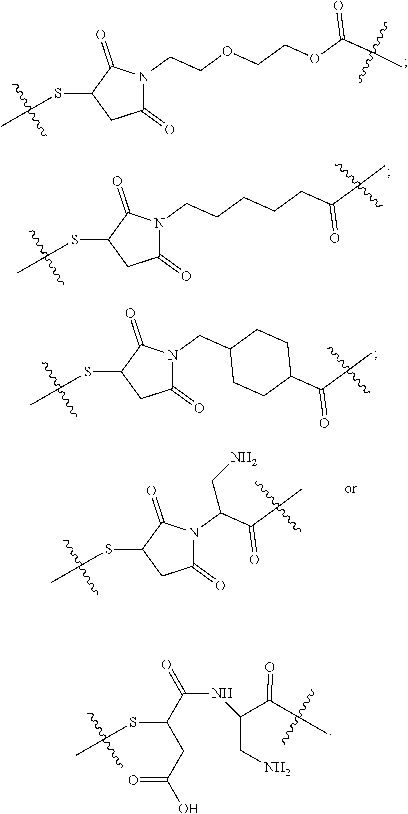

In some embodiments, each occurrence of L^{b} comprises a linker that is non-cleavable under physiological conditions. In certain embodiments, each occurrence of L^{b} comprises a thioether bond. In certain embodiments, each occurrence of L^{b} comprises the following structure:

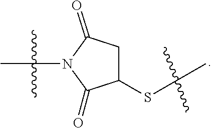

In some embodiments, each occurrence of L^{b} comprises one of the following structures:

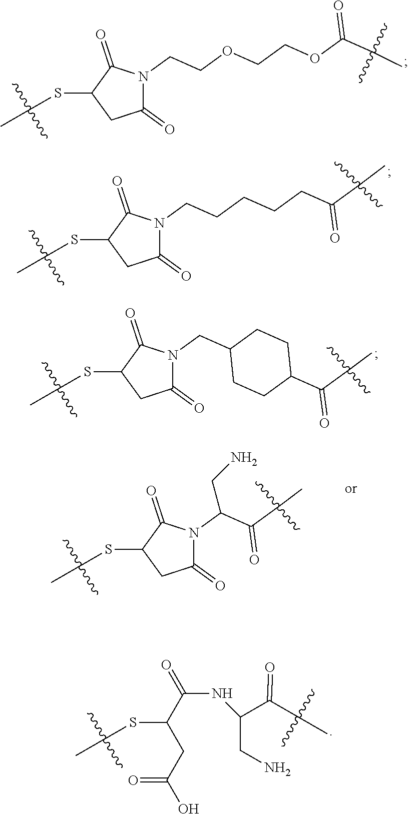

Accordingly, in some embodiments, L^{a} or L^{b} comprise an amide bond, an ester bond, a disulfide bond, a hydrazone, a phosphotriester, a diester, β-glucuronide, a double bond, a triple bond, an ether bond, a ketone, a diol, a cyano, a nitro or combinations thereof.

In some embodiments, L^{a} or L^{b} together comprise tert-butyloxycarbonyl, paramethoxybenzyl, dialkyl or diaryldialkoxysilane, orthoester, acetal, β-thiopropionate, ketal, phosphoramidate, hydrazone, vinyl ether, imine, aconityl, trityl, polyketal, bisarylhydrazone, diazobenzene, vivinal diol, pyrophosphate diester, or valine citrulline.

In certain embodiments, L^{a} is, at each occurrence, independently a linker that is cleavable at a pH ranging from 6 to 8. For example, in some embodiments L is a linker that is cleavable at pH 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

In certain embodiments, L^{a} is, at each occurrence, independently a linker that is cleavable at a temperature ranging from 20° C. to 40° C., from 25° C. to 35° C., from 30° C. to 35° C., from 30° C. to 37° C., from 35° C. to 37° C., from 35° C. to 40° C., from 32° C. to 38° C. In certain embodiments, L is, at each occurrence, independently a linker that is cleavable at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

In certain embodiments, L^{a} is, at each occurrence, independently a linker that is cleavable by an enzyme. For example, in some embodiments, the enzyme is a hydrolase, an oxidoreductase or a lyase. In certain embodiments, the enzyme is an EC 4.1 (e.g., EC 4.1.1, EC 4.1.2, EC 4.1.3 or EC 4.1.99), EC 4.2, EC 4.3, EC4.4, EC 4.5, EC 4.6, or EC 4.99 enzyme.

In certain embodiments, L^{a} comprises one of the following structures:

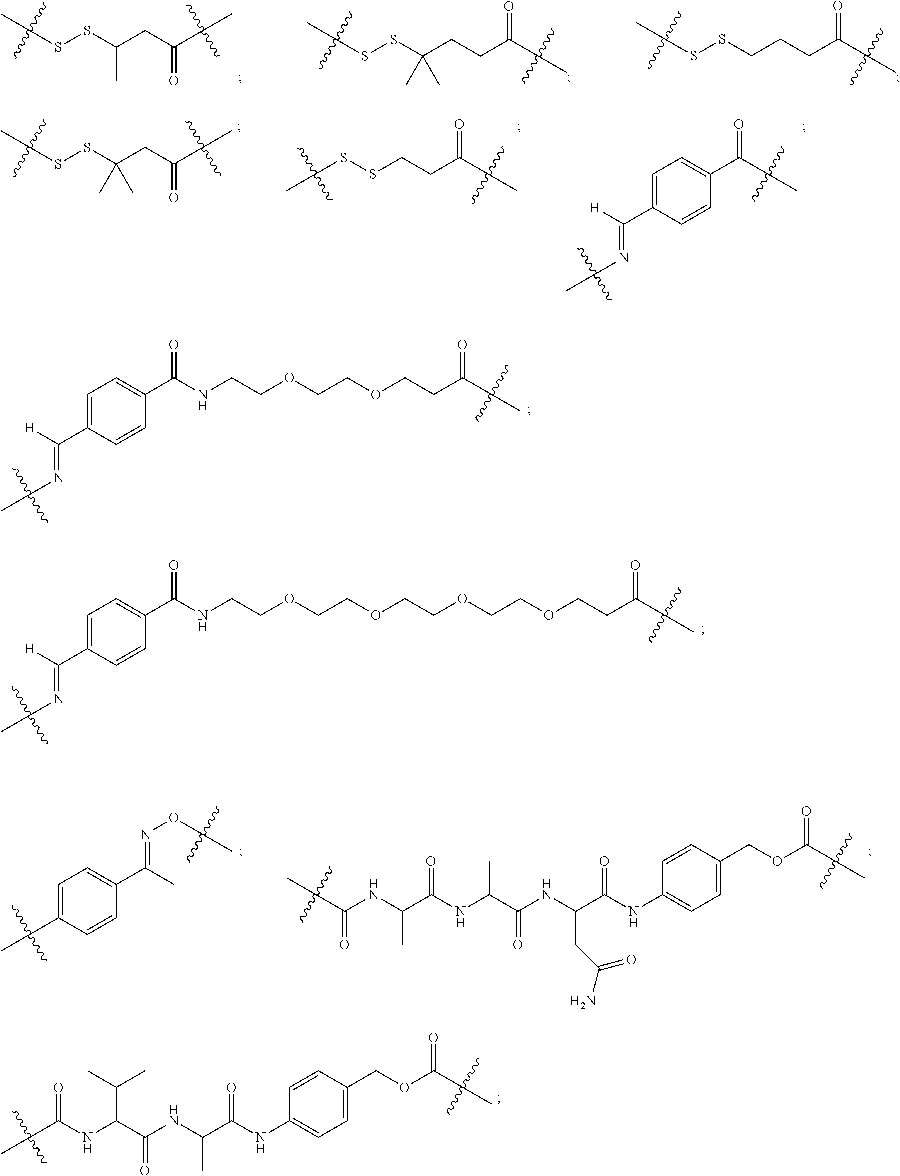

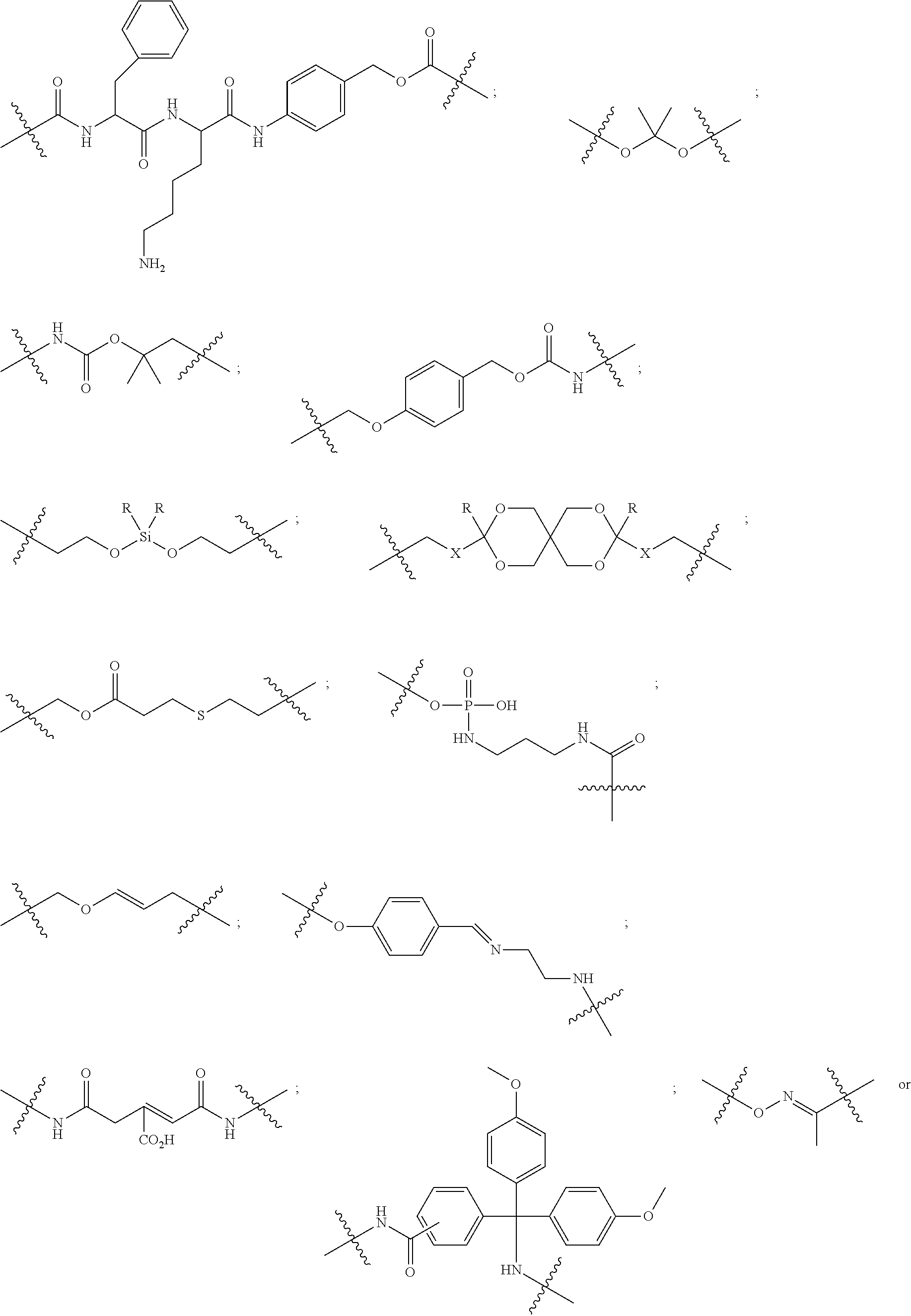

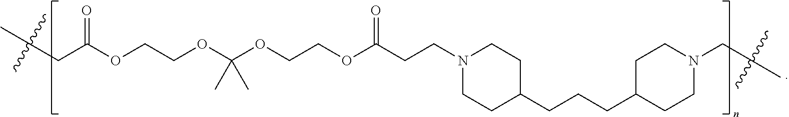

wherein:

R is H, methyl, ethyl, isopropyl, tert-butyl, or phenyl;
X is O or CH₂; and

n is an integer greater than 0.

In still other embodiments, for at least one occurrence of L^{b}, L^{b}-M has the following structure:

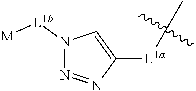

wherein L^{1a} and L^{1b} are each independently optional linkers.

In different embodiments, for at least one occurrence of L^{b}, L^{b}-M has the following structure:

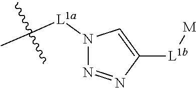

wherein L^{1a} and L^{1b} are each independently optional linkers.

In various embodiments of the foregoing, L^{1a} or L^{1b}, or both, is absent for one or more occurrences. In other embodiments, L^{1a} or L^{1b}, or both, is present for one or more occurrences.

In some embodiments L^{1a} and L^{1b}, when present, are each independently alkylene or heteroalkylene. For example, in some embodiments L^{1a} and L^{1b}, when present, independently comprise one of the following structures:

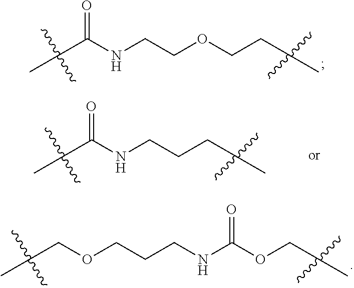 [SMILES: *CCOCCNC(*)=O ; *CCCNC(*)=O ; *COCCCNC(=O)OC*]

In some other related embodiments, L^{b} comprises one of the following structures:

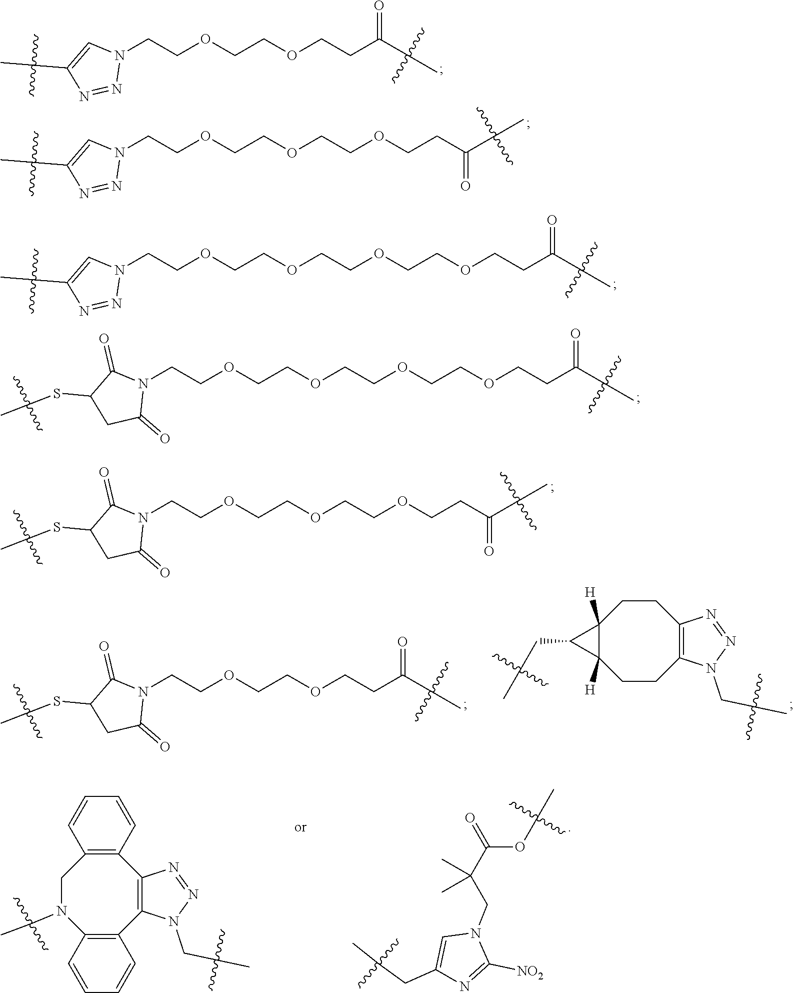

In some embodiments, at least one occurrence of L^{a} and/or L^{b} comprise one of the following structures:

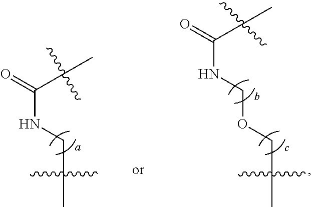

wherein

a, b, and c are each independently an integer ranging from 1-6.

In some embodiments, each occurrence of L^{a} and/or L^{b} has one of the following structures:

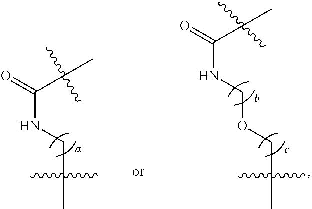

wherein:

a, b, and c are each independently an integer ranging from 1-6.

In some embodiments, at least one occurrence of L^{a} and/or L^{b} has one of the following structures:

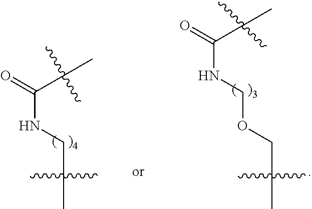

In some more specific embodiments, the compound has the following structure (IA):

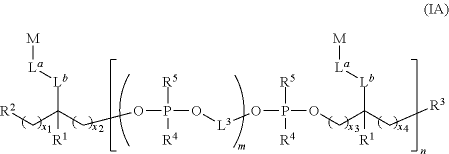

wherein:

x₁ and x₂ are each independently an integer from 0 to 10; and

x₃ and x₄ are, at each occurrence, independently an integer from 0 to 10.

In certain embodiments of the compound of structure (IA), x₁ and x₂ are each independently an integer from 0 to 3 and x₃ and x₄ are, at each occurrence, independently an integer from 0 to 3. In some other embodiments, x₁ is 1 or 0 and x₂ is 1 or 0. In more specific embodiments, x₁ is 0 and x₂ is 1. In certain embodiments, x₁ is 1 and x₂ is O.

In more embodiments, L³ is C₁-C₆ alkylene for at least one occurrence of m. In some embodiments, L³ is C₁-C₆ alkylene for each occurrence of m.

In certain more specific embodiments the compound has the following structure (IB):

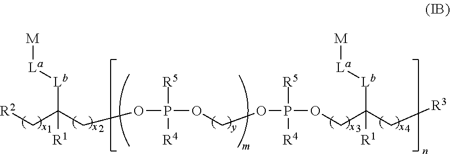

wherein:

x¹ and x² are each independently an integer from 0 to 6;

x³ and x⁴ are, at each occurrence, independently an integer from 0 to 6; and

y is an integer from 2 to 4.

In some embodiments of the compound of structure (IB), at least one occurrence of y is 2. In certain embodiments, y is 2 at each occurrence. In some embodiments, at least one occurrence of x¹, x², x³ or x⁴ is 1 and y is 2 at each occurrence.

In still other embodiments of any of the compounds of structure (I), R⁴ is, at each occurrence, independently OH, O⁻ or OR_{d}. It is understood that “OR_{d}” and “SR_{d}” are intended to refer to O⁻ and S⁻ associated with a cation. For example, the disodium salt of a phosphate group may be represented as:

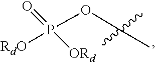

where R_{d} is sodium (Na⁺).

In other embodiments of any of the compounds of structure (I), R⁵ is, at each occurrence, oxo.

In some different embodiments of any of the foregoing compounds, le is, at each occurrence, H.

In other various embodiments, R² and R³ are each independently OH or —OP(═Rₐ)(R_{b})R_{c}. In some different embodiments, R² or R³ is OH or —OP(═Rₐ)(R_{b})R_{c}, and the other of R² or R³ is Q or a linker (e.g., an alkylene or heteroalkylene) comprising a covalent bond to Q.

In still more different embodiments of any of the foregoing compounds of structure (I), R² and R³ are each independently —OP(═Rₐ)(R_{b})R_{c}. In some of these embodiments, R_{c} is OL′. In some more specific embodiments, L′ is a targeting moiety or a linker to a targeting moiety. In related embodiments, L′ is a linker to a targeting moiety, the linker comprising an alkylene oxide or phosphodiester moiety, or combinations thereof.

In other embodiments, R² and R³ are each independently —OP(═Rₐ)(R_{b})OL′, and L′ is an alkylene or heteroalkylene linker to: Q, a targeting moiety, an analyte (e.g., analyte molecule), a solid support, a solid support residue, a nucleoside or a further compound of structure (I).

The linker L′ can be any linker suitable for attaching Q, a targeting moiety, an analyte (e.g., analyte molecule), a solid support, a solid support residue, a nucleoside or a further compound of structure (I) to the compound of structure (I). Advantageously certain embodiments include use of L′ moieties selected to increase or optimize water solubility of the compound. In certain embodiments, L′ is a heteroalkylene moiety. In some other certain embodiments, L′ comprises an alkylene oxide or phosphodiester moiety, or combinations thereof. In some other certain embodiments, L′ comprises an ethylene oxide. In some embodiments, L′ comprises a disulfide.

In certain embodiments, L′ has the following structure:

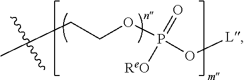

wherein:

m″ and n″ are independently an integer from 1 to 10;

R^{e} is H, an electron pair or a counter ion; L″ is the targeting moiety or a linkage to the targeting moiety.

In some embodiments, m″ is an integer from 4 to 10, for example 4, 6 or 10. In other embodiments n″ is an integer from 3 to 6, for example 3, 4, 5 or 6.

In certain embodiments, the targeting moiety is an antibody, cell surface receptor agonist, cell surface receptor antagonist or the like. In related embodiments, the antibody, cell surface receptor agonist, cell surface receptor antagonist or the like is an epidermal growth factor receptor (EGFR) inhibitor, a hepatocyte growth factor receptor (HGFR) inhibitor, an insulin-like growth factor receptor (IGFR) inhibitor, a folate, or a MET inhibitor. In certain embodiments, the antibody, cell surface receptor agonist, cell surface receptor antagonist or the like, is a tyrosine kinase inhibitor (e.g., gefitinib, erlotinib), lapatinib, Vandetanib, neratinib, osimertinib, Tovantinib (ARQ197), Crizotinib, Cabozantinib, tyrphostins (e.g., AG538, AG1024), pyrrolo(2,3-d)-pyrimidine derivatives (e.g., NVP-AEW541), monoclonal antibody (e.g., Figitumumab, Cetuximab, Panitumumab, Necitumumab, ganitumab, Cixutumumab, Dalotuzumab, Robatumumab, Onartuzumab, K1, Labetuzumab, Milatuzumab, Lorvotuzumab, Inotuzumab), BMS-777607, PF-02341066, PF-04217903, AMG-458, MK-2461, JNJ-38877605, GSK 1363089 (foretinib), XL880, XL 184, ARQ197, E7050, or INCB28060.

In some embodiments, the antibody is Abciximab, Adalimumab, Alemtuzumab, Alirocumab, Avibactam, Basiliximab, Benralizumab, Bezlotoxumab, Blinatumomab, Brodalumab, Burosumab, Canakinumab, Caplacizumab, Certolizumab pegol, Daclizumab, Denosumab, Dupilumab, Eculizumab, Emicizumab, Erenumab, Evolocumab, Fremanezumab, Galcanezumab, Golimumab, Guselkumab, Ibalizumab, Idarucizumab, Infliximab, Itolizumab, Ixekizumab, Lanadelumab, Lokivetmab, Mepolizumab, Natalizumab, Obiltoxaximab, Ocrelizumab, Omalizumab, Palivizumab, Ranibizumab, Raxibacumab, Reslizumab, Rmab, Rovelizumab, Ruplizumab, Sarilumab, Secukinumab, Tildrakizumab, Thiomab, Tocilizumab, Ustekinumab, or Vedolizumab. In certain more specific embodiments, the antibody is Abrilumab, Actoxumab, Aducanumab, Afasevikumab, Afelimomab, Anifrolumab, Anrukinzumab (IMA-638), Aselizumab, Atorolimumab, Bapineuzumab, BCD-100, Bertilimumab, Besilesomab, Biciromab, Bimagrumab, Bimekizumab, Birtamimab, Bleselumab, Blosozumab, Bococizumab, Brazikumab, Briakinumab, Brolucizumab, Carlumab, Carotuximab, Cedelizumab, Clazakizumab, Clenoliximab, Concizumab, Cosfroviximab, CR6261, Crenezumab, Crizanlizumab, Crotedumab, Depatuxizumab, mafodotin, Derlotuximab biotin, Dezamizumab, Diridavumab, Domagrozumab, Dusigitumab, Ecromeximab, Edobacomab, Efalizumab, Efungumab, Eldelumab, Elezanumab, Enokizumab, Eptinezumab, Erlizumab, Etrolizumab, Evinacumab, Exbivirumab, Fanolesomab, Faralimomab, Faricimab, Fasinumab, Felvizumab, Fezakinumab, Flanvotumab, Fletikumab, Flotetuzumab, Fontolizumab, Foravirumab, Frovocimab, Fulranumab, Gantenerumab, Gavilimomab, Gevokizumab, Gimsilumab, Gomiliximab, Gosuranemab, Ianalumab, Inclacumab, Inolimomab, Iomab-B, Keliximab, Lampalizumab, Landogrozumab, Larcaviximab, Lebrikizumab, Lenvervimab, Lerdelimumab, Letolizumab, Libivirumab, Ligelizumab, Lodelcizumab, Lulizumab pegol, Marstacimab, Mavrilimumab, Metelimumab, Mirikizumab, Motavizumab, Muromonab CD3, Nebacumab, Nemolizumab, NEOD001, Nirsevimab, Odulimomab, Olendalizumab, Olokizumab, OMS721, Opicinumab, Orticumab, Otelixizumab, Otilimab, Oxelumab, Ozanezumab, Ozoralizumab, Pagibaximab, Panobacumab, Pascolizumab, Pateclizumab, PDR001, Perakizumab, Pexelizumab, Placulumab, Plozalizumab, Ponezumab, Porgaviximab, Prasinezumab, Priliximab, PRO 140, Quilizumab, Rafivirumab, Ralpancizumab, Ranevetmab, Ravagalimab, Ravulizumab, Refanezumab, Regavirumab, Relatlimab, Rinucumab, Risankizumab, Roledumab, Romosozumab, Rontalizumab, SA237, Satralizumab, Sevirumab, SHP647, Sifalimumab, Simtuzumab, Siplizumab, Sirukumab, Solanezumab, Sonepcizumab, Spartalizumab, Stamulumab, Sulesomab, Suptavumab, Sutimlimab, Suvizumab, Suvratoxumab, Tadocizumab, Talizumab, Tamtuvetmab, Tanezumab, Tefibazumab, Telimomab aritox, Teneliximab, Teplizumab, Teprotumumab, Tezepelumab, Tibulizumab, Toralizumab, Tralokinumab, Trevogrumab, Tuvirumab, Ulocuplumab, Urtoxazumab, Varisacumab, Vepalimomab, Vesencumab, Visilizumab, Vobarilizumab, or Zolimomab aritox. In some more specific embodiments, the monoclonal antibody is trastuzumab, gemtuzumab, brentuximab, vorsetuzumab, lorvotuzumab, cantuzumab, bivatuzumabor inotuzumab, or vadastuximab.

In certain embodiments, the antibody, cell surface receptor agonist, cell surface receptor antagonist or the like, targets EGFR (e.g., EGFRvIII), HER 2, folate receptors, CD19, CD20, CD22, CD27L, CD30, CD33, CD37, CD56, CD66e, CD70, CD74, CD79b, CA6, CD138, CA 6, mesothelin, nectin 4, STEAP1, MUC16, MaPi2b, GCC, Trop-2, AGS-5, ENPP3, carbonic anhydrase IX, GPNMB, PDMA,

In certain other embodiments, the antibody, cell surface receptor agonist, cell surface receptor antagonist or the like, targets 1-40-β-amyloid; activated F9, F10; ACVR2B; amyloids; Ang-2; angiopoietin 3; anthrax toxin, protective antigen; AOC3 (VAP-1); Bacillus anthracis anthrax; BAFF; beta-amyloid; C1s; C5; calcitonin; calcitonin gene-related peptide alpha; Canis lupus familiaris IL31; CCL11(eotaxin-1); CCR2; CCR5; CD11; CD18; CD125; CD147 (basigin); CD15; CD154 (CD40L); CD19; CD2; CD20; CD23 (IgE receptor); CD25 (a chain of IL-2 receptor); CD28; CD3; CD4; CD40; CD41 (integrin alpha-IIb); CD45; CD5; CD52; CD6; CEA-related antigen; CFD; CGRP; Clostridium difficile; clumping factor A; complement C5a; CSF2; CXCR4 (CD184); cytomegalovirus; dabigatran; ebolavirus glycoprotein; EGFR; endoglin; endotoxin; Escherichia coli; F protein of respiratory syncytial virus; FGF 23; fibrin II, beta chain; GCGR; GD3ganglioside; GDF-8; GMCSF; growth differentiation factor 8; hemagglutinin; hepatitis B surface antigen; histone complex; HIV-1; HNGF; Hsp90; human beta-amyloid; human TNF; IgE; IGF-1receptor (CD221); IGHE; Influenza A hemagglutinin; integrin receptors and subunits; interferon receptors; interleukin receptors (various); ITGB2 (CD18); kallikrein; LAG3; LFA-1 (CD11a); LINGO-1; lipoteichoic acid; LOXL2; L-selectin (CD62L); LTA; MASP-2; MCP-1; mucosal addressin cell adhesion molecule; myelin-associated glycoprotein; myostatin; NACP; NCA-90 (granulocyte antigen); neural apoptosis-regulated proteinase 1; NGF; NOGO-A; NRP1; OX-40; oxLDL; PCSK9; PD-1; PDCD1, CD279; platelet-derived growth factor receptor beta; Pseudomonas aeruginosa; rabies virus glycoprotein; RANKL; respiratory syncytial virus; RGMA; RHD; Rhesus factor; RSVFR; sclerostin; selectin P; SOST; sphingosine-1-phosphate; Staphylococcus aureus alpha toxin; tau protein; TFPI; TGF beta 1; TGF beta 2; TNF-α; TRAP; TSLP; TYRP1(glycoprotein 75); VEGF-A; VWF; Zaire ebolavirus glycoprotein.

In some specific embodiments, the targeting moiety is an antibody or antibody fragment. In certain more specific embodiments, the antibody or antibody fragment is a monoclonal antibody (mAb), antigen binding fragment (Fab/Fab′), single-domain antibody (sdAb), bispecific antibody (BsAb), bispecific t-cell engager (BiTE), single-chain variable fragment (ScFv), dual-affinity re-targeting antibodies (DARTs), heavy chain variable domain (V_{H}), minibodies, diabodies, or Abdurins™ (derived from IgG).

In some other embodiments, the targeting moiety is a protein. For example, in some embodiments, the targeting moiety is albumin, interferon, centyrin, or chemotaxis receptor ligand.

In some other embodiments, L″ is an alkylene or heteroalkylene moiety. In some other certain embodiments, L″ comprises an alkylene oxide, phosphodiester moiety, sulfhydryl, disulfide or maleimide moiety or combinations thereof.

In other more specific embodiments of any of the foregoing compounds of structure (I), R² or R³ has one of the following structures:

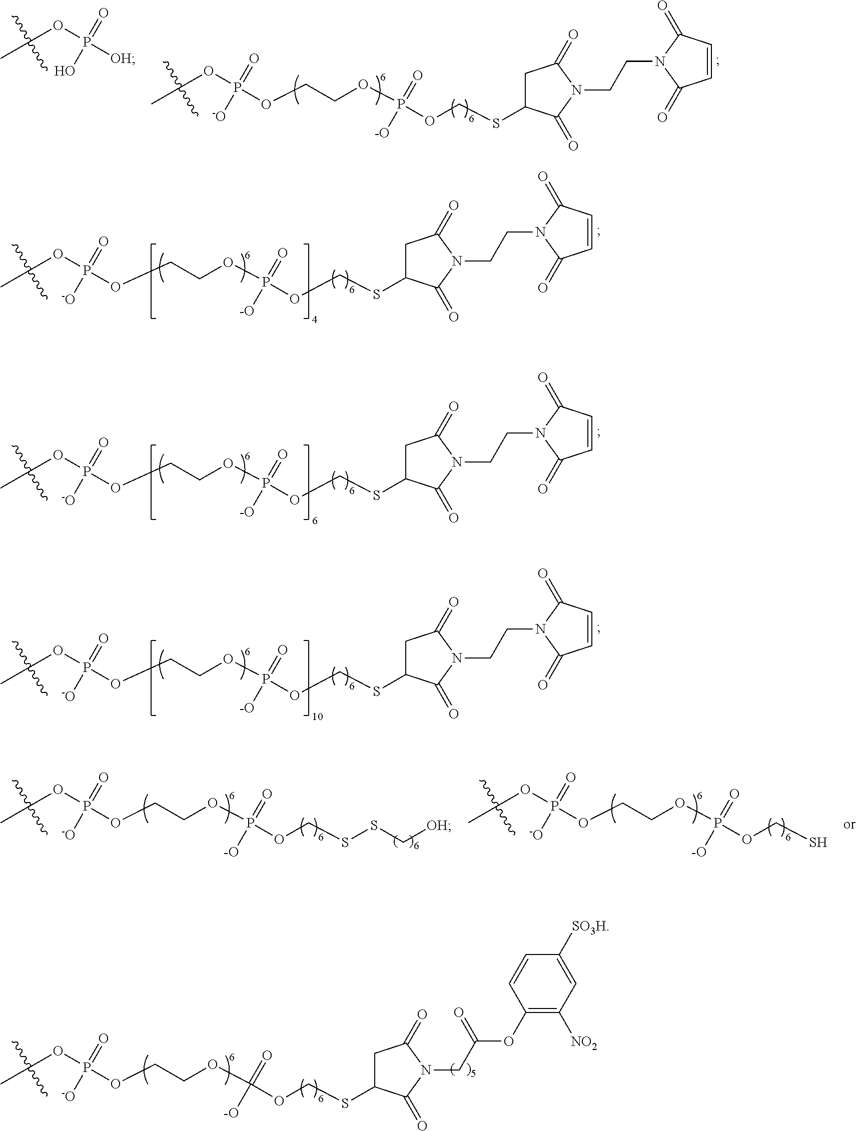

Certain embodiments of compounds of structure (I) can be prepared according to solid-phase synthetic methods analogous to those known in the art for preparation of oligonucleotides. Accordingly, in some embodiments, L′ is a linkage to a solid support, a solid support residue, or a nucleoside. Solid supports (e.g., polymeric and non-polymeric) comprising an activated deoxythymidine (dT) group are readily available, and in some embodiments can be employed as starting material for preparation of compounds of structure (I). Accordingly, in some embodiments R² or R³ has the following structure:

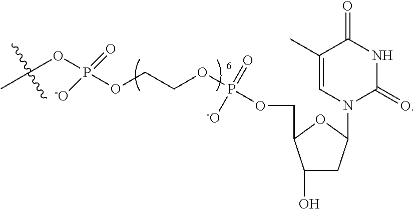

One of skill in the art will understand that the dT group depicted above is included for ease of synthesis and economic efficiencies only, and is not required. Other solid supports can be used and would result in a different nucleoside or solid support residue being present on L′, or the nucleoside or solid support residue can be removed or modified post synthesis.

In some embodiments, R² or R³ comprises one of the following structures:

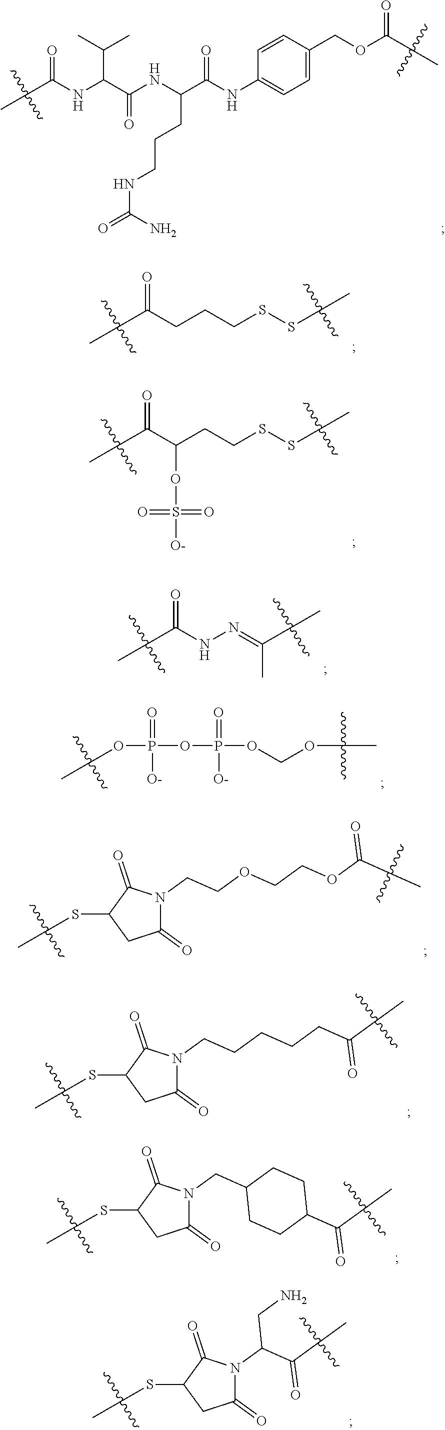

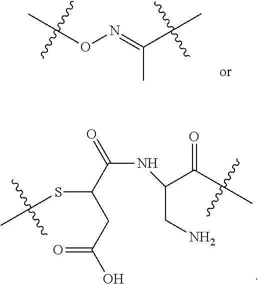

In some embodiments, R² or R³ comprises one of the following structures:

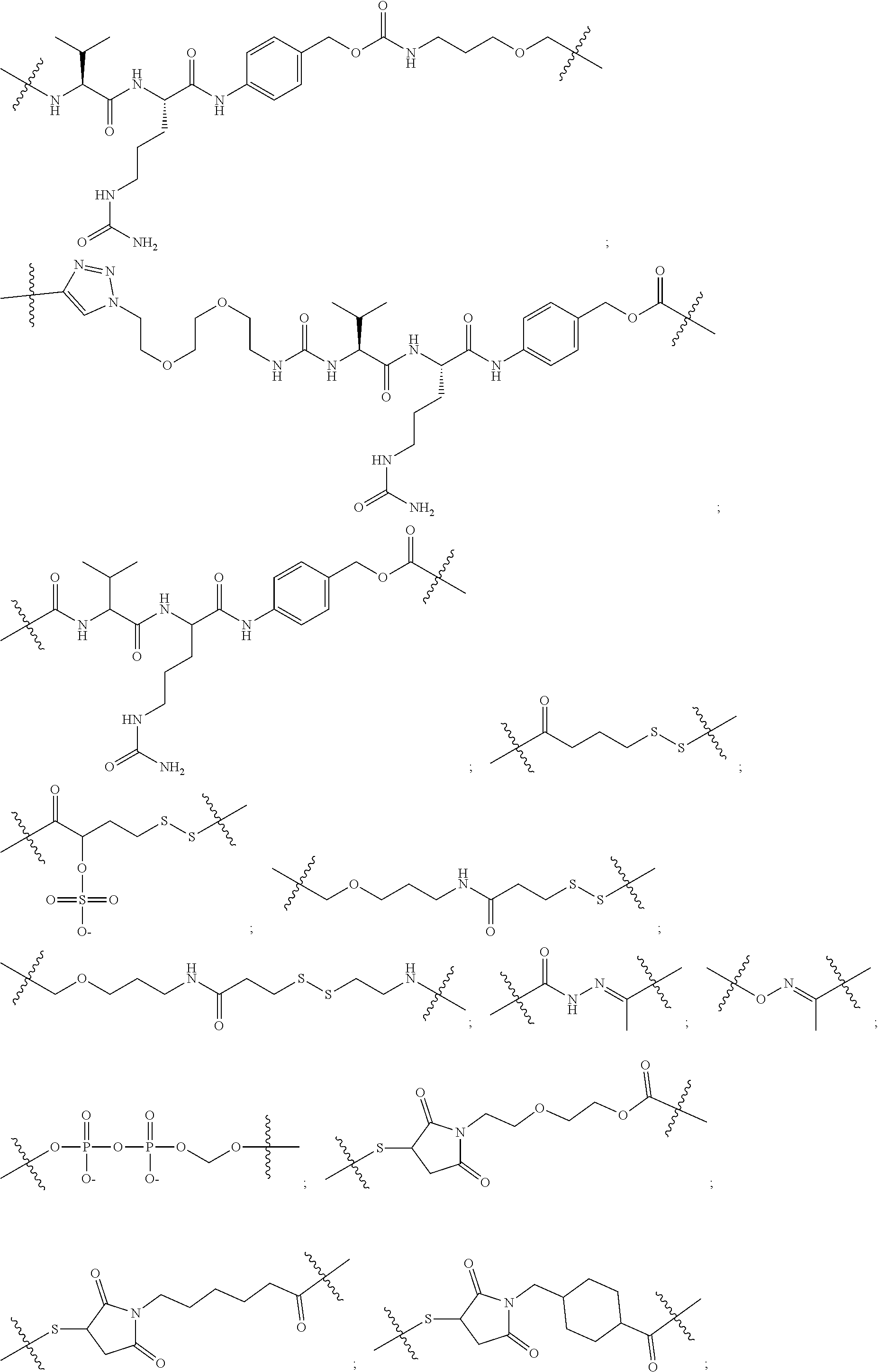

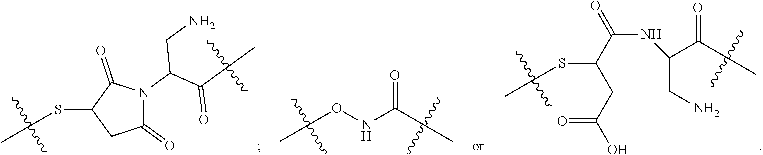 [SMILES: *SC1CC(=O)N(C(CN)C(*)=O)C1=O ; *ONC(*)=O ; *SC(CC(=O)O)C(=O)NC(CN)C(*)=O]

In some embodiments, R³ comprises one of the following structures:

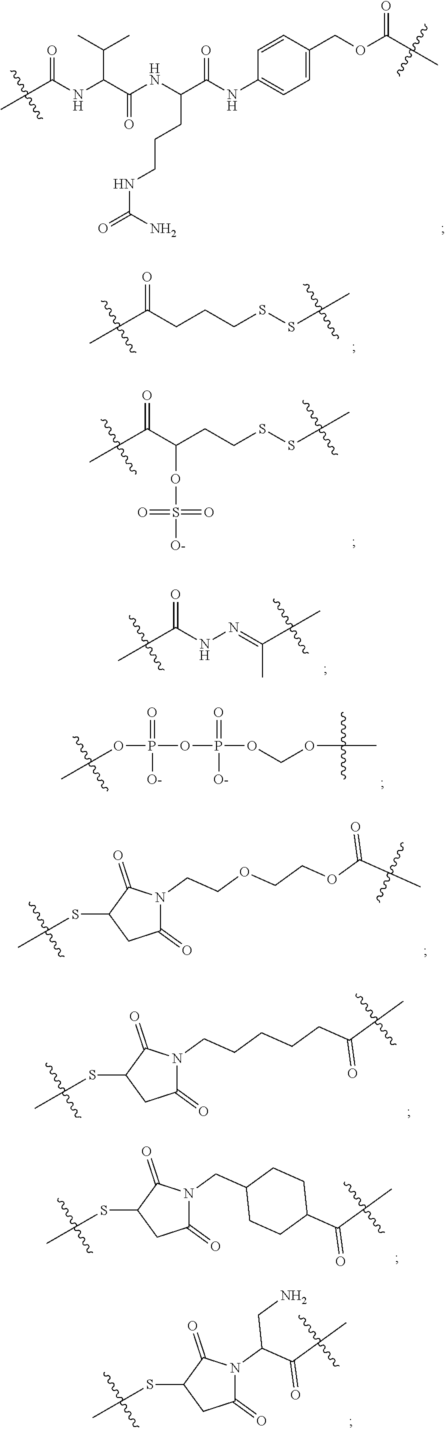

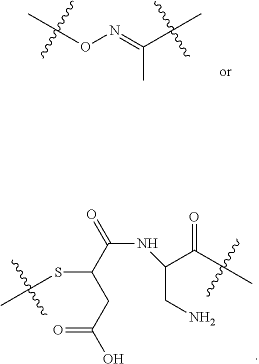

In some embodiments, R³ comprises one of the following structures:

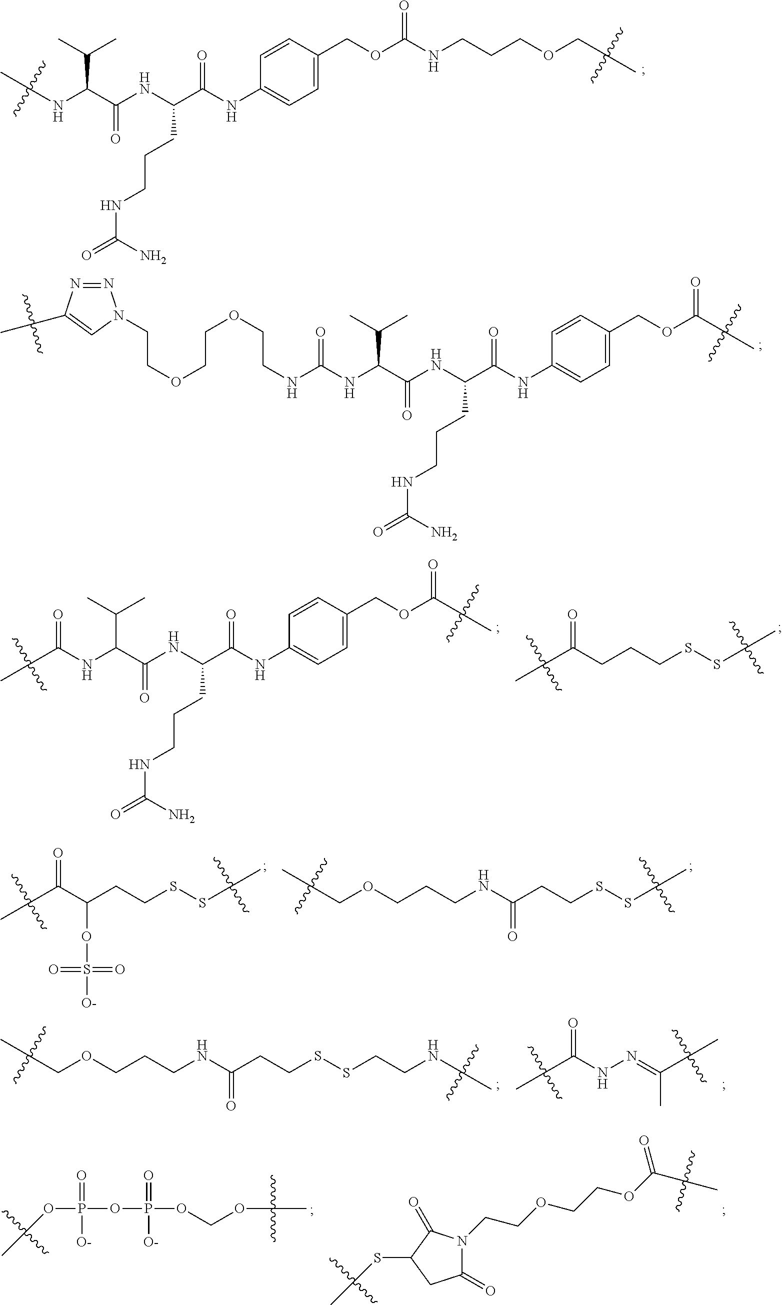

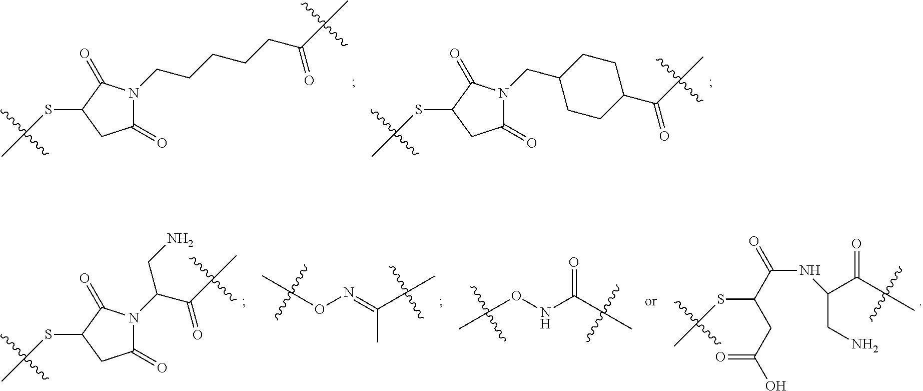

The values of m and n are variables that can be selected based on the desired solubility, permeation effect, or therapeutic application. In other embodiments, n is, at each occurrence, independently an integer from 1 to 5, for example 1, 2, 3, 4 or 5. The solubility, permeation or retention can also be tuned by selection of different values of n. In certain embodiments, n is an integer from 1 to 100. In other embodiments, n is an integer from 1 to 10. In some embodiments n is 1. In some embodiments n is 2. In some embodiments n is 3. In some embodiments n is 4. In some embodiments n is 5. In some embodiments n is 6. In some embodiments n is 7. In some embodiments n is 8. In some embodiments n is 9. In some embodiments n is 10. In certain embodiments, n is an integer from 1 to 10.

In some embodiments, m is an integer from 1 to 10. In more specific embodiments, at least one occurrence of m is an integer from 1 to 5. In certain embodiments, each occurrence of m is an integer from 1 to 15. In some embodiments, each occurrence of m is an integer from 1 to 10. In more embodiments, each occurrence of m is an integer from 1 to 5.

In still other embodiments, Q is, at each occurrence, independently a moiety comprising a reactive group capable of forming a covalent bond with an analyte molecule or a solid support. In other embodiments, Q is, at each occurrence, independently a moiety comprising a reactive group capable of forming a covalent bond with a complementary reactive group Q′. For example, in some embodiments, Q′ is present on a further compound of structure (I) (e.g., in the R² or R³ position), and Q and Q′ comprise complementary reactive groups such that reaction of the compound of structure (I) and the further compound of structure (I) results in covalently bound dimer of the compound of structure (I). Multimer compounds of structure (I) can also be prepared in an analogous manner and are included within the scope of embodiments of the disclosure.

The type of Q group and connectivity of the Q group to the remainder of the compound of structure (I) is not limited, provided that Q comprises a moiety having appropriate reactivity for forming the desired bond.

In certain embodiments, Q is a moiety which is not susceptible to hydrolysis under aqueous conditions, but is sufficiently reactive to form a bond with a corresponding group on an analyte molecule or solid support (e.g., an amine, azide or alkyne).

Certain embodiments of compounds of structure (I) comprise Q groups commonly employed in the field of bio-conjugation. For example in some embodiments, Q comprises a nucleophilic reactive group, an electrophilic reactive group or a cycloaddition reactive group. In some more specific embodiments, Q comprises a sulfhydryl, disulfide, activated ester, isothiocyanate, azide, alkyne, alkene, diene, dienophile, acid halide, sulfonyl halide, phosphine, α-haloamide, biotin, amino or maleimide functional group. In some embodiments, the activated ester is an N-succinimide ester, imidoester or polyflourophenyl ester. In other embodiments, the alkyne is an alkyl azide or acyl azide.

The Q groups can be conveniently provided in protected form to increase storage stability or other desired properties, and then the protecting group removed at the appropriate time for coupling with, for example, a targeting moiety or analyte. Accordingly, Q groups include “protected forms” of a reactive group, including any of the reactive groups described above and in the Table 1 below. A “protected form” of Q refers to a moiety having lower reactivity under predetermined reaction conditions relative to Q, but which can be converted to Q under conditions, which preferably do not degrade or react with other portions of the compound of structure (I). One of skill in the art can derive appropriate protected forms of Q based on the particular Q and desired end use and storage conditions. For example, when Q is SH, a protected form of Q includes a disulfide, which can be reduce to reveal the SH moiety using commonly known techniques and reagents.

Exemplary Q moieties are provided in Table I below.

TABLE 1
Exemplary Q Moieties

| Structure | Class |
|---|---|
| 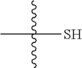 [SMILES: *S] | Sulfhydryl |
| 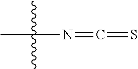 [SMILES: *N=C=S] | Isothiocyanate |
| 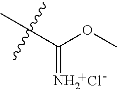 | Imidoester |
| 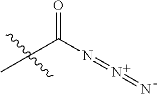 | Acyl Azide |
| 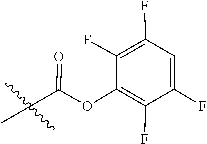 [SMILES: *C(=O)Oc1c(F)c(F)cc(F)c1F] | Activated Ester |
| 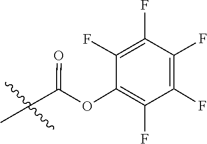 [SMILES: *C(=O)Oc1c(F)c(F)c(F)c(F)c1F] | Activated Ester |
| 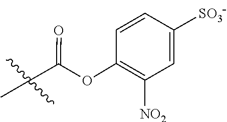 | Activated Ester |
| 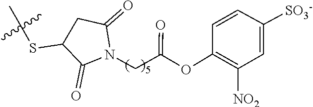 | Activated Ester |
| 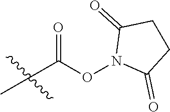 [SMILES: *C(=O)ON1C(=O)CCC1=O] | Activated Ester |
| 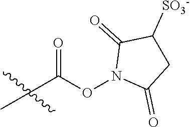 | Activated Ester |
| 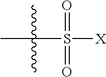 | Sulfonyl halide |
| X = halo | |
| 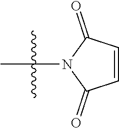 [SMILES: *N1C(=O)C=CC1=O] | Maleimide |
| 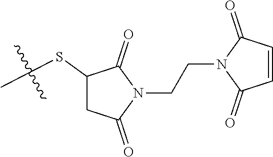 [SMILES: *SC1CC(=O)N(CCN2C(=O)C=CC2=O)C1=O] | Maleimide |
| 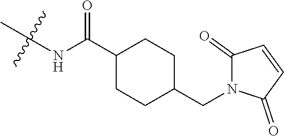 [SMILES: *NC(=O)C1CCC(CN2C(=O)C=CC2=O)CC1] | Maleimide |
| 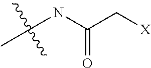 | α-haloamide |
| X = halo | |
| 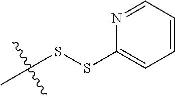 [SMILES: *SSc1ccccn1] | Disulfide |
| 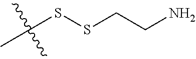 [SMILES: *SSCCN] | Disulfide |
| 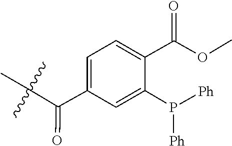 [SMILES: *C(=O)c1ccc(C(=O)OC)c(P(c2ccccc2)c2ccccc2)c1] | Phosphine |
| 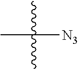 [SMILES: *N=[N+]=[N-]] | Azide |
| 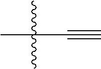 [SMILES: *C#C] | Alkyne |
| 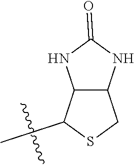 [SMILES: *C1SCC2NC(=O)NC21] | Biotin |
| 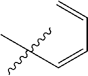 [SMILES: */C=C\C=C] | Diene |
| 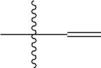 [SMILES: *C=C] | Alkene/dienophile |
| 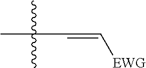 | Alkene/dienophile |
| EWG = eletron withdrawing group | |
| —NH₂ | Amino |

It should be noted that in some embodiments, wherein Q is SH, the SH moiety will tend to form disulfide bonds with another sulfhydryl group, for example on another compound of structure (I). Accordingly, some embodiments include compounds of structure (I), which are in the form of disulfide dimers, the disulfide bond being derived from SH Q groups.

Also included within the scope of certain embodiments are compounds of structure (I), wherein one, or both, of R² and R³ comprises a linkage to a further compound of structure (I). For example, wherein one or both of R² and R³ are —OP(═Rₐ)(R_{b})R_{c}, and R_{c} is OL′, and L′ is a linker comprising a covalent bond to a further compound of structure (I). Such compounds can be prepared by preparing a first compound of structure (I) having for example about 10 “M” moieties (i.e., n=9) and having an appropriate “Q” for reaction with a complementary Q′ group on a second compound of structure (I). In this manner, compounds of structure (I), having any number of “M” moieties, for example 100 or more, can be prepared without the need for sequentially coupling each monomer. Exemplary embodiments of such compounds of structure (I) have the following structure (I′)

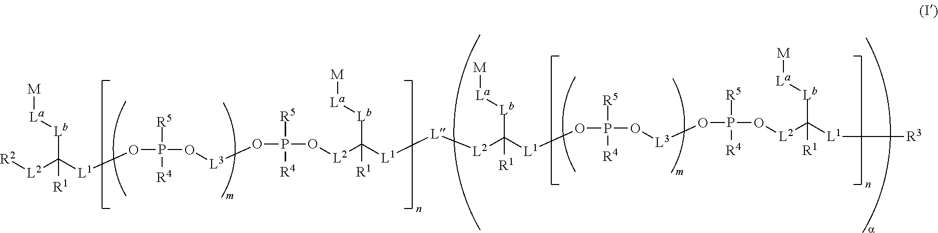

wherein:

each occurrence of R¹, R², R³, R⁴, R⁵, L^{a}, L^{b}, L¹, L², L³, M, m, and n are independently as defined for a compound of structure (I);

L″ is a linker comprising a functional group resulting from reaction of a Q moiety (e.g., as in Table 1) with a corresponding Q′ moiety; and

α is an integer greater than 1, for example from 1 to 100, or 1 to 10.

Other compounds of structure (I′) are derivable by those of ordinary skill in the art, for example by dimerizing or polymerizing compounds of structure (I) provided herein.

In other embodiments, the Q moiety is conveniently masked (e.g., protected) as a disulfide moiety, which can later be reduced to provide an activated Q moiety for binding to a desired targeting moiety. For example, the Q moiety may be masked as a disulfide having the following structure:

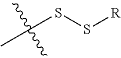

wherein R is an optionally substituted alkyl group. For example, in some embodiments, Q is provided as a disulfide moiety having the following structure:

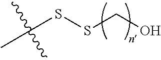

wherein n′ is an integer from 1 to 10, for example 6.

In some other embodiments, one of R² or R³ is OH or —OP(═Rₐ)(R_{b})R_{c}, and the other of R² or R³ is a linker comprising a covalent bond to a targeting moiety or a linker comprising a covalent bond to a solid support. For example, in some embodiments the targeting moiety is an antibody, cell surface receptor agonist, cell surface receptor antagonist or the like. In still different embodiments, the solid support is a polymeric bead or non-polymeric bead. The targeting moiety may be directed to any number of strategic targets. For example, the biological target may be a cell surface receptor such as a tumor cell antigen. Tumor cell antigens include tumor-specific antigens and tumor-associated antigens, for example EGFR, HER 2, folate receptors, CD20, CD33, oncofetal antigens (e.g., alphafetoprotein, carcinoembryonic antigen, immature laminin receptor, TAG-72), CA-125, MUC-1, epithelial tumor antigen, tyrosinase, melanoma-associated antigen (MAGE), and abnormal products of RAS or p53. Tumor cell antigens may also include antigens characterized as oncofetal, oncoviral (e.g., HPV E6, E7), overexpressed/accumulated (e.g., BING-4, calcium activated chloride channel 2, 9D7, Ep-CAM, EphA3, HER2, telomerase, mesothelin, SAP-1, survivin), cancer-tetis (e.g., BAGE family, CAGE family, GAGE family, MAGE family, SAGE family, XAGE family), lineage-restricted, mutated, post-translationally altered, idiotypic, CT9 or CT10 (e.g., NY-ESO-1/LAGE-1, PRAME).

In some embodiments, M is at each occurrence, independently an NSAID, a kinase inhibitor, an anthracycline, and EGFR inhibitor or an alkylating agent. In some embodiments, the biologically active moiety is an anti-cancer drug. In certain specific embodiments, M is at each occurrence, independently an anti-cancer drug, and the targeting moiety is an antibody specific for a tumor cell antigen.

Anti-cancer drug, as used herein, includes derivatives. That is, and anti-cancer drug that has been modified or derivatized such that the drug can be conjugated or attached to another molecule (e.g., to include Q moieties). For example, maytansine is a cancer drug and maytansinoids are cancer drug derivatives.

In certain embodiments, the anti-cancer drug is an epidermal growth factor receptor (EGFR) inhibitor, phosphatidylinositol kinase (PI3K) inhibitor, insulin-like growth factor receptor (IGF1R) inhibitor, Janus kinase (JAK) inhibitor, a Met kinase inhibitor, a SRC family kinase inhibitor, a mitogen-activated protein kinase (MEK) inhibitor, an extracellular-signal-regulated kinase (ERK) inhibitor, a topoisomerase inhibitors (such as irinotecan, or such as etoposide, or such asdoxorubicin), taxanes (such as anti-microtubule agents including paclitaxel and docetaxel), anti-metabolite agents (such as 5-FU or such as gemcitabine), alkylating agents (such as cisplatin or such as cyclophosphamide), or a taxane.

Anti-cancer drugs that can be modified and incorporated into embodiments of compounds of the present disclosure include, for example, auristatin F; auristatin E; maytansine; calicheamicin; paclitaxel; doxorubicin; cryptophycin; erlotinib; CC-1065; carzelesin; SJG-136; DSB-120; afatinib; Iressa; methotrexate; DNA methylation agents (e.g., procarbazine, temozolomide, dacarbazine, N-methylo-N-nitrourea, N-methyl-N′-nitro-N-nitroguanine, and the like).

Other non-limiting examples of anti-cancer drugs include Gleevec® (Imatinib Mesylate), Velcade® (bortezomib), Casodex (bicalutamide), Iressa® (gefitinib), and Adriamycin, alkylating agents such as thiotepa and cyclosphosphamide) (CYTOXAN®; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex®, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins or capecitabine. Also included as suitable cancer drugs are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomeRASe inhibitor RFS 2000; difluoromethylornithine (DMFO), Duocarmycin A, and Pemetrexed (Alimta).

Where desired, embodiments of the compounds or composition of the present disclosure can be used in combination with commonly prescribed anti-cancer drugs such as Herceptin®, Avastin, Erbitux, Rituxan, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BMW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

M is selected based on the desired therapeutic and/or optical properties, for example based on treating a specific disease or condition (e.g., cancer) or producing a particular color and/or fluorescence emission wavelength. In some embodiments, M is the same at each occurrence; however, it is important to note that each occurrence of M need not be an identical M, and certain embodiments include compounds wherein M is not the same at each occurrence. For example, in some embodiments, each M is not the same and the different M moieties are selected to have different therapeutic properties (e.g., cytotoxic and anti-inflammatory). In some embodiments, each M is not the same and the different M moieties are selected to have the same or similar therapeutic properties (e.g., cytotoxicity).

Accordingly, in some embodiments, at least one occurrence of M is an antineoplastic agent, an enediyne antitumor antibiotic, a maytansinoid, a topoisomerase inhibitor, a kinase inhibitor, an anthracycline, and EGFR inhibitor or an alkylating agent. In certain embodiments, at least one occurrence of M is an antineoplastic agent, an enediyne antitumor antibiotic, a maytansinoid, a topoisomerase inhibitor, or an alkylating agent. In some specific embodiments, M is, at each occurrence, independently an antineoplastic agent, an enediyne antitumor antibiotic, a maytansinoid, a topoisomerase inhibitor, a kinase inhibitor, an anthracycline, and EGFR inhibitor or an alkylating agent. In certain specific embodiments, M is, at each occurrence, independently an antineoplastic agent, an enediyne antitumor antibiotic, a maytansinoid, a topoisomerase inhibitor, or an alkylating agent.

In some embodiments, at least one occurrence of M is selected from the group consisting of auristatin F, monomethyl auristatin F, monomethyl auristatin E, paciltaxol, SN-38, calicheamicin, anthramycin, abbeymycin, chicamycin, DC-81, mazethramycin, neothramycin A, neothramycin B, porothramycin prothracarcin, sibanomicin, sibiromycin, tomamycin, mertansine, emtansine, irinotecan, camptothecin, topotecan, silatecan, cositecan, Exatecan, Lurtotecan, gimatecan, Belotecan, and Rubitecan. In certain embodiments, at least one occurrence of M has one of the following structures:

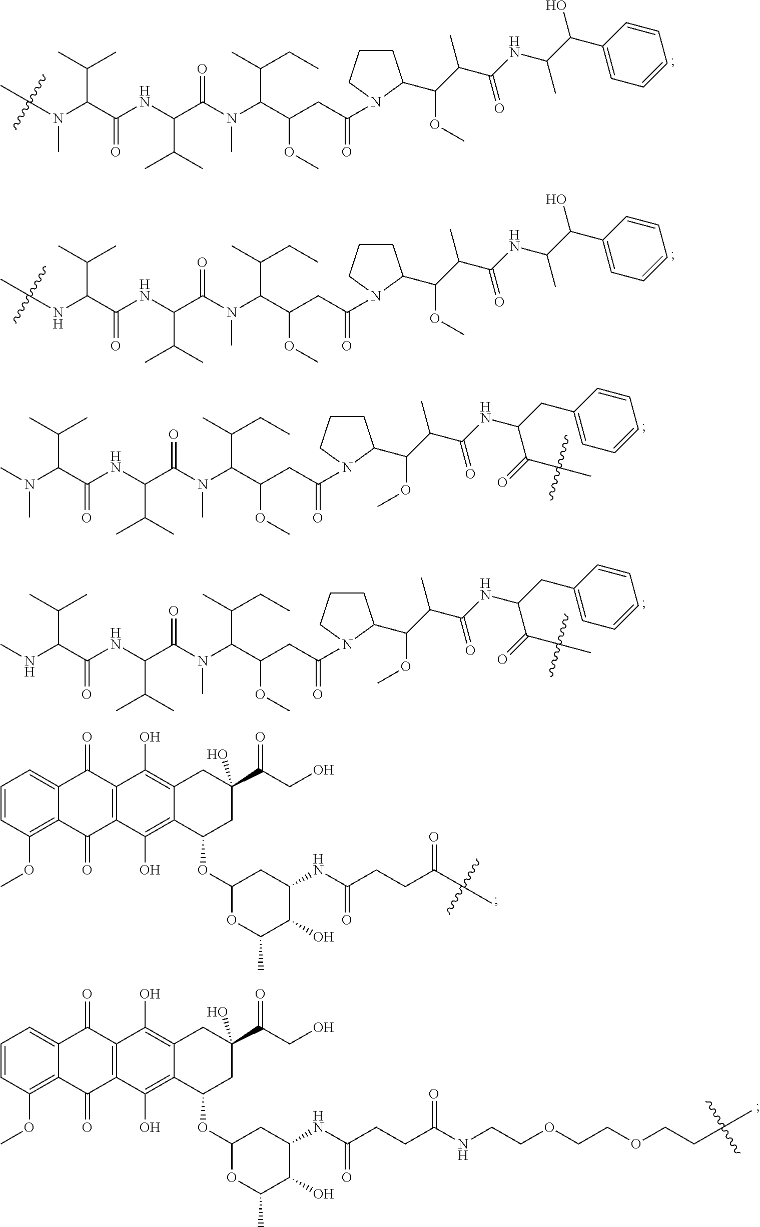

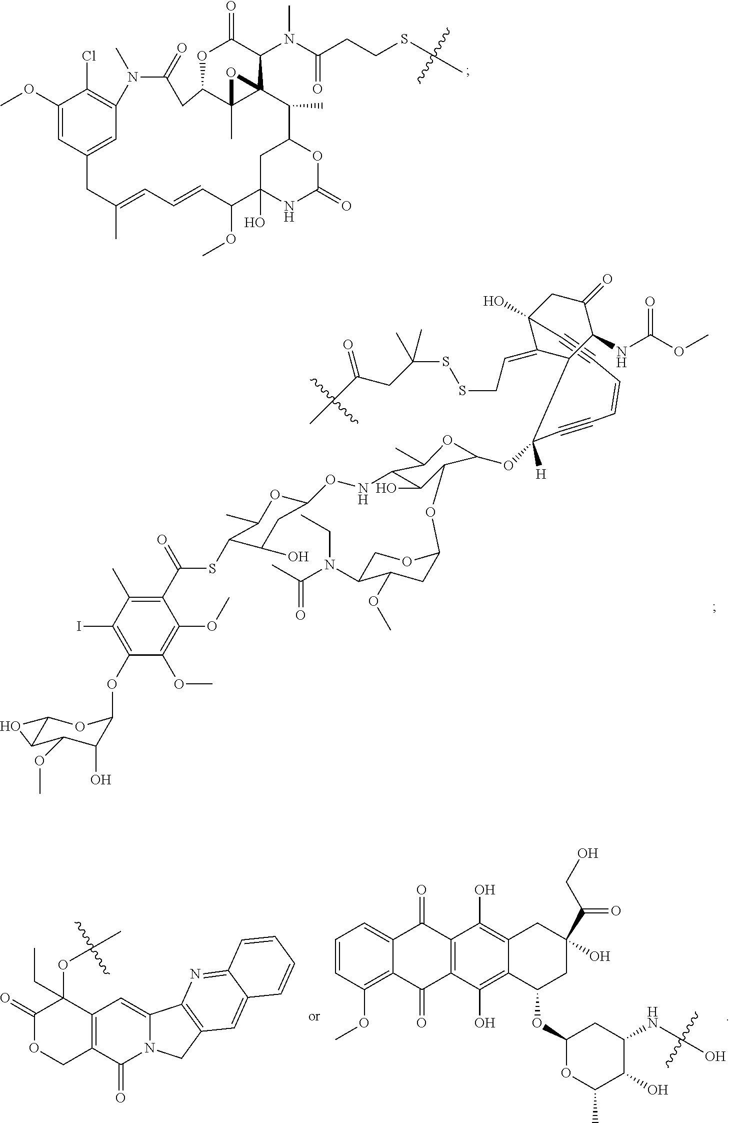

In some embodiments, each occurrence of M has one of the following structures:

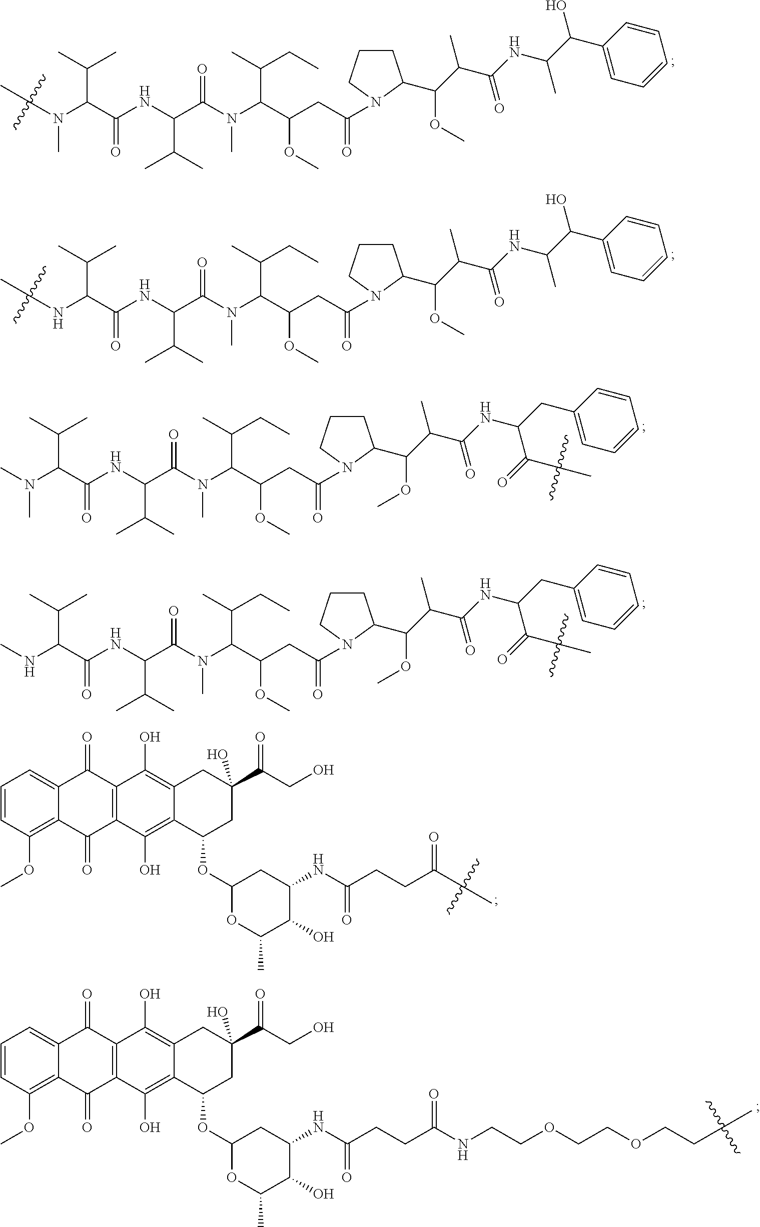

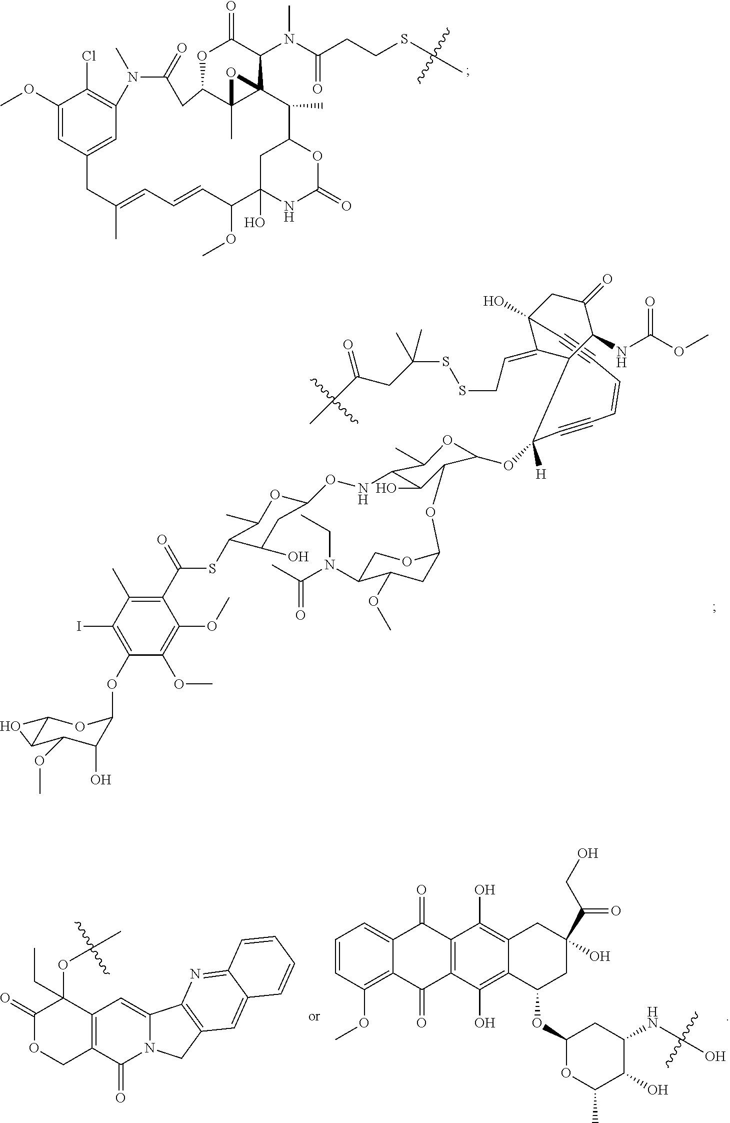

In certain embodiments, each M is independently selected from the following:

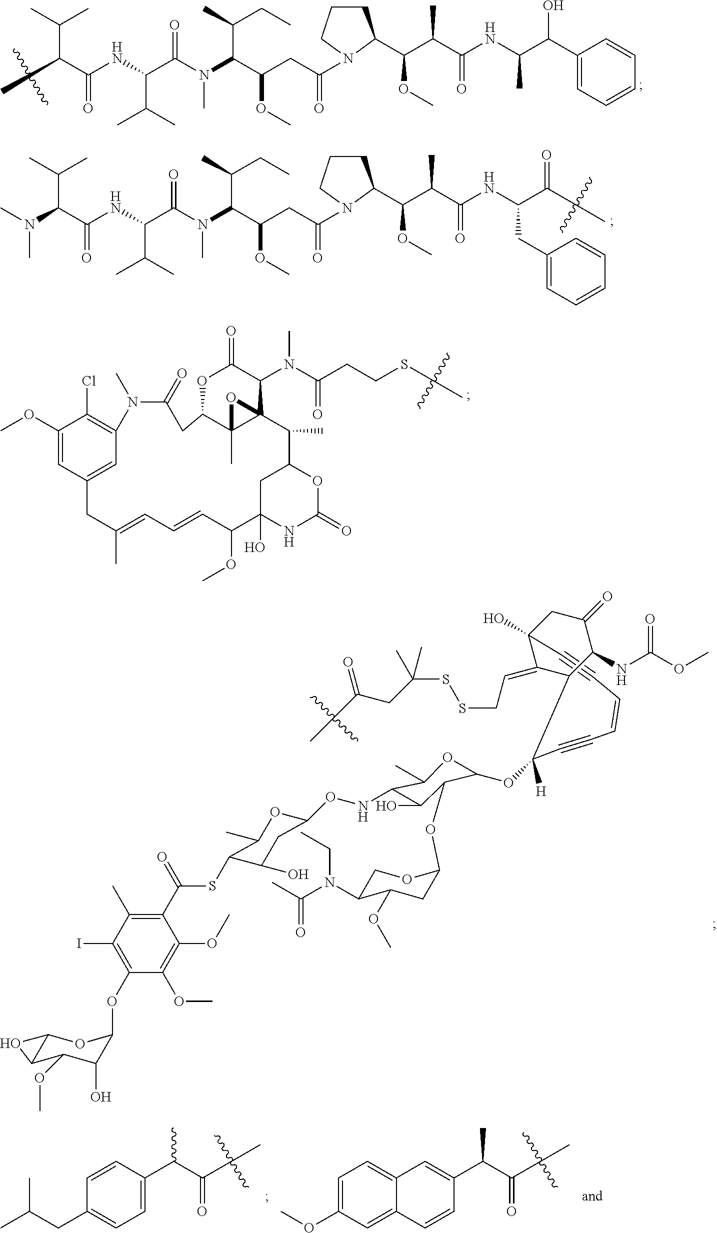

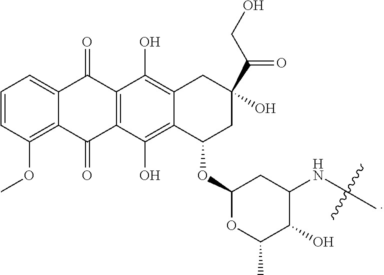 [SMILES: *NC1C[C@H](O[C@H]2C[C@](O)(C(=O)CO)Cc3c(O)c4c(c(O)c32)C(=O)c2c(OC)cccc2C4=O)O[C@@H](C)[C@H]1O]

Although depicted having specific points of attachment (i.e., [symbol] with the remainder of the molecule for ease of illustration, the M moiety may be attached via any available point (e.g., at a nitrogen, oxygen, carboxy, carbonyl, etc.). One of skill in the art can determine an appropriate attachment point.

In some embodiments, at least one occurrence of M is an antineoplastic agent (e.g., auristatin F, monomethyl auristatin F, monomethyl auristatin E, paciltaxol, SN-38), an enediyne antitumor antibiotic (e.g., calicheamicin or more specifically calicheamicin γ1), an alkylating agent (e.g., a PBD or pyrollo benzo diazepines), a maytansinoids (e.g., mertansine, emtansine) a topoisomerase inhibitor (e.g., SN38, irinotecan, camptothecin, topotecan, silatecan, cositecan, Exatecan, Lurtotecan, gimatecan, Belotecan, Rubitecan).

In some embodiments, each occurrence of M is an antineoplastic agent (e.g., auristatin F, monomethyl auristatin F, monomethyl auristatin E, paciltaxol, SN-38), an enediyne antitumor antibiotic (e.g., calicheamicin or more specifically calicheamicin yl), an alkylating agent (e.g., a PBD or pyrollo benzo diazepines), a maytansinoids (e.g., mertansine, emtansine) a topoisomerase inhibitor (e.g., SN38, irinotecan, camptothecin, topotecan, silatecan, cositecan, Exatecan, Lurtotecan, gimatecan, Belotecan, Rubitecan).

In certain embodiments, L^{a}-M has the following structure:

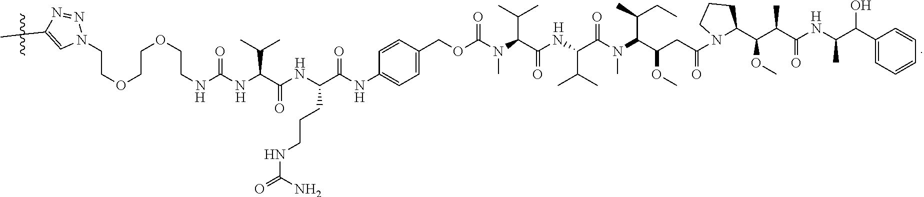

In certain embodiments, L^{b}-M has one of the following structures:

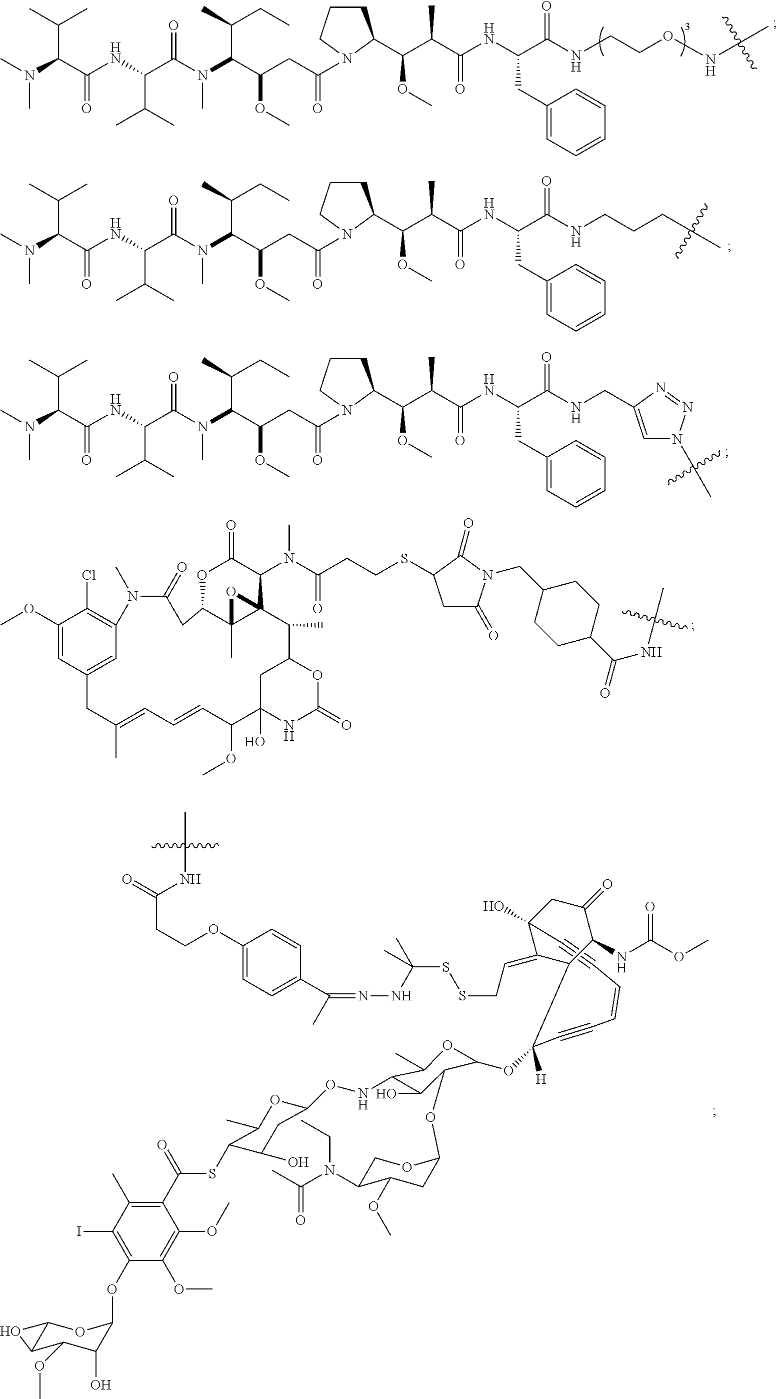

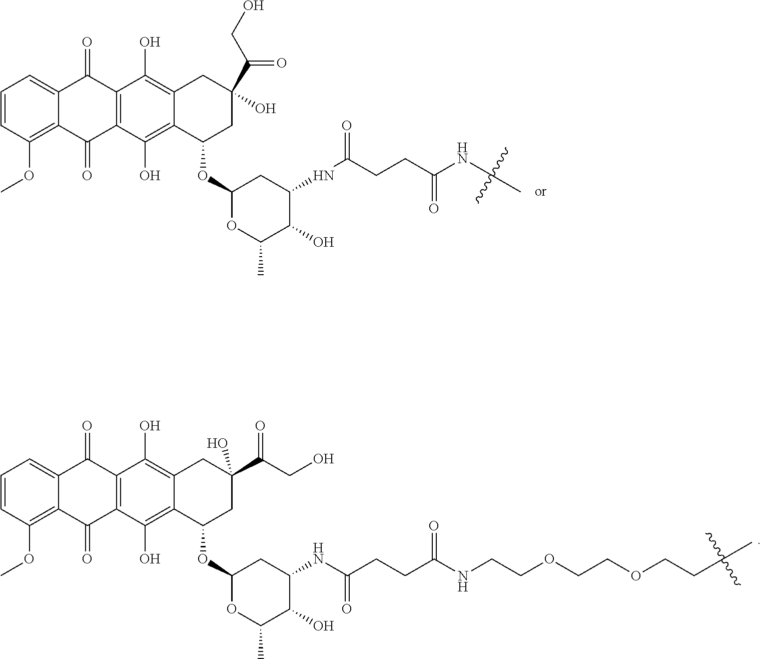 [SMILES: *NC(=O)CCC(=O)N[C@H]1C[C@H](O[C@H]2C[C@](O)(C(=O)CO)Cc3c(O)c4c(c(O)c32)C(=O)c2c(OC)cccc2C4=O)O[C@@H](C)[C@H]1O ; *CCOCCOCCNC(=O)CCC(=O)N[C@H]1C[C@H](O[C@H]2C[C@](O)(C(=O)CO)Cc3c(O)c4c(c(O)c32)C(=O)c2c(OC)cccc2C4=O)O[C@@H](C)[C@H]1O]

In still more embodiments of any of the foregoing, M is the same. In other embodiments, each M is different. In still more embodiments, one or more M is the same and one or more M is different.

In some embodiments selected occurrences of M are not the same and the different M moieties are selected to have absorbance and/or emissions for use in fluorescence resonance energy transfer (FRET) methods. For example, in such embodiments the different M moieties are selected such that absorbance of radiation at one wavelength causes emission of radiation at a different wavelength by a FRET mechanism. Exemplary M moieties can be appropriately selected by one of ordinary skill in the art based on the desired end use. Exemplary M moieties for FRET methods include fluorescein and 5-TAMRA (5-carboxytetramethylrhodamine, succinimidyl ester) dyes.

M may be attached to the remainder of the molecule from any position (i.e., atom) on M. One of skill in the art will recognize means for attaching M to the remainder of molecule. Exemplary methods include the “click” reactions described herein.

In some embodiments, M is a fluorescent or colored moiety. Any fluorescent and/or colored moiety may be used, for examples those known in the art and typically employed in colorimetric, UV, and/or fluorescent assays may be used. Examples of M moieties which are useful in various embodiments of the disclosure include, but are not limited to: Xanthene derivatives (e.g., fluorescein, rhodamine, Oregon green, eosin or Texas red); Cyanine derivatives (e.g., cyanine, indocarbocyanine, oxacarbocyanine, thiacarbocyanine or merocyanine); Squaraine derivatives and ring-substituted squaraines, including Seta, SeTau, and Square dyes; Naphthalene derivatives (e.g., dansyl and prodan derivatives); Coumarin derivatives; oxadiazole derivatives (e.g., pyridyloxazole, nitrobenzoxadiazole or benzoxadiazole); Anthracene derivatives (e.g., anthraquinones, including DRAQ5, DRAQ7 and CyTRAK Orange); Pyrene derivatives such as cascade blue; Oxazine derivatives (e.g., Nile red, Nile blue, cresyl violet, oxazine 170); Acridine derivatives (e.g., proflavin, acridine orange, acridine yellow); Arylmethine derivatives: auramine, crystal violet, malachite green; and Tetrapyrrole derivatives (e.g., porphin, phthalocyanine or bilirubin). Other exemplary M moieties include: Cyanine dyes, xanthate dyes (e.g., Hex, Vic, Nedd, Joe or Tet); Yakima yellow; Redmond red; tamra; texas red and alexa Fluor® dyes.

In still other embodiments of any of the foregoing, M comprises three or more aryl or heteroaryl rings, or combinations thereof, for example four or more aryl or heteroaryl rings, or combinations thereof, or even five or more aryl or heteroaryl rings, or combinations thereof. In some embodiments, M comprises six aryl or heteroaryl rings, or combinations thereof. In further embodiments, the rings are fused. For example in some embodiments, M comprises three or more fused rings, four or more fused rings, five or more fused rings, or even six or more fused rings.

In some embodiments, M is cyclic. For example, in some embodiments M is carbocyclic. In other embodiments, M is heterocyclic. In still other embodiments of the foregoing, M, at each occurrence, independently comprises an aryl moiety. In some of these embodiments, the aryl moiety is multicyclic. In other more specific examples, the aryl moiety is a fused-multicyclic aryl moiety, for example which may comprise at least 3, at least 4, or even more than 4 aryl rings.

In other embodiments of any of the foregoing compounds, M comprises at least one heteroatom. For example, in some embodiments, the heteroatom is nitrogen, oxygen or sulfur.

In still more embodiments of any of the foregoing, M comprises at least one substituent. For example, in some embodiments the substituent is a fluoro, chloro, bromo, iodo, amino, alkylamino, arylamino, hydroxy, sulfhydryl, alkoxy, aryloxy, phenyl, aryl, methyl, ethyl, propyl, butyl, isopropyl, t-butyl, carboxy, sulfonate, amide, or formyl group.

In some even more specific embodiments of the foregoing, M is a dimethylaminostilbene, quinacridone, fluorophenyl-dimethyl-BODIPY, his-fluorophenyl-BODIPY, acridine, terrylene, sexiphenyl, porphyrin, benzopyrene, (fluorophenyl-dimethyl-difluorobora-diaza-indacene)phenyl, (bis-fluorophenyl-difluorobora-diaza-indacene)phenyl, quaterphenyl, bi-benzothiazole, ter-benzothiazole, bi-naphthyl, bi-anthracyl, squaraine, squarylium, 9, 10-ethynylanthracene or ter-naphthyl moiety. In other embodiments, M is p-terphenyl, perylene, azobenzene, phenazine, phenanthroline, acridine, thioxanthrene, chrysene, rubrene, coronene, cyanine, perylene imide, or perylene amide or a derivative thereof. In still more embodiments, M is a coumarin dye, resorufin dye, dipyrrometheneboron difluoride dye, ruthenium bipyridyl dye, energy transfer dye, thiazole orange dye, polymethine or N-aryl-1,8-naphthalimide dye.

In some embodiments, M is pyrene, perylene, perylene monoimide or 6-FAM or a derivative thereof. In some other embodiments, M has one of the following structures:

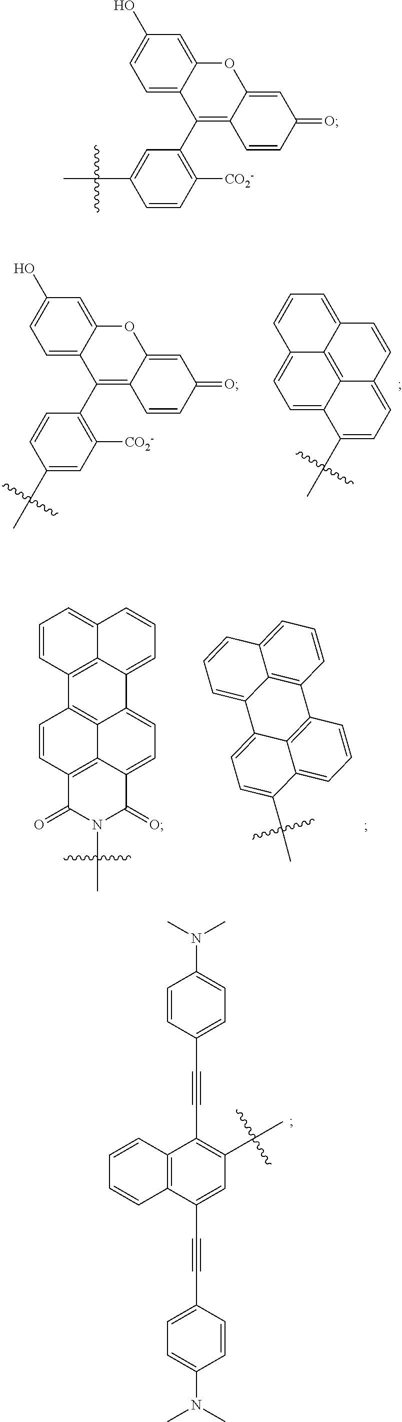

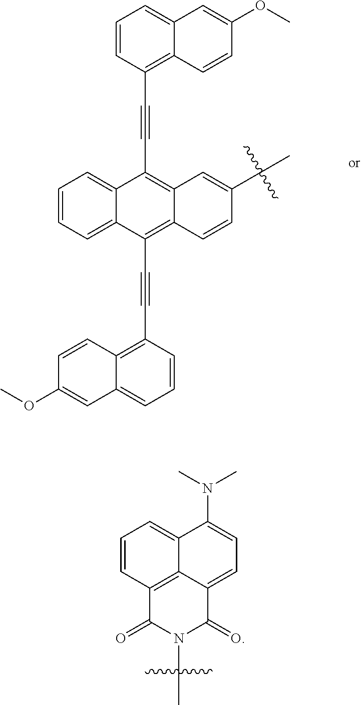

Although M moieties comprising carboxylic acid groups are depicted in the anionic form (CO₂⁻) above, one of skill in the art will understand that this will vary depending on pH, and the protonated form (CO₂H) is included in various embodiments.

In some specific embodiments, the compound is a compound selected from Table 2. The compounds in Table 2 were prepared according to the procedures set forth in the Examples and their identity can be confirmed by mass spectrometry.

TABLE 2
Exemplary Compounds of Structure I

| Cpd. | Structure |
|---|---|
| I-1 | 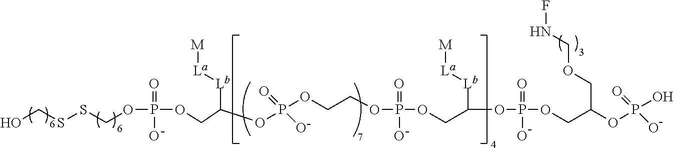 |

As used in Table 2 and throughout the application M has the definitions provided for compounds of structure (I) unless otherwise indicated. In some embodiments, M is F, F′, F″, N′, I′, D′, D″, or AF. F, F′ and F″ refer to a fluorescein moiety having the following structures, respectively:

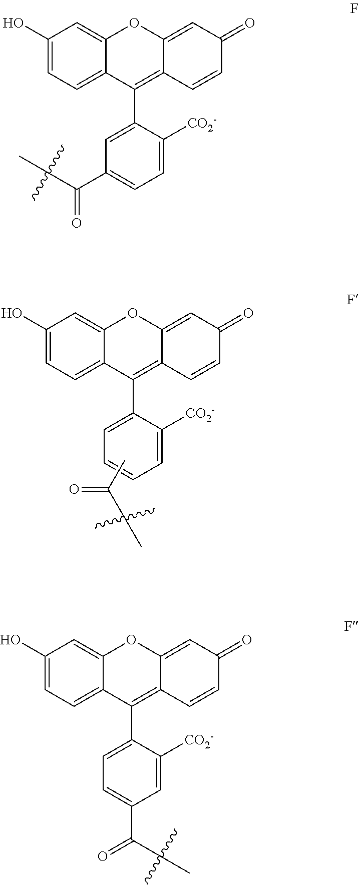

“N′” refers to the following structure:

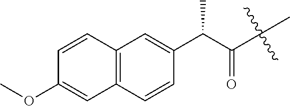 [SMILES: *C(=O)[C@@H](C)c1ccc2cc(OC)ccc2c1]

“I′” refers to the following structure:

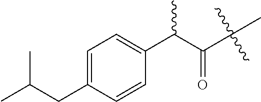 [SMILES: *C(=O)C(C)c1ccc(CC(C)C)cc1]

“D′” refers to the following structure:

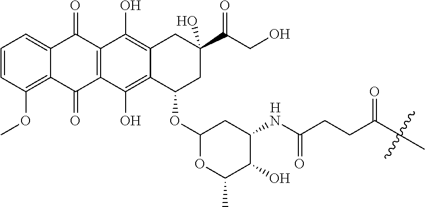

“D″” refers to the following structure:

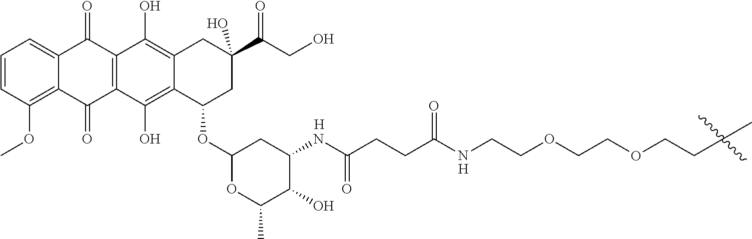

“dT” refers to the following structure:

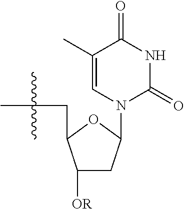

wherein:

R is H or a direct bond.

“AF” refers to the following structure:

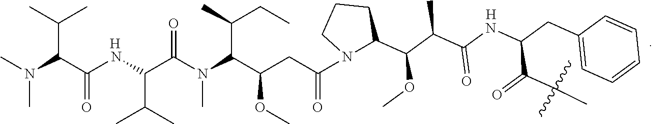

Accordingly, in some embodiments, at least one occurrence of M has one of the following structures:

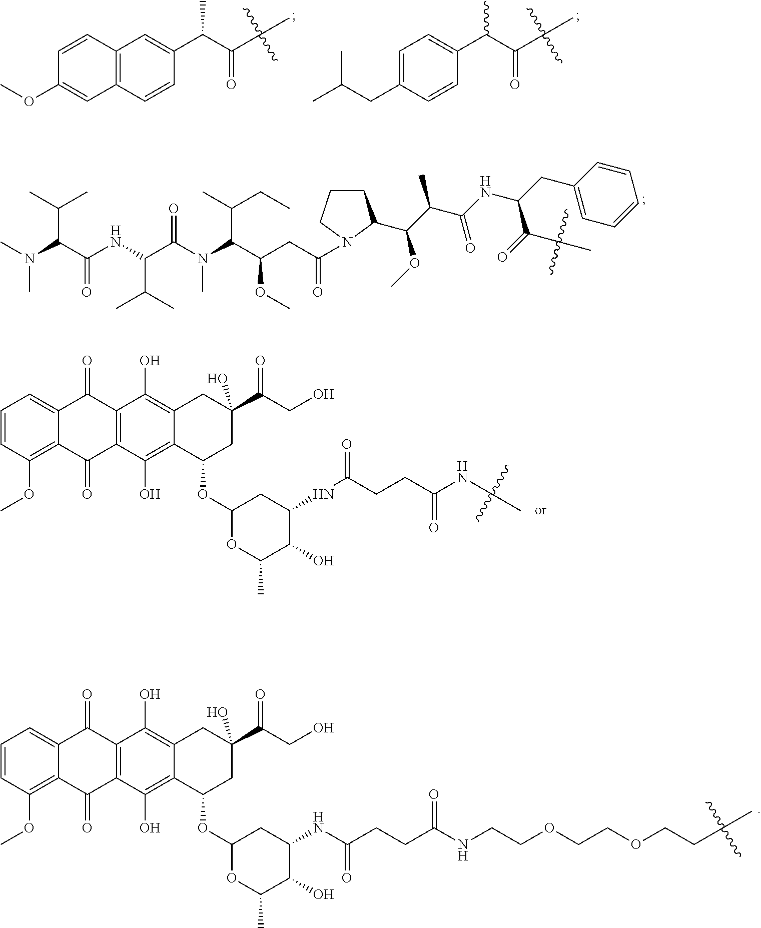

In some more specific embodiments, each occurrence of M has the following structure:

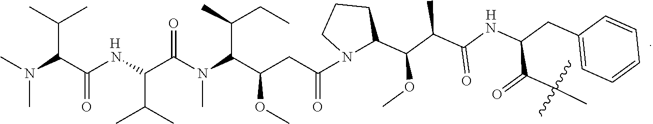

In certain embodiments, treating includes reducing or alleviating pain or inflammation. In certain embodiments, treating includes pain control or pain management. In some specific embodiments, at least one occurrence of M has one of the following structures:

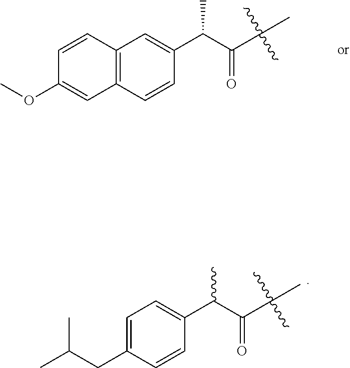 [SMILES: *C(=O)[C@@H](C)c1ccc2cc(OC)ccc2c1 ; *C(=O)C(C)c1ccc(CC(C)C)cc1]

In some more specific embodiments, each occurrence of M has one of the following structure:

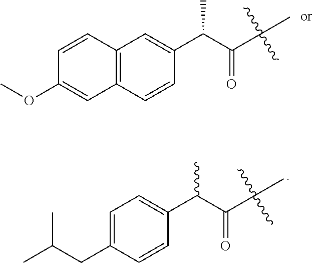 [SMILES: *C(=O)[C@@H](C)c1ccc2cc(OC)ccc2c1 ; *C(=O)C(C)c1ccc(CC(C)C)cc1]

Some embodiments include any of the foregoing compounds, including the specific compounds provided in Table 2, conjugated to a targeting moiety, such as an antibody. In some embodiments, one compound of structure (I) is conjugated to an antibody. In some embodiments, 1-2 compounds of structure (I) are conjugated to an antibody. In some embodiments, 2 compounds of structure (I) are conjugated to an antibody. In some embodiments, 3 compounds of structure (I) are conjugated to an antibody. In some embodiments, 4 compounds of structure (I) are conjugated to an antibody. In some embodiments, 5 compounds of structure (I) are conjugated to an antibody. In some embodiments, no more than 5 compounds of structure (I) are conjugated to an antibody.

In various embodiments, reactive polymers can be used to prepare compounds of structure (I). In certain embodiments, these reactive polymers are synthetic intermediates that comprise a moiety useful for reacting with a complementary moiety to form a covalent bond between M and the reactive polymer via any number of synthetic methodologies (e.g., the “click” reactions described above), thereby forming a compound of structure (I). Accordingly, in various embodiments a compound of structure (I) is formed using a reactive polymer having the following structure (II):

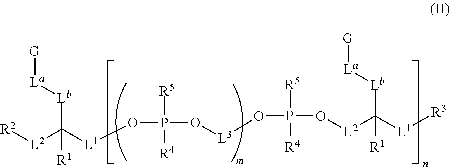

or a stereoisomer, salt or tautomer thereof, wherein:

G is, at each occurrence, independently a moiety comprising a reactive group, or protected analogue thereof, capable of forming a covalent bond with a complementary reactive groups;

L^{a} is, at each occurrence, independently an optional physiologically cleavable linker and L^{b} is, at each occurrence, independently an optional physiologically non-cleavable linker, provided that at least one occurrence of L^{a} and L^{b} together comprise more than 4 carbons;

L¹ and L² are, at each occurrence, independently an optional alkylene, alkenylene, alkynylene, heteroalkylene, heteroalkenylene, heteroalkynylene or heteroatomic linker;

L³ is, at each occurrence, independently an alkylene, alkenylene, alkynylene, heteroalkylene, heteroalkenylene or heteroalkynylene linker;

R¹ is, at each occurrence, independently H, alkyl or alkoxy;

R² and R³ are each independently H, OH, SH, alkyl, alkoxy, alkylether, heteroalkyl, —OP(═Rₐ)(R_{b})R_{c}, Q, or a protected form thereof, or L′;

R⁴ is, at each occurrence, independently O⁻, S⁻, OZ, SZ or N(R⁶)₂, where Z is a cation and each R⁶ is independently H or alkyl;

R⁵ is, at each occurrence, independently oxo, thioxo or absent;

Rₐ is O or S;

R_{b} is OH, SH, O⁻, S⁻, ORₐ or SR_{d};

R_{c} is OH, SH, O⁻, S⁻, ORₐ, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;

Rₐ is a counter ion;

Q is, at each occurrence, independently a moiety comprising a reactive group, or protected form thereof, capable of forming a covalent bond with a complementary reactive group Q′ on a targeting moiety;

L′ is, at each occurrence, independently a linker comprising a covalent bond to Q, a targeting moiety, a linker comprising a covalent bond to a targeting moiety, a linker comprising a covalent bond to a solid support, a linker comprising a covalent bond to a solid support residue, a linker comprising a covalent bond to a nucleoside or a linker comprising a covalent bond to a further compound of structure (I);

m is, at each occurrence, independently an integer of zero or greater; and

n is an integer of one or greater.

In some embodiments, one occurrence of G comprises a fluorescent dye moiety.

In some embodiments, the reactive polymer is selected from Table 3, below.

TABLE 3
Exemplary Reactive Polymers of structure (II)

| Cpd. | Structure |
|---|---|
| II-1 | 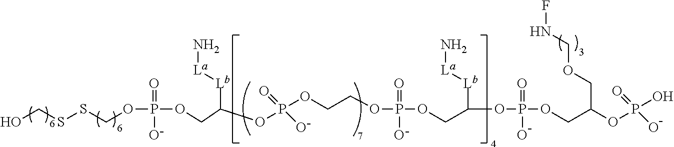 |

Certain embodiments are directed to a therapeutically effective fluorescent compound provided at least one occurrence of M is not a fluorescent dye and at least one occurrence of M is a fluorescent dye. Therapeutically effective fluorescent compounds include compounds comprising at least one biologically active moiety or fragment thereof or a prodrug of a biologically active moiety, or fragment thereof, which emit a fluorescent signal upon excitation with light, such as ultraviolet light.

Embodiments of the presently disclosed compounds are “tunable,” meaning that by proper selection of the variables in any of the foregoing compounds, one of skill in the art can arrive at a compound having a desired and/or predetermined molar fluorescence (molar brightness). The “tunability” of certain embodiments of the compounds allows the user to easily arrive at compounds having the desired fluorescence and/or color for use in a particular assay. Although all variables may have an effect on the molar fluorescence of certain embodiments of the compounds disclosed herein, proper selection of M, L³, m and n is believed to play an important role in the molar fluorescence of embodiments of the disclosed compounds. Accordingly, in one embodiment is provided a method for obtaining a compound having a desired molar fluorescence, the method comprising selecting an M moiety having a known fluorescence, preparing a compound of structure (I) comprising the M moiety, and selecting the appropriate variables for L³, m and n to arrive at the desired molar fluorescence.

For ease of illustration, various compounds comprising phosphorous moieties (e.g., phosphate and the like) are depicted in the anionic state (e.g., —OPO(OH)O⁻, —OPO₃²⁻). One of skill in the art will readily understand that the charge is dependent on pH and the uncharged (e.g., protonated or salt, such as sodium or other cation) forms are also included in the scope of embodiments of the disclosure.

Compositions comprising any of the foregoing compounds and one or more targeting moiety (e.g., antibody, cell surface receptor agonist, cell surface receptor antagonist or the like) are provided in various other embodiments. In some embodiments, use of such compositions in methods for treating a disease the method comprising administering to a subject in need thereof a therapeutically effective amount of a compound of structure (I) or a composition comprising a compound of structure (I) wherein each M is independently a biologically active moiety effective for treating the disease are also provided.

### Pharmaceutical Compositions

One embodiment provides a composition comprising the compound according any one of the embodiments disclosed herein and a pharmaceutically acceptable carrier.

Another embodiment provides a composition comprising a plurality of conjugates, the conjugates comprising an antibody covalently bound to two or more biologically active moieties via a single linkage (e.g., a compound of structure (I)), wherein the plurality of conjugates has at least 90% structural homogeneity. In more specific embodiments, the plurality of conjugates has at least 95% structural homogeneity. In related embodiments, the plurality of conjugates has greater than 99% structural homogeneity. In certain embodiments, the single linkage is a linkage to a polymer backbone, the polymer backbone comprising the two or more biologically active moieties covalently bound thereto. In some of the foregoing embodiments, the conjugate or conjugates comprise a compound of structure (I).

In certain embodiments, each conjugate independently is a compound of structure (I), wherein one of R² and R³ is —OP(═Rₐ)(R_{b}) OL′ or L′, and L′ is the antibody or a linker comprising a covalent bond to the antibody.

Other embodiments are directed to pharmaceutical compositions. The pharmaceutical composition comprises any one (or more) of the compounds of structure (I) and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound of structure (I) and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound of structure (I) is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound of structure (I) is provided in the form of a rapid release formulation, in the form of an extended release formulation, or in the form of an intermediate release formulation. In yet other embodiments, the compound of structure (I) is administered topically.

The compounds of structure (I) are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that are used in some embodiments. An exemplary dosage is 10 to 30 mg per day. The exact dosage will depend upon the route of administration, the form in which the compound is administered, the subject to be treated, the body weight of the subject to be treated, and the preference and experience of the attending physician.

In some embodiments, a compound of structure (I) is administered in a single dose. Typically, such administration will be by injection, e.g., intravenous injection, in order to introduce the agent quickly. However, other routes are used as appropriate. A single dose of a compound of structure (I) may also be used for treatment of an acute condition.

In some embodiments, a compound of structure (I) is administered in multiple doses. In some embodiments, dosing is about once, twice, three times, four times, five times, six times, or more than six times per day. In other embodiments, dosing is about once a month, once every two weeks, once a week, or once every other day. In another embodiment a compound of structure (I) and another agent are administered together about once per day to about 6 times per day. In another embodiment the administration of a compound of structure (I) and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of structure (I) may continue as long as necessary. In some embodiments, a compound of structure (I) is administered for more than 1, 2, 3, 4, 5, 6, 7, 14, or 28 days. In some embodiments, a compound of structure (I) is administered for less than 28, 14, 7, 6, 5, 4, 3, 2, or 1 day. In some embodiments, a compound of structure (I) is administered chronically on an ongoing basis, e.g., for the treatment of chronic effects.

In some embodiments, the compounds of structure (I) are administered in dosages. It is known in the art that due to intersubject variability in compound pharmacokinetics, individualization of dosing regimen is necessary for optimal therapy. Dosing for a compound of the disclosure may be found by routine experimentation in light of the instant disclosure.

In some embodiments, the compounds of structure (I) are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound of structure (I) and a pharmaceutically acceptable diluent(s), excipient(s), or carrier(s). In certain embodiments, the compounds described are administered as pharmaceutical compositions in which compounds of structure (I) are mixed with other active ingredients, as in combination therapy. Encompassed herein are all combinations of actives set forth in the combination therapies section below and throughout this disclosure. In specific embodiments, the pharmaceutical compositions include one or more compounds of structure (I).

A pharmaceutical composition, as used herein, refers to a mixture of a compound of structure (I) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds of structure (I) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds of structure (I) are used singly or in combination with one or more therapeutic agents as components of mixtures.

In one embodiment, one or more compounds of structure (I) is formulated in an aqueous solution. In specific embodiments, the aqueous solution is selected from, by way of example only, a physiologically compatible buffer, such as Hank's solution, Ringer's solution, or physiological saline buffer. In other embodiments, one or more compound of structure (I) is/are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler.

Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels. In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds (e.g., compounds of structure (I)) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In still other embodiments, the compounds of structure (I) are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compounds of structure (I) are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the compounds of structure (I) is accomplished by means of iontophoretic patches and the like. In certain embodiments, transdermal patches provide controlled delivery of the compounds of structure (I). In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, the compounds of structure (I) are formulated for administration by inhalation. Various forms suitable for administration by inhalation include, but are not limited to, aerosols, mists or powders. Pharmaceutical compositions of any of compound of structure (I) are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator is formulated containing a powder mix of the compound and a suitable powder base such as lactose or starch.

In still other embodiments, the compounds of structure (I) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with melted cocoa butter.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising a compound of structure (I) are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound of structure (I), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least one compound of structure (I) illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of a compound of structure (I). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of one or more compounds provided in the pharmaceutical compositions is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more compounds is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more compounds is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more compounds ranges from 0.0001 to 10 g, 0.0005 to 9 g, 0.001 to 8 g, 0.005 to 7 g, 0.01 to 6 g, 0.05 to 5 g, 0.1 to 4 g, 0.5 to 4 g, or 1 to 3 g.

### Methods of Treatment

Compounds of the present disclosure are useful for treating disease. The compounds disclosed herein offer a targeted approach to drug delivery strategies.

Additionally, compounds of structure (I) offer a distinct advantage over previously known compounds due to their ability to include virtually any therapeutic moiety. Biologically active moieties (e.g., therapeutic agents) can be reversibly or irreversibly attached and delivered to a target.

Accordingly, in some embodiments, the compounds are useful in various methods of treating a disease or condition. One embodiment provides a method of treating a disease, the method comprising administering to a subject in need thereof a therapeutically effective amount of a compound of structure (I) or a composition comprising a compound of structure (I), wherein at least one M is a biologically active moiety effective for treating the disease. In more specific embodiments, each M is a biologically active moiety effective for treating the disease.

In some embodiments, the disease biologically active moiety degrades proteins. In some more specific embodiments, the protein is an amyloid or tau protein. In certain embodiments, the disease is amyloidosis or Alzheimer's disease. In some embodiments, the disease is prostate cancer, pancreatic cancer, or breast cancer. In some embodiments, the disease is an oncological, cardiovascular, renal, metabolic, or respiratory disease.

In certain embodiments, the disease is a lung disease or a central nervous system disease. In more specific embodiments, the disease is metastatic castration-resistant prostate cancer or metastatic breast cancer. In some embodiments, the method comprising administering to a subject in need thereof a therapeutically effective amount of a compound according to any one of the embodiments disclosed herein or a composition according to any one of the embodiments disclosed herein, wherein each M is independently a biologically active moiety effective for treating the disease.

In some embodiments, the disease is cancer, and each M is independently an anti-cancer drug. In some embodiments, at least one occurrence of M has one of the following structures:

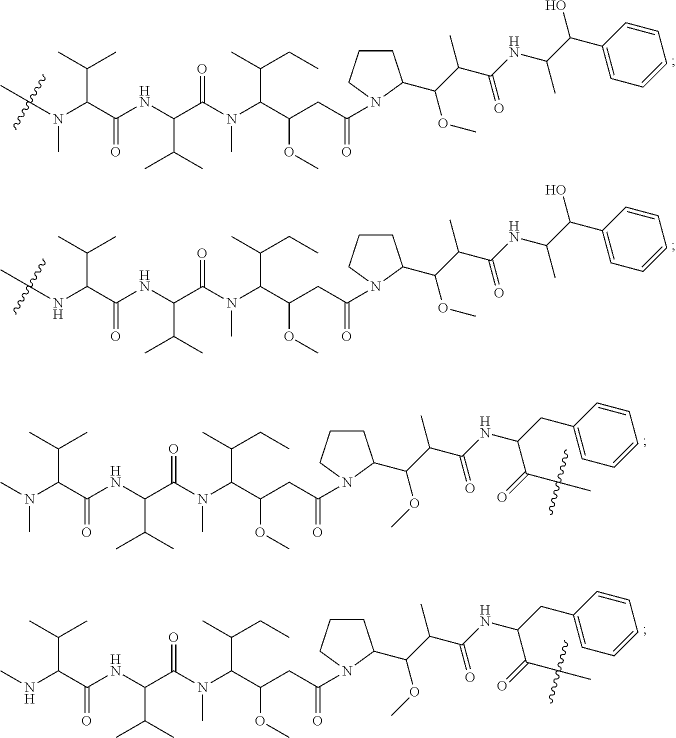 [SMILES: *N(C)C(C(=O)NC(C(=O)N(C)C(C(C)CC)C(CC(=O)N1CCCC1C(OC)C(C)C(=O)NC(C)C(O)c1ccccc1)OC)C(C)C)C(C)C ; *NC(C(=O)NC(C(=O)N(C)C(C(C)CC)C(CC(=O)N1CCCC1C(OC)C(C)C(=O)NC(C)C(O)c1ccccc1)OC)C(C)C)C(C)C ; *C(=O)C(Cc1ccccc1)NC(=O)C(C)C(OC)C1CCCN1C(=O)CC(OC)C(C(C)CC)N(C)C(=O)C(NC(=O)C(C(C)C)N(C)C)C(C)C ; *C(=O)C(Cc1ccccc1)NC(=O)C(C)C(OC)C1CCCN1C(=O)CC(OC)C(C(C)CC)N(C)C(=O)C(NC(=O)C(NC)C(C)C)C(C)C]

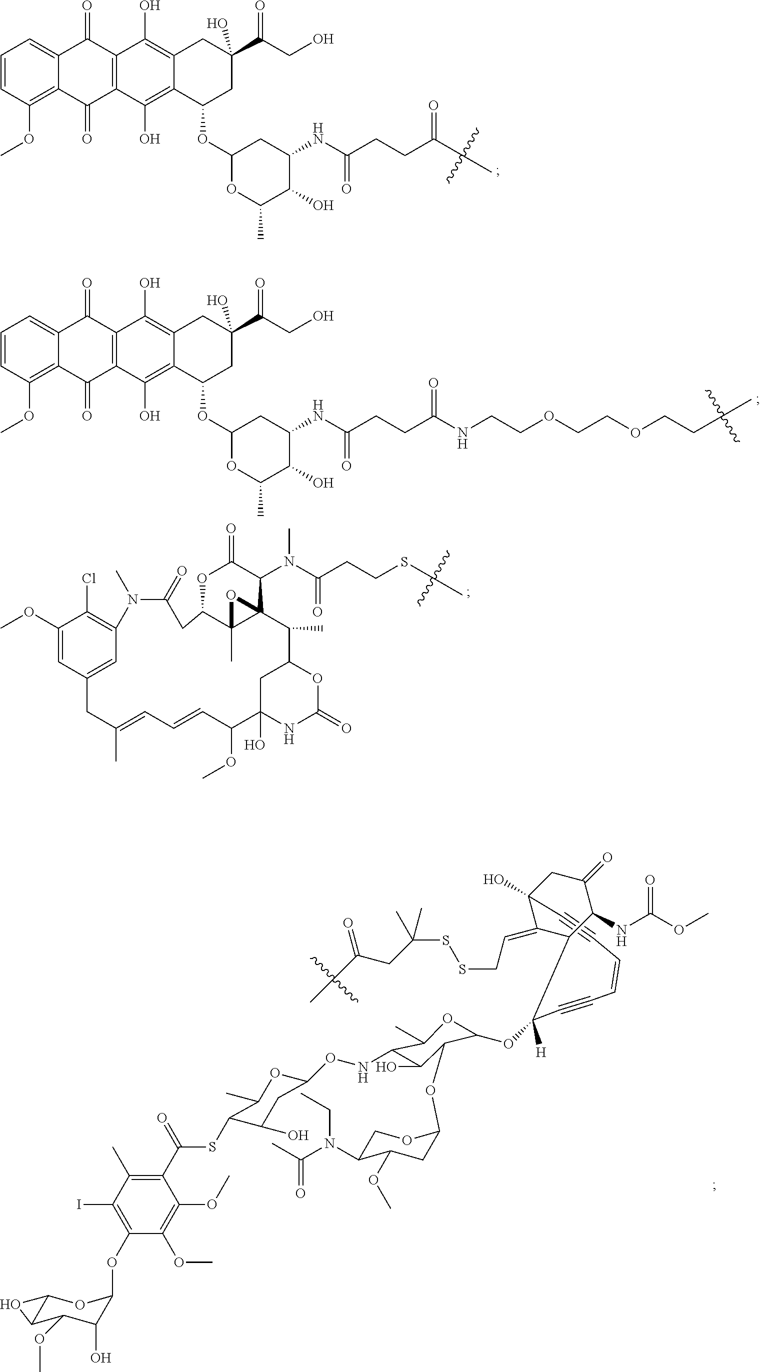

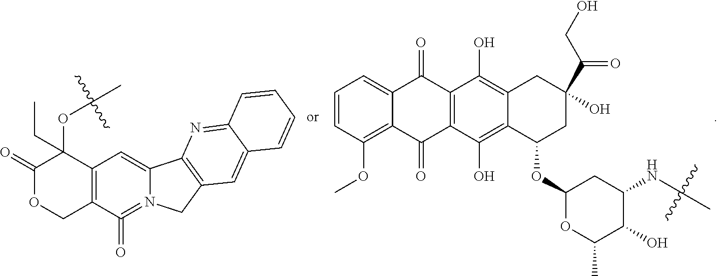

In some embodiments, each occurrence of M has one of the following structures:

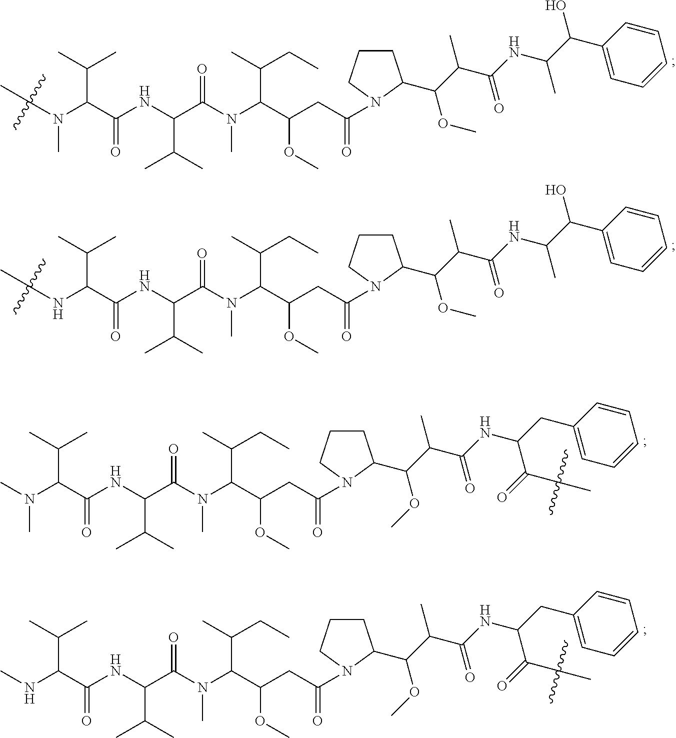 [SMILES: *N(C)C(C(=O)NC(C(=O)N(C)C(C(C)CC)C(CC(=O)N1CCCC1C(OC)C(C)C(=O)NC(C)C(O)c1ccccc1)OC)C(C)C)C(C)C ; *NC(C(=O)NC(C(=O)N(C)C(C(C)CC)C(CC(=O)N1CCCC1C(OC)C(C)C(=O)NC(C)C(O)c1ccccc1)OC)C(C)C)C(C)C ; *C(=O)C(Cc1ccccc1)NC(=O)C(C)C(OC)C1CCCN1C(=O)CC(OC)C(C(C)CC)N(C)C(=O)C(NC(=O)C(C(C)C)N(C)C)C(C)C ; *C(=O)C(Cc1ccccc1)NC(=O)C(C)C(OC)C1CCCN1C(=O)CC(OC)C(C(C)CC)N(C)C(=O)C(NC(=O)C(NC)C(C)C)C(C)C]

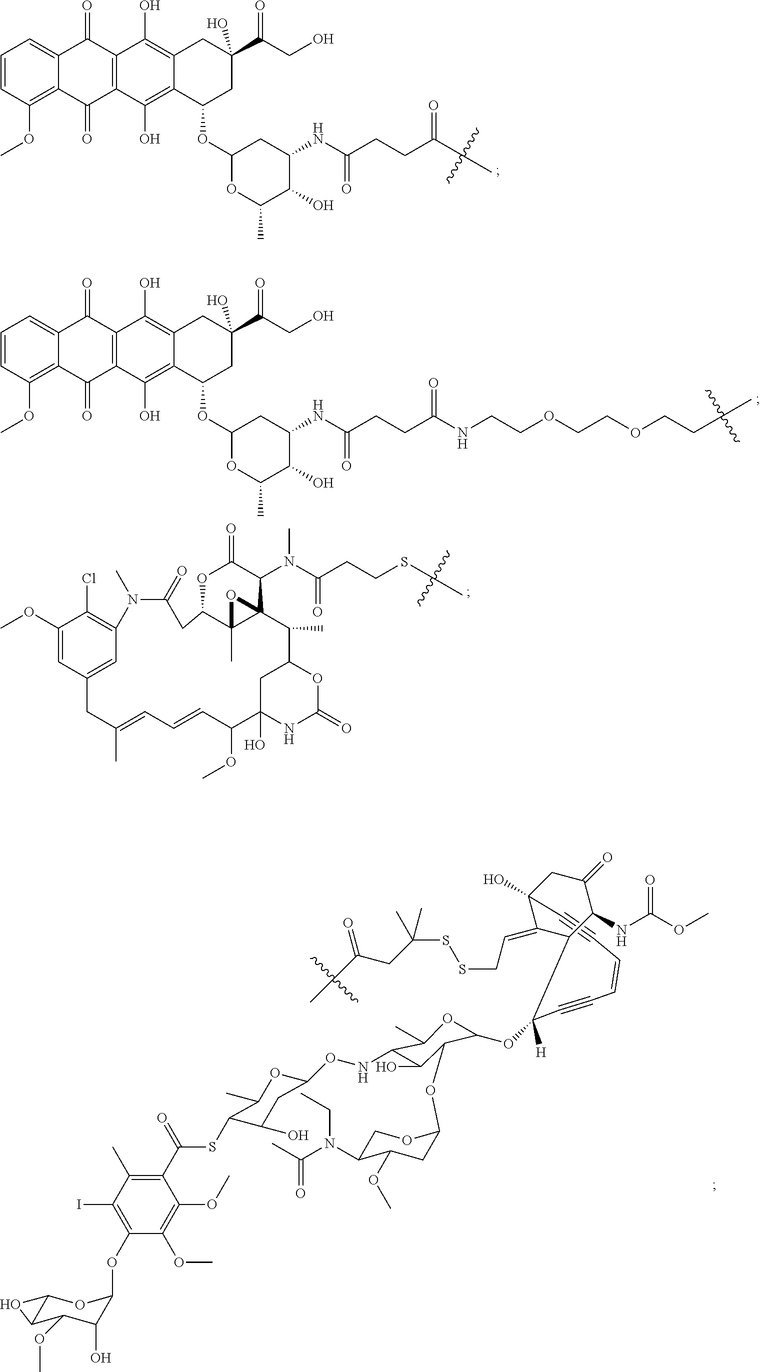

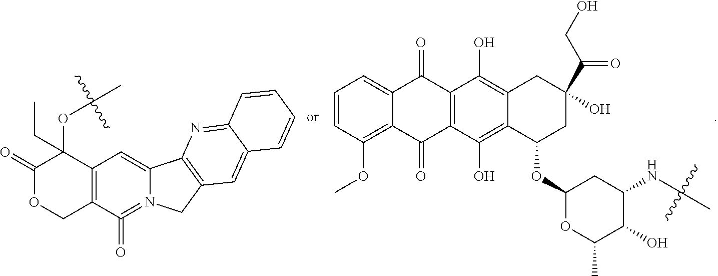

For example, in certain embodiments the disclosure provides a method of treating solid tumors, multiple myeloma, gliomas, clear cell renal cell carcinoma, prostate cancer, ovarian cancer, non-small cell lung cancer, GI malignancies, acute lymphoblastic leukemia, acute myelogenous leukemia, renal cell carcinoma, colorectal carcinoma, epithelial cancers, pancreatic and gastric cancers, renal cell carcinoma, non-Hodgkin's lymphoma, metastatic renal cell carcinoma, malignant mesothelioma, pancreatic, ovarian, and/or lung adenocarcinoma, B-cell malignancies, breast cancer, melanoma, recurrent multiple myeloma, small cell lung cancer, CD22-positive B cell malignancies, Hodgkin's lymphoma/anaplastic large cell lymphoma, or HER2-positive breast cancer.

In some of the foregoing embodiments, the disease is cancer. For example, in certain embodiments, the cancer is breast cancer, non-Hodgkin's lymphoma, acute myeloid leukemia, multiple myeloma, gastric cancer, renal cell carcinoma, solid tumors, ovarian cancer, prostate cancer, colorectal cancer, pancreatic cancer, small cell lung cancer, diffuse large B-cell lymphoma, a neoplasm, urothelial cancer, ALL, CLL, glioblastoma, Hodgkin's lymphoma, lymphoma, mesothelioma, non-small cell lung cancer, recurrent head and neck cancer, or a combination thereof.

Certain embodiments also relate to a method of treating a hyperproliferative disorder in a mammal (e.g., a human) that comprises administering to said mammal a therapeutically effective amount of a compound, or a pharmaceutically acceptable salt, ester, prodrug, solvate, hydrate or derivative thereof. In some embodiments, said method relates to the treatment of cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

Certain particular embodiments provide methods for treatment of lung cancers, the methods comprise administering an effective amount of any of the above described compounds (or a pharmaceutical composition comprising the same) to a subject in need thereof. In certain embodiments the lung cancer is a non-small cell lung carcinoma (NSCLC), for example adenocarcinoma, squamous-cell lung carcinoma or large-cell lung carcinoma. In other embodiments, the lung cancer is a small cell lung carcinoma. Other lung cancers treatable with the disclosed compounds include, but are not limited to, glandular tumors, carcinoid tumors and undifferentiated carcinomas.

Accordingly, in some embodiments of the foregoing methods, R² is a linker comprising a covalent linkage to a targeting moiety, such as an antibody, a cell surface receptor agonist, a cell surface receptor antagonist or the like. For example, epidermal growth factor receptor (EGFR) inhibitor, a hepatocyte growth factor receptor (HGFR) inhibitor, an insulin-like growth factor receptor (IGFR) inhibitor, a folate, or a MET inhibitor.

In even more embodiments, the method further comprises inducing apoptosis.

In some embodiments, the method for treating a disease further comprises:

(a) providing a compound of structure (I), for example, wherein one of R² or R³ is a linker comprising a covalent bond to an analyte molecule, and the other of R² or R³ is H, OH, alkyl, alkoxy, alkylether or —OP(═Rₐ)(R_{b})R_{c}; and

(b) detecting the compound by its visible properties.

In some embodiments the analyte molecule is a nucleic acid, amino acid or a polymer thereof (e.g., polynucleotide or polypeptide). In still more embodiments, the analyte molecule is an enzyme, receptor, receptor ligand, antibody, glycoprotein, aptamer or prion.

In certain embodiments, the providing further comprises admixing a compound of structure (I) with an analyte molecule.

Embodiments of the present compounds thus find utility in any number of methods, including, but not limited: drug delivery; quantifying apoptosis; qualifying therapeutic drug delivery; quantifying apoptosis; and diagnosing and treating diseases, such as blood cancers.

In addition to the above methods, embodiments of the compounds of structure (I) find utility in various disciplines and methods, including but not limited to:

cancer treatment and imaging, for example by including a targeting moiety, such as an antibody or sugar or other moiety that preferentially binds cancer cells, in a compound of structure (I) to; and/or drug delivery.

In some embodiments, the method of treatment comprises treating a tumor having tumor cells with tumor cell receptors. In some embodiments, the tumor cells have receptors ranging from 1,000 to 100,000, from 1,000 to 50,000, from 1,000 to 25,000 receptors, 1,000 to 10,000 receptors per cell. For example, in some embodiments the tumor cells have about 1,000, about 10,000, or less than 100,000 receptors per cell.

Embodiments of the present disclosure are not limited to treatment of cancer. In fact, the disclosures are not particularly limited with respect to the types of diseases, symptoms, conditions, indications, and treatments for which compounds and methods the present disclosure can be adapted. That is, the present disclosure provides compounds, compositions, and methods for treating or preventing a wide variety of diseases. For example, the compounds and compositions disclosed herein can be modified by selecting an appropriate biological moiety or combination of biological moieties to treat a particular disease as desired. Based on the present disclosure, it will be readily apparent to one of ordinary skill in the art how to modify the compounds and compositions presently disclosed in order to treat, prevent or target a disease, symptom or clinical indication.

Accordingly, methods of the present disclosure include methods of administering a compound of the present disclosure for treating diseases, conditions, or symptoms of a disease or condition, preventing diseases or symptoms of a disease or condition, prophylactically treating diseases, conditions or symptoms of a disease or condition, identifying a subject at risk and treating diseases, conditions, or symptoms of a disease or condition, slowing or stopping progression of diseases, conditions, or symptoms of a disease or condition, increasing the survival rate of a subject having diseases, conditions, or symptoms of a disease or condition, ameliorating the symptoms of diseases, conditions, or symptoms of a disease or condition, and the like. Additionally, the diseases, conditions, symptoms, afflictions, side effects, maladies, syndromes, biological occurrences, biological abnormalities, medical conditions, illnesses, pathosis, pathology, and the like are meant to be included within the present disclosure and are also not particularly limited; examples include, but are not limited to, cancer, inflammation, pain, pain control, inflammatory diseases, infectious diseases, viral infections, genetic disorders, bacterial infections, fungal infections, cutaneous conditions, endocrine conditions, ocular disorders, intestinal disease, neurological disorders, liver disorders, lung infections, heart conditions and disorders, mental illness (e.g., eating disorders, mood disorders, personality disorders), norovirus infections, blood-borne pathogens, protozoan infections, viral hepatitis, HIV/AIDS, diabetes, sclerosis, Crohn's and colitis, lupus, arthritis, allergies and asthma, celiac disease, polychondritis, scleroderma, liver disease, heart disease, acquired diseases, acute diseases, chronic conditions or diseases, congenital diseases or disorders, hereditary diseases or disorders, iatrogenic disease, idiopathic disease, primary disease, secondary disease, terminal disease, or similar. The foregoing may be acute, chronic, clinical, a flare-up, progressive, refractory, subclinical, localized, disseminated, systemic, or the like. Any of the foregoing examples may include being caused as a result of an airborne, foodborne, infections, or lifestyle event.

In some embodiments, the biologically active moiety is an antibiotic drug. In certain specific embodiments, M is at each occurrence, independently an antibiotic drug, and the targeting moiety is an antibody specific for an infectious disease antigen. Antibiotic drug, as used herein, includes derivatives. That is, an antibiotic drug that has been modified or derivatized such that the drug can be conjugated or attached to another molecule (e.g., to include Q moieties).

Exemplary anti-biotic moieties may include compounds to treat a bacterial species including, for example, Actinomyces israelii, Bacillus anthracis, Bacteroides fragilis, Bordetella pertussis, Borrelia sp., Brucella sp., Campylobacter jejuni, Chlamydia sp., Chlamydophila psittaci, Clostridium sp., Corynebacterium diphtheria, Ehrlichia sp., Enterococcus sp., Escherichia sp., Francisella tularensis, Haemophilus influenza, Helicobacter pylori, Klebsiella pneumoniae, Legionella pneumophila, Leptospira sp., Listeria monocytogenes, Mycobacterium sp., Mycoplasma pneumoniae, Neisseria sp., Pseudomonas aeruginosa, Nocardia asteroids, Rickettsia rickettsia, Salmonella sp., Shigella sp., Staphylococcus sp., Streptococcus sp., Treponema pallidum, Vibrio cholera, and Yersinia pestis. Further, bacterial species that can be treated include drug resistant strains, such as methicillin resistant Staphylococcus aureus (MRSA), vancomycin resistant Enterococci (VRE), and various multi-drug resistant (MDR) strains commonly associated with hospital-acquired (nosocomial) infections, including Acinetobacter baumannii, Klebsiella pnuemoniae, and Enterobacter cloacae. In another embodiment, the bacterial diseases or conditions that can be treated with compounds of structure (I) can be, for example, Anthrax, Whooping cough, Lyme disease, Brucellosis, gastrointestinal abscesses, relapsing fever, enteritis, bloody diarrhea, atypical pneumonia, botulism, tetanus, bacterial meningitis, gangrene, bacterial endocarditis, leprosy, Legionnaire's disease, leptospirosis, tuberculosis, plague, cholera, necrotizing fasciitis, typhoid fever, and nocardiosis.

In some more specific embodiments, the antibiotic drug is a β-lactam antibiotic. β-lactam antibiotics include compounds comprising penicillins, monobactams, carbapenems and cephalosporins. In some embodiments, M is oxacillin, dicloxacillin, nafcillin, amoxicillin, ampicillin, piperacillin, Cloxacillin, Flucloxacillin, Methicillin, Oxacillin, Temocillin, Benzylpenicillin (penicillin G), Almecillin (penicillin O), Phenoxymethylpenicillin (penicillin V), Mecillinam, Carbenicillin, Ticarcillin, Azlocillin, Mezlocillin, Cefazolin, Cephalexin, Cephalosporin C, Cefotaxime, Cefdinir, Cefpirome, Biapenem, Doripenem, Ertapenem, Faropenem, Imipenem, Meropenem, Panipenem, Razupenem, Tebipenem, Thienamycin, Aztreonam, Tigemonam, Nocardicin A, Tabtoxinine β-lactam, Lenapenem, Tomopenem, Cefazolinm Cefalexin, Cefadroxil, Cefapirin, Cefazedone, Cefazaflur, Cefradine, Cefroxadine Ceftezole, Cefaloglycin, Cefacetrile, Cefalonium, Cefaloridine, Cefalotin, Cefatrizine, Cefaclor, Cefotetan, Cephamycin, Cefoxitin, Cefprozil, Cefuroxime, Cefuroxime axetil, Cefamandole, Cefminox, Cefonicid, Ceforanide, Cefotiam, Cefbuperazone, Cefuzonam, Cefmetazole, Carbacephem (Loracarbef), Cefixime, Ceftriaxone, Ceftazidime, Cefoperazone Cefdinir, Cefcapene, Cefdaloxime, Ceftizoxime, Cefmenoxime, Cefotaxime, Cefpiramide, Cefpodoxime, Ceftibuten, Cefditoren, Cefetamet, Cefodizime, Cefpimizole, Cefsulodin, Cefteram, Ceftiolene, Oxacephem, Flomoxef, Cefepime, Cefozopran, Cefpirome, Cefquinome, Ceftaroline fosamil, Ceftolozane, Ceftobiprole, Ceftiofur, Cefquinome, or Cefovecin for at least one occurrence of M or at each occurrence of M.

In other embodiments, the antibiotic drug is a tetracycline antibiotic. More specifically, in some embodiments, M is Doxycycline, tetracycline, minocycline, demeclocycline, Chlortetracycline, Oxytetracycline, Lymecycline, Methacycline, Rolitetracycline, Tigecycline, Eravacycline, Sarecycline, Omadacycline, Clomocycline, Demeclocycline, Meclocycline, Metacycline, or Penimepicycline for at least one occurrence of M or at each occurrence of M.

In further embodiments, the antibiotic drug is a quinolone antibiotic. Quinolone antibiotics comprise the large sub-group fluoroquinolones. In particular, in some embodiments, M is oxolinic acid (Uroxin), rosoxacin (Eradacil), ciprofloxacin (Zoxan, Ciprobay, Cipro, Ciproxin), fleroxacin (Megalone, Roquinol), lomefloxacin (Maxaquin), nadifloxacin (Acuatim, Nadoxin, Nadixa), norfloxacin (Lexinor, Noroxin, Quinabic, Janacin), ofloxacin (Floxin, Oxaldin, Tarivid), pefloxacin (Peflacine), rufloxacin (Uroflox), balofloxacin (Baloxin), grepafloxacin (Raxar), levofloxacin (Cravit, Levaquin), pazufloxacin (Pasil, Pazucross), sparfloxacin (Zagam), temafloxacin (Omniflox), clinafloxacin, gatifloxacin (Zigat, Tequin) (Zymar-opth.), moxifloxacin (Avelox, Vigamox), sitafloxacin (Gracevit), prulifloxacin, besifloxacin (Besivance), delafloxacin (Baxdela), gemifloxacin (Factive), ozenoxacin, tosufloxacin, cinoxacin (Cinobac), nalidixic acid (NegGam, Wintomylon), piromidic acid (Panacid), pipemidic acid (Dolcol), nemonoxacin, or enoxacin for at least one occurrence of M or at each occurrence of M.

In yet another embodiment, the antibiotic drug is a lincosamide antibiotic. In some embodiments, M is Clindamycin, Lincomycin, or Pirlimycin for at least one occurrence of M or at each occurrence of M.

In some embodiments, the antibiotic drug is a macrocyclic antibiotic. Macrocyclic antibiotics comprise groups including macrolides, ketolides, fluoroketolides, and polyenes. In some embodiments, M is Amphotericin B, Azithromycin, Boromycin, Carbomycin A, Cethromycin, Clarithromycin, Dirithromycin, Erythromycin, Fidaxomicin, Flurithromycin, Josamycin, Kitasamycin, Midecamycin, Miocamycin, Oleandomycin, rifampicin (or rifampin), rifabutin, rifapentine, rifalazil, rifaximin, Rifamycin SV, Rokitamycin, Roxithromycin, Solithromycin, Spiramycin, Telithromycin, Troleandomycin, or Tylosin for at least one occurrence of M or at each occurrence of M.

In some embodiments, the antibiotic drug is a sulfonamide antibiotic (sulfa or sulpha drugs). Sulfonamide antibiotics exert their bacterostatic effect through inhibition of dihydropteroate synthase (DHPS) thereby interrupting folate synthesis and the ability of the organism to synthesize nucleic acids. In some embodiments, M is Sulfafurazole, Sulfacetamide, Sulfadiazine, Sulfadimidine, Sulfafurazole (sulfisoxazole), Sulfisomidine (sulfaisodimidine), Sulfanitran, Sulfadimethoxine, Sulfamethoxypyridazine, Sulfametoxydiazine, Sulfadoxine, Sulfametopyrazine, Terephtyl, Sulfamethoxazole, or Sulfamoxole for at least one occurrence of M or at each occurrence of M.

In some embodiments, the antibiotic drug is a glycopeptide antibiotic. In certain embodiments, M is vancomycin, teicoplanin, telavancin, ramoplanin, oritavancin or decaplanin for at least one occurrence of M or at each occurrence of M.

In some embodiments, the antibiotic drug is an aminoglycoside antibiotic. Aminoglycoside antibiotics exert their biological effect through protein synthesis inhibition. In more specific embodiments, M is Streptomycin, Dihydrostreptomycin, Neomycin, Framycetin, Paromomycin, Ribostamycin, Kanamycin, Amikacin, Arbekacin, Bekanamycin, Dibekacin, Tobramycin, Spectinomycin, Hygromycin B, Apramycin, Puromycin, Nourseothricin, Gentamicin, Netilmicin, Sisomicin, Plazomicin, Isepamicin, Verdamicin, or Astromicin for at least one occurrence of M or at each occurrence of M.

In some embodiments, the antibiotic drug is an oxazolidinone antibiotic. Oxazolidinone antibiotics exert their biological effect through protein synthesis inhibition. In some specific embodiments, Eperezolid, Linezolid, Posizolid, Radezolid, Ranbezolid, Sutezolid, or Tedizolid for at least one occurrence of M or at each occurrence of M.

In some embodiments, M is platensimycin, chloramphenicol, metronidazole, trimethoprim, aditoprim, brodimoprim, clofazimine, iclaprim, tetroxoprim, or nitrofurantoin for at least one occurrence of M or at each occurrence of M.

In some embodiments, the biologically active moiety is an antifungal drug. In certain specific embodiments, M is at each occurrence, independently an antifungal drug, and the targeting moiety is an antibody specific for an infectious disease antigen. Antifungal drug, as used herein, includes derivatives. That is, an antifungal drug that has been modified or derivatized such that the drug can be conjugated or attached to another molecule (e.g., to include Q moieties).

Fungal species that can be treated with compounds of the present disclosure include, for example; Candida sp., Aspergillus sp., Cryptococcus sp., Histoplasma sp., Pneumocystis sp., and Stachybotrys sp. Further, fungal species necessitating treatment include emerging drug resistant strains, such as Candida auris, glabrata and krusei which demonstrate significant resistance to existing treatment options and present a public health concern since they are frequently health center acquired infections.

In some embodiments, the fungal diseases or conditions that can be treated with compounds of structure (I) can be, for example aspirgillosis, invasive candidiasis, onychomycosis or histoplasmosis for at least one occurrence of M or at each occurrence of M.

In certain embodiments, the antifungal drug is a polyene. In more specific embodiments, M is amphotericin B, candicidin, filipin, hamycin, natamycin, nystatin, and rimocidin; an allylamine such as amorolfin, butenafine, naftifine, and terbinafine; an echinocandin such as anidulafungin, caspofungin, and micafungin; or azoles, which can be divided into imidazoles such as bifonazole, butoconazole, clotrimazole, econazole, fenticonazole, isoconazole, ketoconazole, luliconazole, miconazole, omoconazole, oxiconazole, sertaconazole, sulconazole, and tioconazole; triazoles such as albaconazole, efinaconazole, epoxiconazole, fluconazole, isavuconazole, itraconazole, posaconazole, propiconazole, ravuconazole, terconazole, and voriconazole; and the thiazole, abafungin for at least one occurrence of M or at each occurrence of M.

In some more specific embodiments, M is ciclopirox, flucytosine or 5-fluorocytosine, griseofulvin, tolnaftate, orotomide, miltefosine, piroctone olamine, iodoquinol, clioquinol, acrisorcin, or fumagillin for at least one occurrence of M or at each occurrence of M.

In some embodiments, the biologically active moiety is an antiparasitic drug. In certain specific embodiments, M is at each occurrence, independently an antiparasitic drug, and the targeting moiety is an antibody specific for an infectious disease antigen. Antiparasitic drug, as used herein, includes derivatives. That is, an antiparasitic drug that has been modified or derivatized such that the drug can be conjugated or attached to another molecule (e.g., to include Q moieties).

Parasitic organisms with their associated diseases that can be treated with compounds of the present disclosure include protists such as Plasmodium sp. (malaria), Leishmania sp. (leishmaniasis), Trypanosoma sp. (African trypanosomiasis/sleeping sickness, Chagus disease), Giardia sp. (giardiasis/beaver fever), Toxoplasma gondii (toxoplasmosis), and Cryptosporidium sp. (cryptosporidiosis); and amobae such as Entamoeba histolytica (amoebiasis).

In certain embodiments, M is chloroquine, amodiaquine, pyrimethamine (Daraprim), proguanil, sulfadoxine, sulfamethoxypyridazine, mefloquine, paromomycin, atovaquone, primaquine, artemisinin, amphotericin B, artemether, artesunate, dihydroartemisinin, arteether, doxycycline, clindamycin, halofantrine, diloxanide, eflornithine, furazolidone, melarsoprol, metronidazole, nifursemizone, nitazoxanide, ornidazole, paromomycin sulfate, pentamidine, pyrimethamine, quinapyramine or tinidazole for at least one occurrence of M or at each occurrence of M.

In some embodiments, the biologically active moiety is an antiviral drug. In certain specific embodiments, M is at each occurrence, independently an antiviral drug, and the targeting moiety is an antibody specific for an infectious disease antigen. Antiviral drug, as used herein, includes derivatives. That is, an antiviral drug that has been modified or derivatized such that the drug can be conjugated or attached to another molecule (e.g., to include Q moieties).

Viral diseases that can be treated with compounds of the present disclosure include, for example HIV, Zika, Ebola, hepatitis B and C, and influenza.

In some embodiments, M is pegylated interferon-alpha-2a/2b, entecavir, tenofovir disoproxil fumarate, asunaprevir, tribavirin, beclabuvir, daclatasvir, dasabuvir, grazoprevir, paritaprevir, simeprevir, sofosbuvir, or velpatasvir for at least one occurrence of M or at each occurrence of M.

In other embodiments, the antiviral drug treats HIV. In some embodiments, M is a nucleoside/nucleotide reverse transcriptase inhibitor such as abacavir, lamivudine, tenofovir disoproxil fumarate, and zidovudine; a non-nucleoside reverse transcriptase inhibitor such as efavirenz or nevirapine; a protease inhibitor such as atazanavir, darunavir, lopinavir, and ritonavir; or, an integrase inhibitor such as dolutegravir or raltegravir for at least one occurrence of M or at each occurrence of M.

In yet another embodiment, the antiviral drug treats influenza. For example, in some embodiments, M is laninamivir, oseltamivir, peramivir, zanamivir, or baloxavir marboxil for at least one occurrence of M or at each occurrence of M.

In some embodiments, the antiviral drug treats Ebola. For example, in some embodiments, M is favipiravir, brincidofovir, galidesivir (BCX4430, Immucillin-A), JK-05, or AVI-7537 for at least one occurrence of M or at each occurrence of M.

In some embodiments, the biologically active moiety is a drug for the treatment of immunologic or anti-inflammatory conditions. In certain specific embodiments, M is at each occurrence, independently an immunologic drug, and the targeting moiety is an antibody specific for a disease/condition related antigen. Immunologic drug, as used herein, includes derivatives. That is, an immunologic drug that has been modified or derivatized such that the drug can be conjugated or attached to another molecule (e.g., to include Q moieties).

In some embodiments, the disease or condition is Asthma, Rheumatoid arthritis, lupus, multiple sclerosis, psoriasis, Crohn's disease, colitis, or organ rejection therapy. Accordingly, in some embodiments, M is a corticosteroids (e.g., prednisone), methotrexate, mycophenolate mofetil, or azathioprine for at least one occurrence of M or at each occurrence of M. In some embodiments, the compound of structure (I) comprises a covalent bond to a therapeutic antibody such as Humira (i.e., adalimumab).

In some embodiments, the disease or condition is psoriasis. Accordingly, in some embodiments, M is a acitretin, prednisone, retinoids, methotrexate, cyclosporine, thioguanine, etanercept (Enbrel), infliximab (Remicade), adalimumab (Humira), ustekinumab (Stelara), golimumab (Simponi), apremilast (Otezla), secukinumab (Cosentyx), or ixekizumab (Taltz) for at least one occurrence of M or at each occurrence of M.

In some embodiments, the disease or condition is Crohn's disease or colitis. In some more specific embodiments M is a sulfasalazine, mesalamine, balsalazide, olsalazine, prednisone, hydrocortisone, mercaptopurine, azathioprine, cyclosporine, methotrexate, budesonide, ciprofloxacin, metronidazole, or azathioprine, for at least one occurrence of M or at each occurrence of M. In some embodiments, the compound of structure (I) comprises a covalent bond to a therapeutic antibody such as infliximab (Remicade), adalimumab (Humira), vedolizumab (Entyvio), certolizumab, natalizumab (Tysabri), ustenkinumab (Stelara), or golimumab (Simponi).

In some embodiments, the disease or condition is asthma. In some embodiments, M is a corticosteroid, long-acting beta agonist (e.g., salmeterol, leukotriene), omalizumab, zafirlukast, or fluticasone for at least one occurrence of M or at each occurrence of M.

In some embodiments, the disease or condition is multiple sclerosis. In some embodiments, M is a hydroxychloroquine, methotrexate, azathioprine, mycophenolate, prednisone, methylprednisolone, belimumab, or rituximab (Rituxan) for at least one occurrence of M or at each occurrence of M.

In some embodiments, the disease or condition is organ rejection. In more specific embodiments, M is a prednisolone, hydrocortisone, sirolimus, everolimus, cyclosporine, tacrolimus, mycophenolate, or azathioprine, basiliximab, daclizumab, or rituximab for at least one occurrence of M or at each occurrence of M.

In some embodiments, the disease or condition is lupus. In more specific embodiments, M is a hydroxychloroquine, methotrexate, azathioprine, mycophenolate, prednisone, methylprednisolone, belimumab, or rituximab (Rituxan) for at least one occurrence of M or at each occurrence of M.

In some embodiments, the disease or condition is rheumatoid arthritis.

In more specific embodiments, M is a prednisone, methotrexate, mycophenolate mofetil, leflunomide, hydroxychloroquine, sulfasalazine, azathioprine, abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) or tofacitinib (Xeljanz) for at least one occurrence of M or at each occurrence of M.

In some embodiments, the disease or condition to be treated by administering a compound of structure (I) is ablation of bone marrow; acute sciatic pain; allergic asthma; ALS and multiple sclerosis; Alzheimer's disease; amyloidosis; angioedema; angiogenesis; angiosarcoma; ankylosing spondylitis; anthrax (prophylaxis and treatment); arthritis; asthma; atopic diseases; atypical hemolytic uremic syndrome; autoimmune hepatitis; Bacillus anthracis spores; B-cell malignancies; cardiovascular disease; choroidal and retinal neovascularization; chronic asthma; chronic hepatitis B; clinical signs of atopic dermatitis in dogs; Clostridium difficile colitis; cold agglutinin disease; Crohn's disease; Cryopyrin-associated periodic syndrome; cytomegalovirus infection; diabetes; diabetic nephropathy and arteriovenous graft patency; Duchenne muscular dystrophy; dyslipidemia; Ebola virus; eczema; fibrosis; geographic atrophy secondary to age-related macular degeneration; glioblastoma; graft versus host disease; haemophilia A; haemorrhagic shock; heart attack, stroke, traumatic shock; hematologic malignancies; hemolytic disease of the newborn; hepatitis B; HIV infection; hypercholesterolemia; immunologically mediated inflammatory disorders; infectious disease/influenza A; inflammation; inflammations of the airways, skin and gastrointestinal tract; inflammatory bowel disease; invasive Candida infection; juvenile idiopathic arthritis; lupus nephritis; macular degeneration (wet form); medically attended lower respiratory disease; melanoma; migraine; multiple sclerosis; muscle atrophy due to orthopedic disuse and sarcopenia; muscle wasting disorders; muscular dystrophy; myostatin inhibitor; Neovascular age-related macular degeneration; neuromyelitis optica; nosocomial pneumonia; ocular vascular diseases; oncology/immune indications; organ transplant rejection; osteoarthritis; osteomyelitis (imaging); osteoporosis; osteoporosis, bone metastases etc.; Parkinson's disease; paroxysmal nocturnal hemoglobinuria; percutaneous coronary intervention; Plaque psoriasis; platelet aggregation inhibitor; post-exposure prophylaxis of rabies; prevention of organ transplant rejections; primary systemic amyloidosis; progressive supranuclear palsy; Pseudomonas aeruginosa infection; psoriasis; psoriatic arthritis; rabies (prophylaxis); recovery of motor function after stroke; recurrent glioblastoma multiforme; reduction of scarring after glaucoma surgery; reduction of side effects of cardiac surgery; respiratory syncytial virus; reversal of anticoagulant effects of dabigatran; Rh disease; rheumatic diseases; rheumatoid arthritis; sepsis; severe allergic disorders; severe asthma and chronic spontaneous urticaria; sickle-cell disease; SLE, dermatomyositis, polymyositis; solid malignancies; Staphylococcus aureus infection; systemic lupus erythematosus; systemic scleroderma; thromboembolism (diagnosis); thrombotic thrombocytopenic purpura, thrombosis; thyroid eye disease; TNF; ulcerative colitis; uveitis, rheumatoid arthritis psoriasis; viral infections; wet age-related macular degeneration; white blood cell disease; X-linked hypophosphatemia or combinations thereof.

Further therapeutic agents that can be combined with a compound of the disclosure are found in Goodman and Gilman's “The Pharmacological Basis of Therapeutics” Tenth Edition edited by Hardman, Limbird and Gilman or the Physician's Desk Reference, both of which are incorporated herein by reference in their entirety.

The compounds described herein can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some embodiments of the separate administration protocol, a compound of the disclosure and any of the agents described above are administered a few minutes apart, or a few hours apart, or a few days apart.

In some embodiments, the method further comprises administering an additional therapeutic agent selected from the group consisting of an antineoplastic agent, an enediyne antitumor antibiotic, a maytansinoid, a topoisomerase inhibitor, a kinase inhibitor, an anthracycline, and EGFR inhibitor, an alkylating agent and combinations thereof.

In some more specific embodiments, the method further comprises administering an additional therapeutic agent selected from the group consisting of an antineoplastic agent, an enediyne antitumor antibiotic, a maytansinoid, a topoisomerase inhibitor, a kinase inhibitor, an anthracycline, and EGFR inhibitor, an alkylating agent and combinations thereof.

In certain embodiments, the additional therapeutic agent comprises auristatin F, monomethyl auristatin F, monomethyl auristatin E, paciltaxol, SN-38, calicheamicin, anthramycin, abbeymycin, chicamycin, DC-81, mazethramycin, neothramycin A, neothramycin B, porothramycin prothracarcin, sibanomicin, sibiromycin, tomamycin, mertansine, emtansine, irinotecan, camptothecin, topotecan, silatecan, cositecan, Exatecan, Lurtotecan, gimatecan, Belotecan, and Rubitecan.

The examples and preparations provided below further illustrate and exemplify the compounds of the present disclosure and methods of preparing such compounds. It is to be understood that the scope of the present disclosure is not limited in any way by the scope of the following examples and preparations. In the following examples, and throughout the specification and claims, molecules and moieties with a single stereocenter, unless otherwise noted, exist as a racemic mixture. Those molecules and moieties with two or more stereocenters, unless otherwise noted, exist as a racemic mixture of diastereomers. Single enantiomers/diastereomers may be obtained by methods known to those skilled in the art.

### Methods of Preparation

Numerous advantages are afforded by embodiments disclosed herein, including the ability to control the number of biologically active moieties or fluorescent dye moieties that are coupled to the polymer and any subsequent targeting moiety. The composition of the polymer backbone can also be selected to afford desirable solubility properties, for example, by controlling the incorporation of charged moieties (e.g., number, frequency, spacing, etc.). In addition to the properties provided by the composition of the backbone, the side chains can be selected to provide a source for tuning the solubility of the compounds disclosed herein.

The embodiments disclosed herein also provide compounds that can advantageously include multiple therapeutic agents, for example, for complimentary or synergistic therapeutic strategies. In addition, embodiments of the present disclosure provide combinations of therapeutic agents, targeting moieties, and dye moieties (e.g., fluorophores) that can be used for simultaneous targeting, treatment and detection. The ease of coupling polymer-drug constructs to targeting agents such as antibodies, antibody fragments, proteins or other clinically interesting agents provides utility to a wide variety of interesting applications (e.g., surface chemistries, assay development, etc.)

The compounds of certain embodiments also provide other desirable properties, including enhanced permeability and retention effects. In addition to providing necessary solubility characteristics, the chemical features of embodiments of the present compounds can be adjusted to modulate the compound's ability to permeate diseased cells/tissue and be retained within the same. These features allow effective delivery of biologically active agents by increasing permeation and increasing efficacy by enhancing retention.

Accordingly, it is understood that any embodiment of the compounds of structure (I), as set forth above, and any specific choice set forth herein for a R¹, R², R³, R⁴, R⁵, L^{a}, L^{b}, L¹, L², L³, M, m and/or n variable in the compounds of structure (I), as set forth above, may be independently combined with other embodiments and/or variables of the compounds of structure (I) to form embodiments of the disclosure not specifically set forth above. In addition, in the event that a list of choices is listed for any particular R¹, R², R³, R⁴, R⁵, L^{a}, L^{b}, L¹, L², L³, M, m and/or n variable in a particular embodiment and/or claim, it is understood that each individual choice may be deleted from the particular embodiment and/or claim and that the remaining list of choices will be considered to be within the scope of the disclosure.

It is understood that in the present description, combinations of substituents and/or variables of the depicted formulae are permissible only if such contributions result in stable compounds.

It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

Furthermore, all compounds of the disclosure which exist in free base or acid form can be converted to their salts by treatment with the appropriate inorganic or organic base or acid by methods known to one skilled in the art. Salts of the compounds of the disclosure can be converted to their free base or acid form by standard techniques.

The following Reaction Schemes illustrate exemplary methods of making compounds of this disclosure. It is understood that one skilled in the art may be able to make these compounds by similar methods or by combining other methods known to one skilled in the art. It is also understood that one skilled in the art would be able to make, in a similar manner as described below, other compounds of structure (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described in this disclosure.

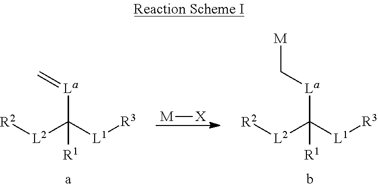

Reaction Scheme I illustrates an exemplary method for preparing an intermediate useful for preparation of compounds of structure (I), where R¹, L¹, L² and M are as defined above, R² and R³ are as defined above or are protected variants thereof and L^{a} is an optional linker. Referring to Reaction Scheme 1, compounds of structure a can be purchased or prepared by methods well-known to those of ordinary skill in the art. Reaction of a with M-X, where X is a halogen such as bromo, under Suzuki coupling conditions known in the art results in compounds of structure b. Compounds of structure b can be used for preparation of compounds of structure (I) as described below.

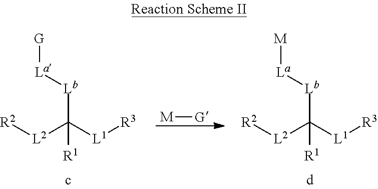

Reaction Scheme II illustrates an alternative method for preparation of intermediates useful for preparation of compounds of structure (I). Referring to reaction Scheme II, where R¹, L^{a}, L^{b}, L¹, L² and M are as defined above, and R² and R³ are as defined above, or are protected variants thereof, a compound of structure c, which can be purchased or prepared by well-known techniques, is reacted with M-G′ to yield compounds of structure d. Here, G and G′ represent functional groups having complementary reactivity (i.e., functional groups which react to form a covalent bond). G′ may be pendant to M or a part of the structural backbone of M. L^{a′} is an intermediate selected such that the reaction depicted above converts L^{a′} to L^{a}. G and G′ may be any number of functional groups described herein, such as alkyne and azide, respectively, amine and activated ester, respectively or amine and isothiocyanate, respectively, and the like.

The compound of structure (I) may be prepared from one of structures b or d by reaction under well-known automated DNA synthesis conditions with a phosphoramidite compound having the following structure (e):

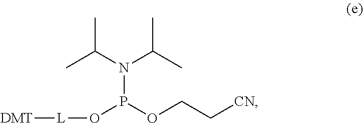

wherein each L is independently an optional linker.

DNA synthesis methods are well-known in the art. Briefly, two alcohol groups, for example R² and R³ in intermediates b or d above, are functionalized with a dimethoxytrityl (DMT) group and a 2-cyanoethyl-N,N-diisopropylamino phosphoramidite group, respectively. The phosphoramidite group is coupled to an alcohol group, typically in the presence of an activator such as tetrazole, followed by oxidation of the phosphorous atom with iodine. The dimethoxytrityl group can be removed with acid (e.g., chloroacetic acid) to expose the free alcohol, which can be reacted with a phosphoramidite group. The 2-cyanoethyl group can be removed after oligomerization by treatment with aqueous ammonia.

Preparation of the phosphoramidites used in the oligomerization methods is also well-known in the art. For example, a primary alcohol (e.g., R³) can be protected as a DMT group by reaction with DMT-Cl. A secondary alcohol (e.g., R²) is then functionalized as a phosphoramidite by reaction with an appropriate reagent such as 2-cyanoethyl N,N-dissopropylchlorophosphoramidite. Methods for preparation of phosphoramidites and their oligomerization are well-known in the art.

Compounds of structure (I) are prepared by oligomerization of intermediates b or d and e according to the well-known phophoramidite chemistry described above. The desired number of n repeating units is incorporated into the molecule by repeating the phosphoramidite coupling the desired number of times.

In certain embodiments compounds of structure (I) are prepared from one or more of the following phosphoramidites (e):

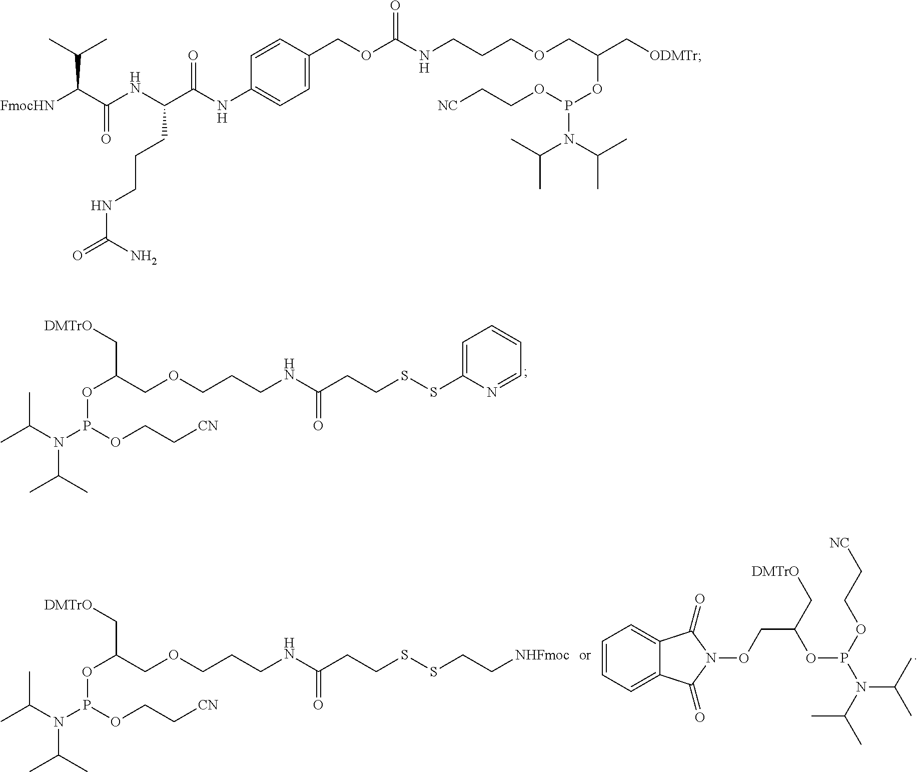

In exemplary embodiments, the G moiety can be selected from any of the Q moieties described herein, including those specific examples provided in Table 1. In some embodiments, G comprises, at each occurrence, independently a moiety suitable for reactions including: the copper catalyzed reaction of an azide and alkyne to form a triazole (Huisgen 1,3-dipolar cycloaddition), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse-demand Diels-Alder, alkene and tetrazole photoreaction and various displacement reactions, such as displacement of a leaving group by nucleophilic attack on an electrophilic atom.

In some embodiments, G is, at each occurrence, independently a moiety comprising an aldehyde, oxime, hydrazone, alkyne, amine, azide, acylazide, acylhalide, nitrile, nitrone, sulfhydryl, disulfide, sulfonyl halide, isothiocyanate, imidoester, activated ester, ketone, α,β-unsaturated carbonyl, alkene, maleimide, α-haloamide, epoxide, aziridine, tetrazine, tetrazole, phosphine, biotin or thiirane functional group.

In other embodiments, G comprises, at each occurrence, independently an alkyne or an azide group. In other embodiments, G comprises, at each occurrence, independently an amino, isothiocyanate or activated ester group. In different embodiments, G comprises, at each occurrence, independently a reactive group capable of forming a functional group comprising an alkene, ester, amide, thioester, disulfide, carbocyclic, heterocyclic or heteroaryl group, upon reaction with the complementary reactive group. For example, in some embodiment the heteroaryl is triazolyl.

In some embodiments, compounds of structure (I) are prepared by oligomerization of intermediates b or d and e according to phophoramidite chemistry described above, such that multiple, different linking groups (e.g., “L^{a} groups”) can be introduced which have multiple, different release mechanisms as described herein (for example by esterase, Cathepsin B, in vivo hydrolysis and the like) or are non-cleavable under physiological conditions. Further, these compounds can be modified to include one or more M moieties that are the same or different. Accordingly, compounds of structure (I) can be customized or “programmed” such that an M moiety is “released” or separated from the remainder of the molecule, for example, to induce a pharmacological effect under specific physiological conditions. Thus, compounds of structure (I) are particularly useful as targeted therapeutic agents that may be systemically administered with minimal toxic side effects.

The desired number of repeating units and introduction of the desired linkers (“L^{a} groups”) into the molecule is accomplished by repeating the phosphoramidite coupling the desired number of times and by selecting the appropriate phosphoramidite monomer compound, for example, compounds of structure (e), provided above.

A representative, non-limiting example of a compound of structure (I) having a variety of different L^{a} groups and M groups is represented below by a compound of structure (f):

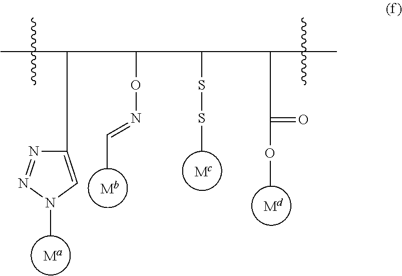 [SMILES: *C(C(=O)OC)C(SSC)C(O/N=C/C)C(*)c1cn(C)nn1]

wherein M^{a}-M^{d} each represent a different M moiety and the linkers comprise functional groups (tetrazole, oxime, disulfide and ester; left to right respectively) which have distinct methods of cleavage (Oxime—acidic hydrolysis; disulfide—reduction by TCEP (in vitro) or glutathione (in vivo); ester—esterase or base hydrolysis) or are non-cleavable (triazole).

Compounds of structure (I) can include multiple biologically active moieties (M groups), for example, M groups can be selected based on a complimentary or synergistic therapeutic strategy. In addition, embodiments of the present disclosure provide combinations of therapeutic agents, targeting moieties, and dye moieties (e.g., fluorophores) that can be used for simultaneous targeting, treatment, and detection. The ease of coupling polymer-drug constructs to targeting agents such as antibodies, antibody fragments, proteins, sugar moieties, receptor, receptor ligand, prion, aptamer, enzyme, or other clinically interesting agents provides utility to a wide variety of interesting applications (e.g., surface chemistries, assay development, etc.).Methods for preparing the compounds of structures (I) and/or (II) as well as methods for using automated DNA synthetic techniques for compound preparation are described in PCT Pub. No. WO 2015/027176, WO 2016/138461, and WO 2016/183185 all of which are incorporated herein in their entireties by reference.

The following examples are provided for purposes of illustration, not limitation.

### EXAMPLES

### General Methods

Mass spectral analysis was performed on a Waters/Micromass Quattro micro MS/MS system (in MS only mode) using MassLynx 4.1 acquisition software. Mobile phase used for LC/MS on dyes was 100 mM 1,1,1,3,3,3-hexafluoro-2-propanol (HFIP), 8.6 mM triethylamine (TEA), pH 8. Phosphoramidites and precursor molecules were also analyzed using a Waters Acquity UHPLC system with a 2.1 mm×50 mm Acquity BEH-C18 column held at 45° C., employing an acetonitrile/water mobile phase gradient. Molecular weights for monomer intermediates were obtained using tropylium cation infusion enhanced ionization on a Waters/Micromass Quattro micro MS/MS system (in MS only mode). Excitation and emission profiles experiments were recorded on a Cary Eclipse spectra photometer.

All reactions were carried out in oven dried glassware under a nitrogen atmosphere unless otherwise stated. Commercially available DNA synthesis reagents were purchased from Glen Research (Sterling, Va.). Anhydrous pyridine, toluene, dichloromethane, diisopropylethyl amine, triethylamine, acetic acid, pyridine, and THF were purchased from Aldrich. All other chemicals were purchase from Aldrich or TCI and were used as is with no additional purification.

### Example 1

### NHS Activation

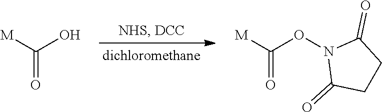

NHS activated M moieties are synthesized using standard coupling conditions. That is, a carboxy containing M moiety is dissolved in dichloromethane and to the mixture is added N,N′-dicyclohexylcarbodiimide (DCC) and N-hydroxysuccinimide (NHS). The final product is then purified as necessary and used in the next synthetic step. Alternatively, an M moiety can be modified to include a carboxy group, for example, using the synthetic strategy shown in Example 3 below.

### Example 2

### Monomer Synthesis

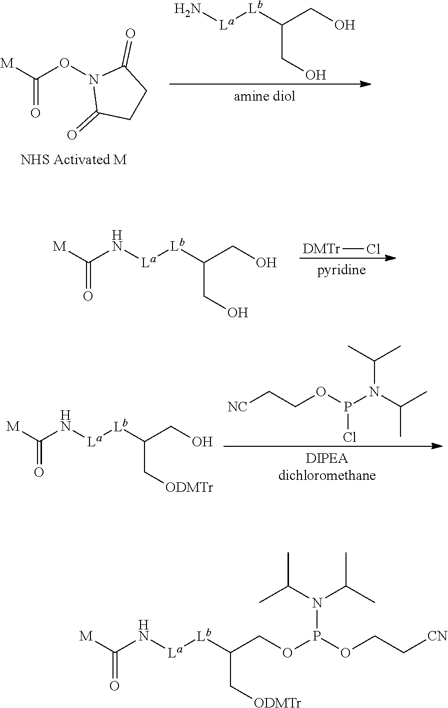

NHS activated M is reacted with an amine diol followed by the addition of a trityl protecting group to afford the trityl protected intermediate. The trityl protected intermediate is then reacted with 3-((chloro(diisopropylamino)phosphaneyl)oxy)propanenitrile to afford the final phosphoramidite product. The final phosphoramidite product is then used for automated DNA synthesis to incorporate a moiety comprising M (e.g., as a representative biologically active moiety or dye moiety) into embodiments of compounds of structure (I).

### Example 3

### Synthesis OF M-PEG-azide

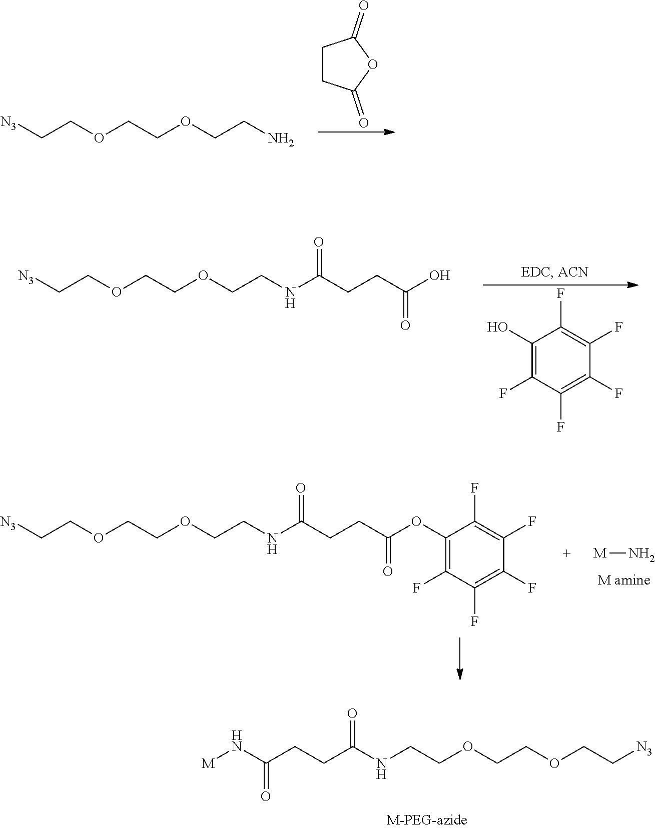

M-PEG-azide is synthesized according to the reaction sequence shown above. 2-(2-(2-azidoethoxy)ethoxy)ethan-1-amine is reacted with dihydrofuran-2,5-dione to afford intermediate 4-((2-(2-(2-azidoethoxy)ethoxy)ethyl)amino)-4-oxobutanoic acid. That intermediate is reacted with perfluorophenol to afford 4-((2-(2-(2-azidoethoxy)ethoxy)ethyl)amino)-4-oxobutanoate, which is coupled to M amine to afford the desired product, M-PEG-azide. The presence of the desired product is confirmed by LC-MS.

### Example 4

### Post Polymerization Modification 1

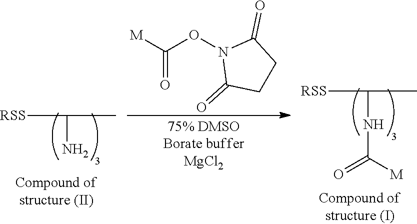

An exemplary compound of structure (II), having 3 pendant amine functional groups, is coupled with an NHS activated M moiety as shown in the reaction sequence above (note—for clarity, not all structural features of the compound of structure (II) are drawn). The reaction is carried out using a borate buffered H₂O/DMSO mixture (1:3) with magnesium chloride. The reaction successfully adds an M moiety to each of the 3 amine functional groups to afford a representative compound of structure (I) that is identified by LC-MS.

### Example 5

### Post Polymerization Modification 2

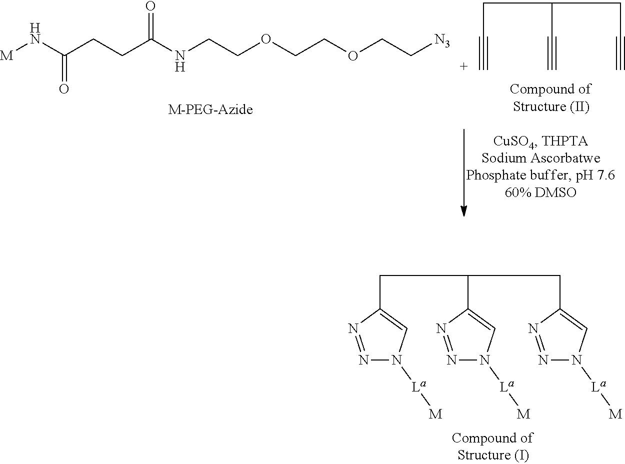

An exemplary compound of structure (II) having 3 pendant alkynyl functional groups is coupled to M-PEG-azide. Reaction conditions include CuSO₄, tris(3-hydroxypropyltriazolylmethyl)amine (THPTA), and sodium ascorbate. The reaction is carried out in phosphate buffered aqueous solvent with 60% DMS at a pH of 7.6. The reaction is run at room temperature and the presence of the desired product is confirmed by LC-MS. L^{a} represents heteroalkylene linker.

### Example 6

### Activation and Antibody Conjugation

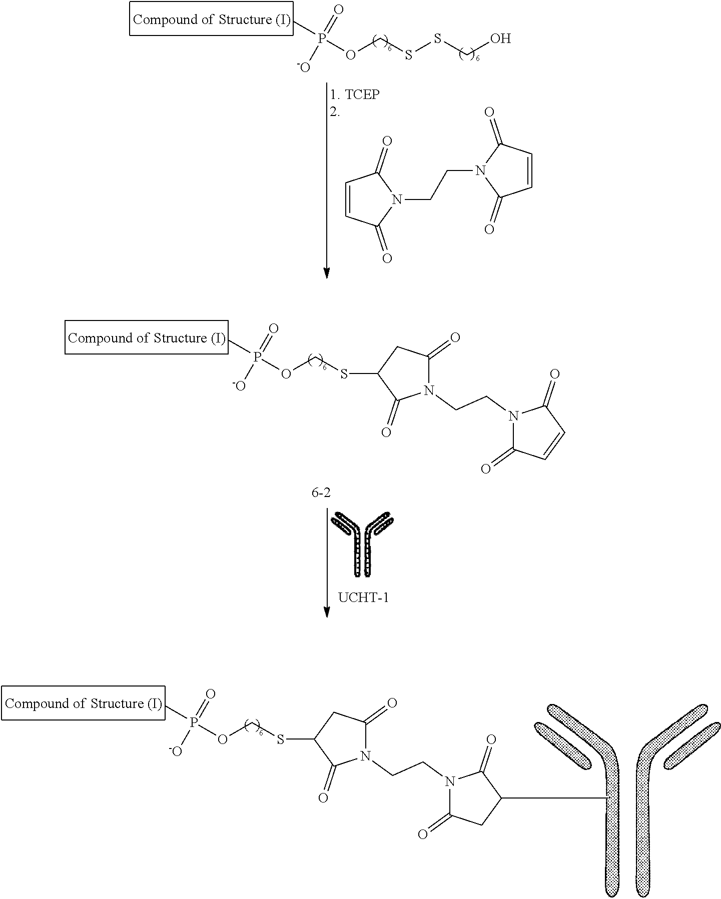

The thiol protecting group of a representative compound of structure (I) is removed using standard reducing conditions (i.e., TCEP) and the deprotected thiol is functionalized with 1,1′-(ethane-1,2-diyl)bis(1H-pyrrole-2,5-dione (bismaleimidoethane or “BMOE”) to afford 6-2. In parallel, an UCHT-1 antibody is treated with TCEP to reduce disulfide bonds. The reduced antibody is reacted with 6-2 (1.5 g) in a 5:1 molar ratio of polymer to antibody.

### Example 7

### Val-Cit-PABC (VCP) Phosphoramidite Monomer Synthesis

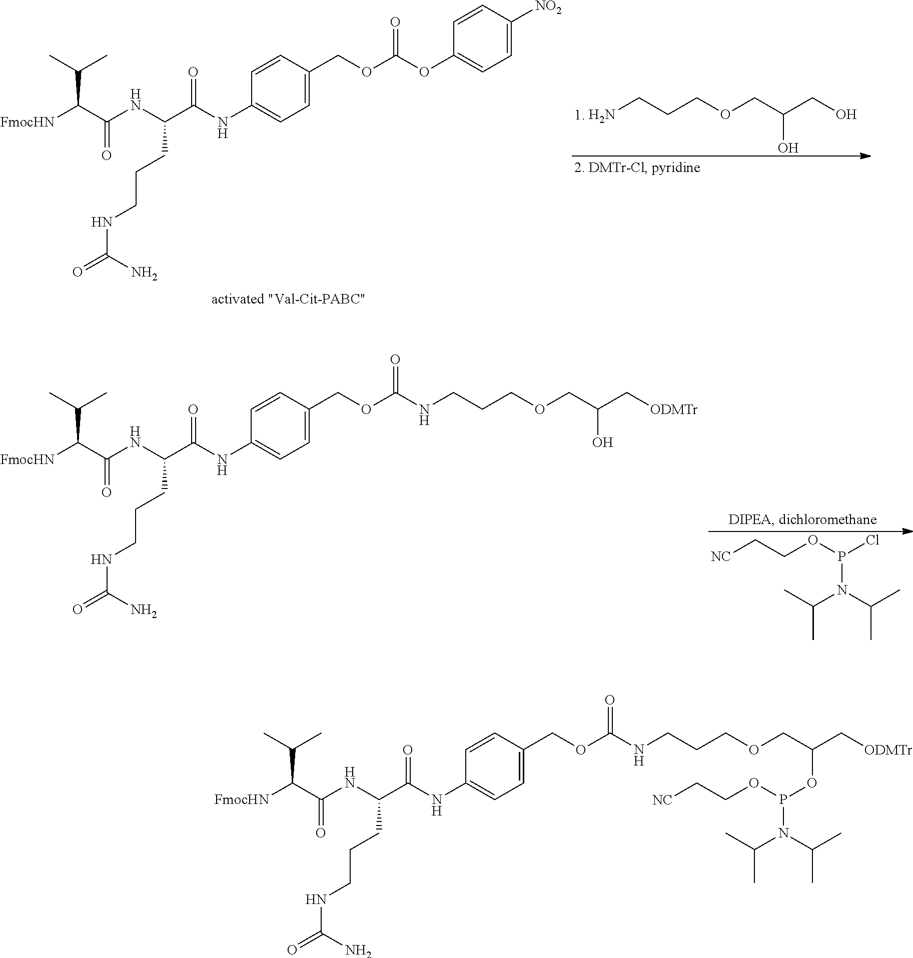

The 4-nitrophenol activated Val-Cit-PABC carbonate is reacted with 3-(3-aminopropoxy)propane-1,2-diol (Step 1) followed by the addition of a trityl protecting group to afford the trityl protected intermediate (Step 2). The trityl protected intermediate is then reacted with 3-((chloro(diisopropylamino)phosphaneyl)oxy)propanenitrile to afford the final phosphoramidite product. The final phosphoramidite product can then be used in a synthesis to afford compounds of structure (I).

### Example 8

### Pyridyl Disulfide Phosphoramidite Monomer Synthesis

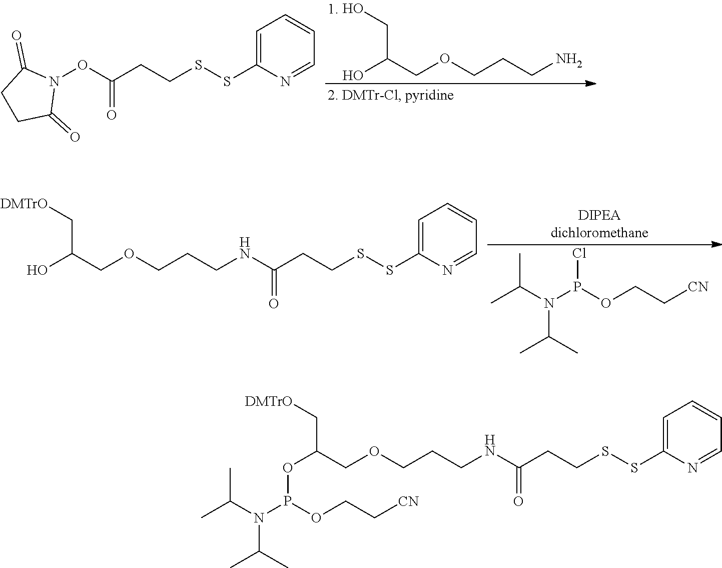

The N-hydroxysuccinimide activated pyridyl disulfide is reacted with 3-(3-aminopropoxy)propane-1,2-diol followed by the addition of a trityl protecting group to afford the trityl protected intermediate. The trityl protected intermediate is then reacted with 3-((chloro(diisopropylamino)phosphaneyl)oxy)propanenitrile to afford the final phosphoramidite product. The final phosphoramidite product can then be used in a synthesis to afford compounds of structure (I).

### Example 9

### Ethylamino Disulfide Phosphoramidite Monomer Synthesis

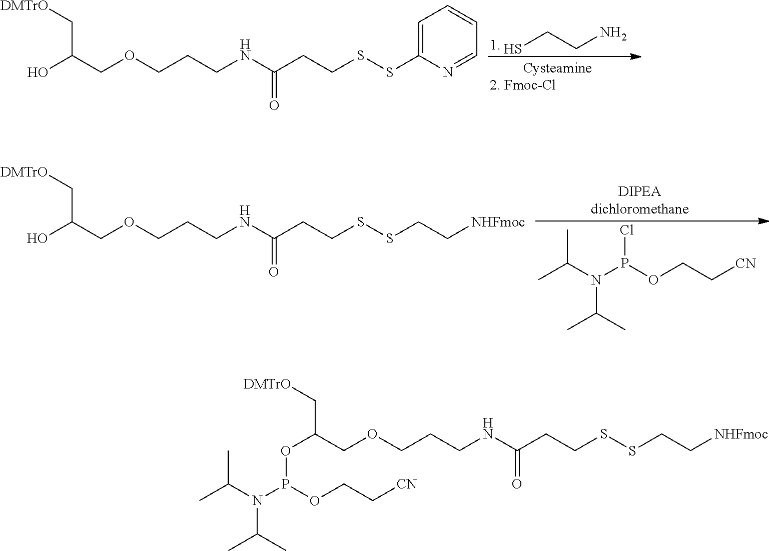

The trityl protected pyridyl disulfide from Example 8 is reacted with cysteamine followed by the addition of the Fmoc protecting group to afford the protected ethylamino disulfide intermediate. This intermediate is then reacted with 3-((chloro(diisopropylamino)phosphaneyl)oxy)propanenitrile to afford the final phosphoramidite product. The final phosphoramidite product can then be used in a synthesis to afford compounds of structure (I).

### Example 10

### N-hydroxyphthalimide Phosphoramidite Monomer Synthesis

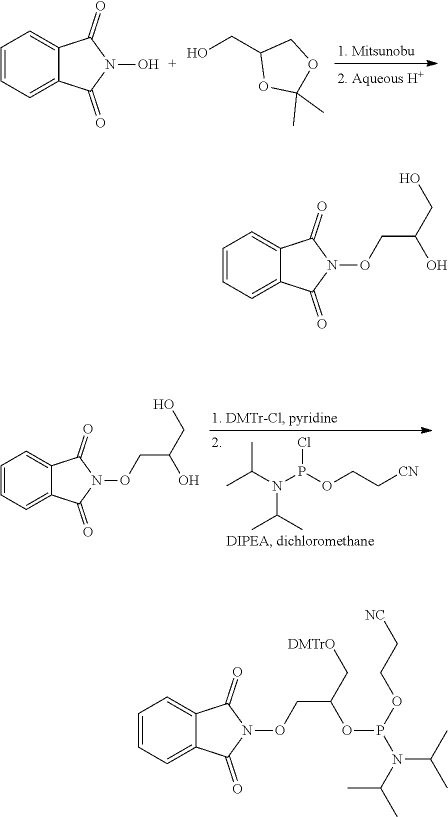

N-hydroxyphthalimide is reacted with (2,2-dimethyl-1,3-dioxolan-4-yl)methanol under Mitsunobu conditions, followed by deprotection under acidic conditions to provide the diol intermediate. The diol intermediate is then reacted with DMTr-Cl and pyridine to protect the primary alcohol as the trityl derivative. Subsequent reaction with 3-((chloro(diisopropylamino)phosphaneyl)oxy)propanenitrile affords the final phosphoramidite product. The final phosphoramidite product can then be used in a synthesis to afford compounds of structure (I).

### Example 11

### Synthesis of a Representative Compound of Structure (II) (Synthesis 1)

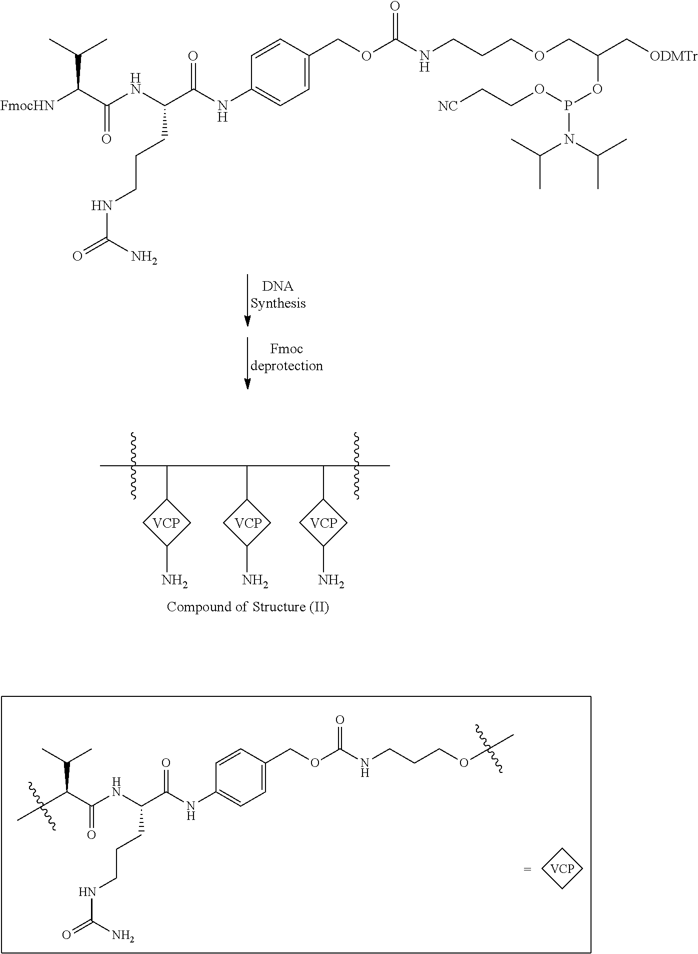

The Val-Cit-PABC phosphoramidite monomer described in Example 7 is reacted under appropriate conditions to afford an Fmoc protected polymer that undergoes a subsequent base promoted deprotection step with, for example, piperidine in DMF to provide a compound of structure (II). The amine functional groups can be coupled to an M moiety as described, for example, in Example 4 above (for clarity, not all structural features of the compound of structure (II) are drawn).

### Example 12

### Synthesis of a Representative Compound of Structure (II) (Synthesis 2)

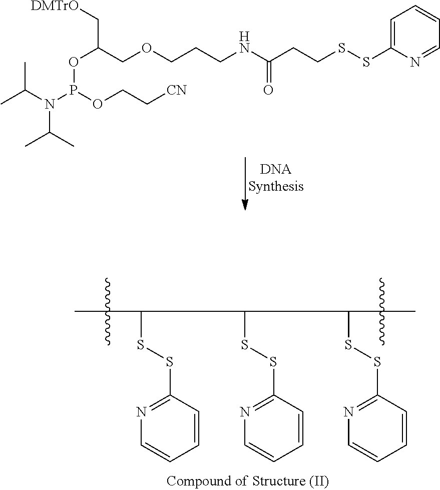

The pyridyl disulfide phosphoramidite monomer described in Example 8 is reacted under appropriate conditions to afford a pyridyl disulfide monomer. Reduction of this polymer, for example with TCEP, will cleave the disulfide, leaving a sulfhydryl group (—SH) that can be used for post synthetic introduction of an M moiety, through reaction with, for example, another sulfhydryl group or a maleimide group (for clarity, not all structural features of the compound of structure (II) are drawn).

### Example 13

### Synthesis of a Representative Compound of Structure (II) (Synthesis 3)

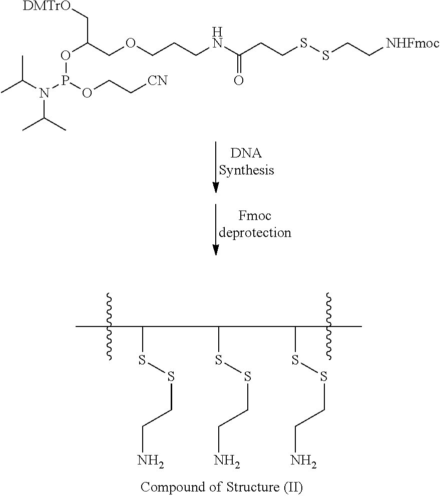

The ethylamino disulfide phosphoramidite monomer described in Example 9 is reacted under appropriate conditions to afford an N-Fmoc protected polymer that undergoes a subsequent base promoted deprotection step with, for example, piperidine in DMF, to provide a compound of the invention with an ethyl amine functionalized disulfide. The amine functional groups can be coupled to an M moiety as described, for example, in Example 4 above (for clarity, not all structural features of the compound of structure (II) are drawn).

### Example 14

### Synthesis of a Representative Compound of Structure (II) (Synthesis 4)

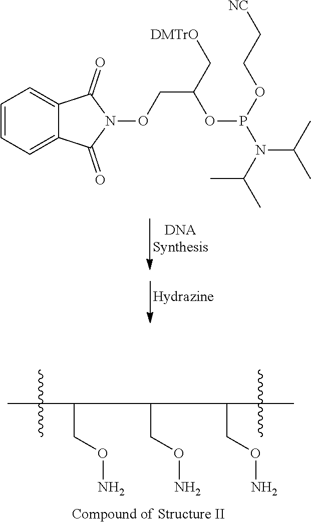

The N-hydroxyphthalimide phosphoramidite monomer described in Example 10 is reacted under appropriate conditions to afford a phthalimide polymer derivative that is reacted with hydrazine to form the alkoxy amine functional group. The alkoxy amine is reacted with a complementary group, for example an aldehyde or ketone, to form an oxime linkage to an M moiety (for clarity, not all structural features of the compound of structure (II) are drawn).

### Example 15

### Exemplary Synthesis of a Polymer of Structure (I)

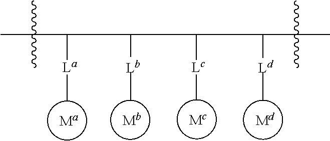

DNA synthesis methodology can be applied to build compounds of structure (I). Monomers (e.g., phosphoramidite monomers) can be purchased commercially (e.g., from ChemGenes Corporation, Wilmington Mass.) or synthesized using methods described herein (see, e.g., Examples 2 and 7-10). Introduction of M moieties is accomplished either during the DNA synthesis steps by including the M moiety as a portion of the monomer, or during a post-polymerization modification step (e.g., as described in Examples 4-6 and 11-14). An exemplary DNA synthesis scheme is shown below.

### A Representative DNA Synthesis Cycle

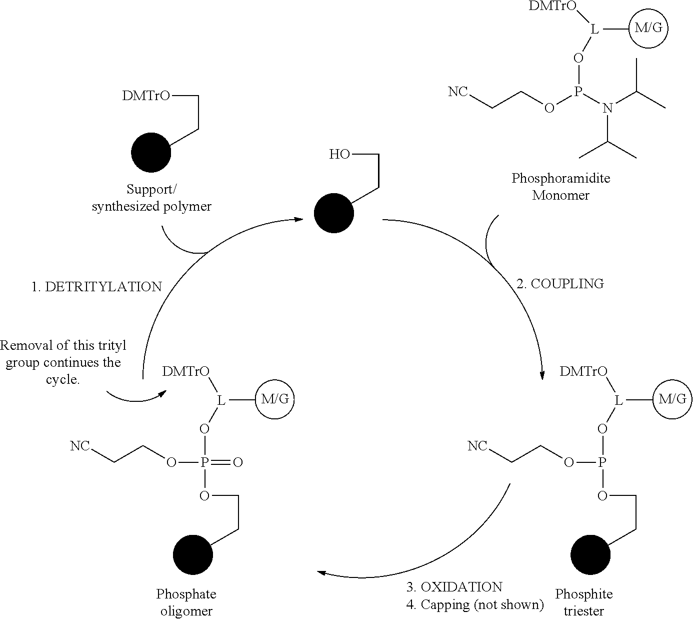

Oligomerization is initiated, typically, through the removal of a protecting group (e.g. a dimethoxytrityl group, DMTr) to reveal a free —OH (hydroxyl) group (Step 1, DETRITYLATION). In a subsequent coupling step, a phosphoramidite monomer is introduced that reacts with the free OH group making a new covalent bond to phosphorus, with concomitant loss of the diisopropyl amine group (Step 2, COUPLING). The resultant, phosphite triester is oxidized (e.g. with 12 and pyridine) to the more stable phosphate ester (Step 3, OXIDATION) and a capping step renders unreactive any remaining free OH groups (Step 4, CAPPING). The new product, phosphate oligomer, contains a DMTr protected OH group that can be deprotected to reinitiate the synthetic cycle so another phosphoramidite monomer can be appended to the oligomer.

Customization occurs at step 2 through the choice of phosphoramidite monomer. The nature of L, M and G are selected such that a desired compound of structure (I) is synthesized. M and G can be optionally absent to incorporate desired spacing between M and/or G moieties. A person of ordinary skill in the art can select multiple monomer types to arrive at compounds of the invention containing multiple therapeutic agents and/or other moieties (e.g., dyes) with concurrent variability in linker groups.

All of the U.S. patents, U.S. patent application publications, U.S. patent applications, foreign patents, foreign patent applications and non-patent publications referred to in this specification, including U.S. Provisional Patent Application Nos. 62/832,726, filed on Apr. 11, 2019, and 62/877,151, filed on Jul. 22, 2019, are incorporated herein by reference, in their entirety to the extent not inconsistent with the present description.

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

1. A compound having the following structure (I): 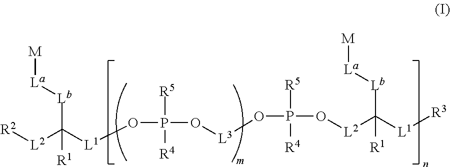
or a stereoisomer, pharmaceutically acceptable salt or tautomer thereof, wherein:
M is, at each occurrence, independently a biologically active moiety, or fragment thereof, a prodrug of a biologically active moiety, or fragment thereof, a fluorescent dye, an imaging agent, or a radioisotope binding site, provided at least one occurrence of M is not a fluorescent dye;
L^{a} is, at each occurrence, independently an optional physiologically cleavable linker and L^{b} is, at each occurrence, independently an optional physiologically non-cleavable linker, provided that at least one occurrence of L^{a} and L^{b} together comprise more than 4 carbons;
L¹ and L² are, at each occurrence, independently an optional alkylene, alkenylene, alkynylene, heteroalkylene, heteroalkenylene, heteroalkynylene or heteroatomic linker;
L³ is, at each occurrence, independently an alkylene, alkenylene, alkynylene, heteroalkylene, heteroalkenylene or heteroalkynylene linker;
R¹ is, at each occurrence, independently H, alkyl or alkoxy;
R² and R³ are each independently H, OH, SH, alkyl, alkoxy, alkylether, heteroalkyl, —OP(═Rₐ)(R_{b})R_{c}, Q, or a protected form thereof, or L′;
R⁴ is, at each occurrence, independently O⁻, S⁻, OZ, SZ or N(R⁶)₂, where Z is a cation and each R⁶ is independently H or alkyl;
R⁵ is, at each occurrence, independently oxo, thioxo or absent;
Rₐ is O or S;
R_{b} is OH, SH, O⁻, S⁻, ORₐ or SR_{d};
R_{c} is OH, SH, O⁻, S⁻, ORₐ, OL′, SR_{d}, alkyl, alkoxy, heteroalkyl, heteroalkoxy, alkylether, alkoxyalkylether, phosphate, thiophosphate, phosphoalkyl, thiophosphoalkyl, phosphoalkylether or thiophosphoalkylether;
R_{d} is a counter ion;
Q is, at each occurrence, independently a moiety comprising a reactive group, or protected form thereof, capable of forming a covalent bond with a complementary reactive group Q′ on a targeting moiety;
L′ is, at each occurrence, independently a linker comprising a covalent bond to Q, a targeting moiety, a linker comprising a covalent bond to a targeting moiety, a linker comprising a covalent bond to a solid support, a linker comprising a covalent bond to a solid support residue, a linker comprising a covalent bond to a nucleoside or a linker comprising a covalent bond to a further compound of structure (I);
m is, at each occurrence, independently an integer of zero or greater; and
n is an integer of one or greater.

2. (canceled)

3. The compound of claim 1 having the following structure (IA): 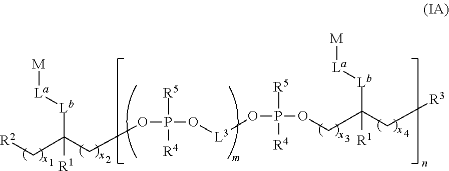
wherein:
x₁ and x₂ are each independently an integer from 0 to 10; and
x₃ and x₄ are, at each occurrence, independently an integer from 0 to 10.

4.-9. (canceled)

10. The compound of claim 2, wherein the compound has the following structure (IB): 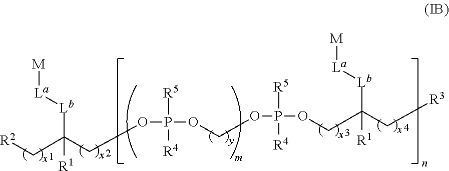
wherein:
x¹ and x² are each independently an integer from 0 to 6;
x³ and x⁴ are, at each occurrence, independently an integer from 0 to 6; and
y is an integer from 2 to 4.

11.-14. (canceled)

15. The compound of claim 1, wherein at least one occurrence of L^{a} comprises an amide bond, an ester bond, a phosphodiester bond, a disulfide bond, a double bond, a triple bond, an ether bond, a hydrazone, an amino acid sequence, a ketone, a diol, a cyano, a nitro or combinations thereof.

16. The compound of claim 15, wherein L^{a} comprises an amino acid sequence recognized by a sortase enzyme.

17. The compound of claim 16, wherein the amino acid sequence is Leu-Pro-X-Thr-Gly, wherein X is any amino acid residue.

18. (canceled)

19. (canceled)

20. The compound of claim 1, wherein at least one occurrence of L^{a} comprises one of the following structures:

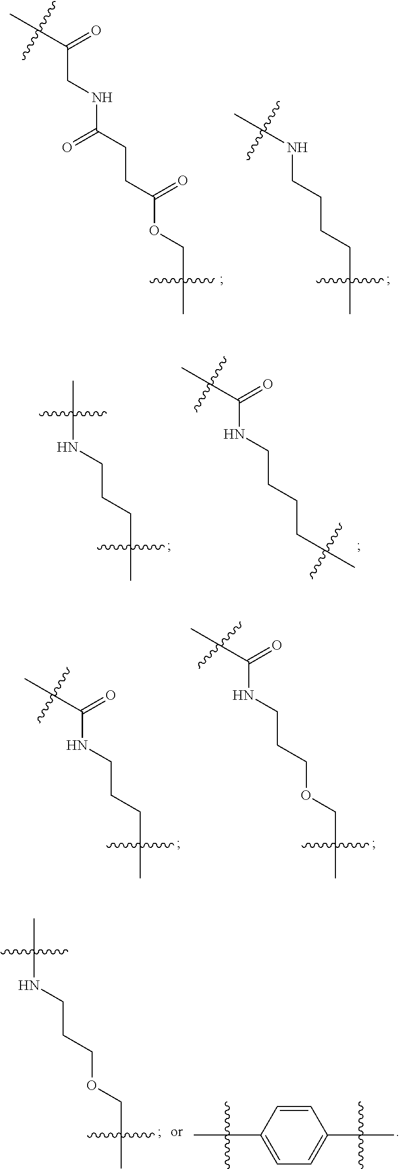

21.-27. (canceled)

28. The compound of claim 1, wherein at least one occurrence of L^{a} comprises one of the following structures:

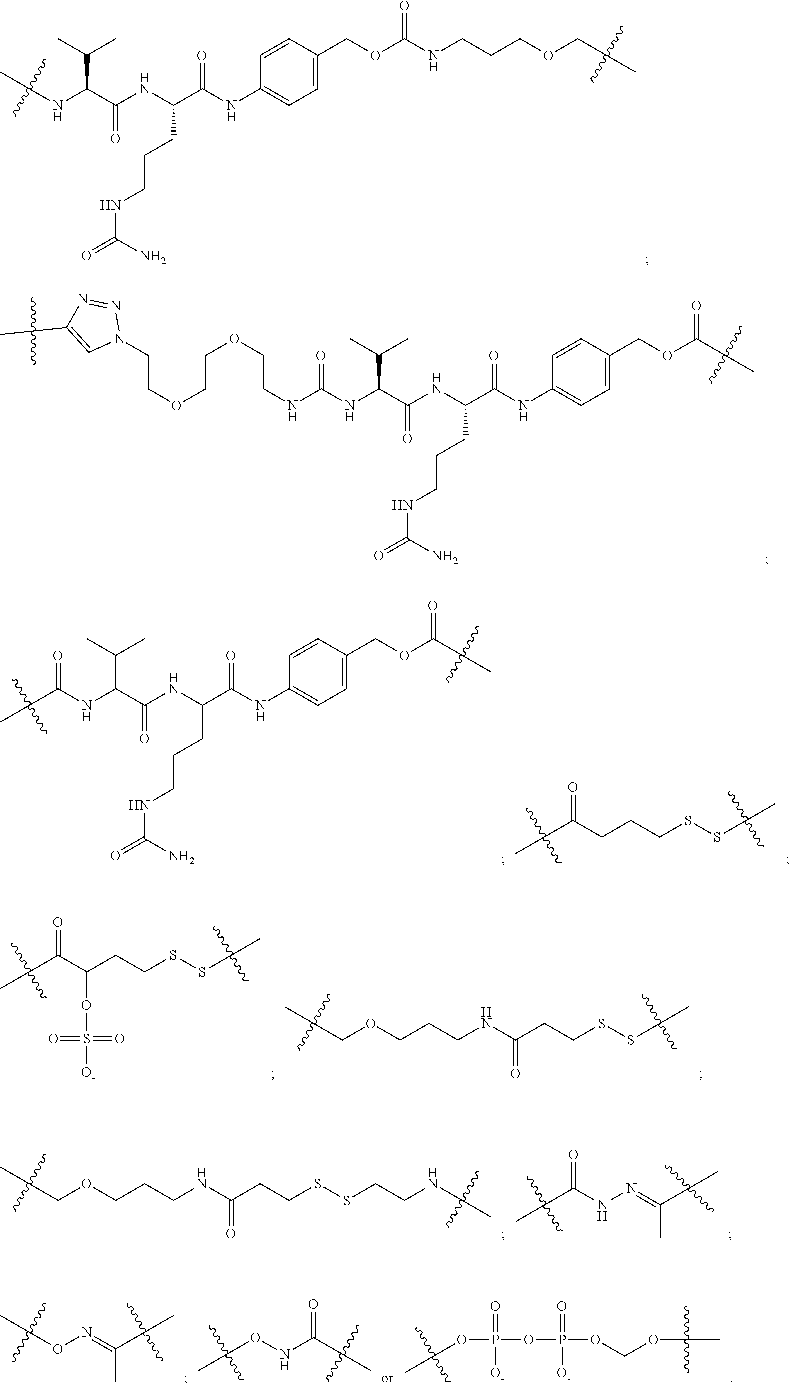

29.-34. (canceled)

35. The compound of claim 1, wherein at least one occurrence of L^{b} comprises a thioether bond.

36. (canceled)

37. The compound of claim 3, wherein at least one occurrence of L^{b} comprises one of the following structures:

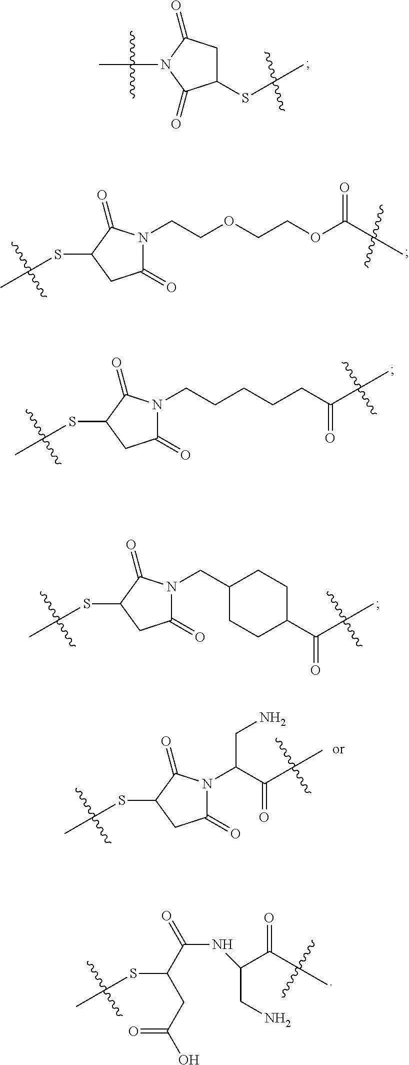 [SMILES: *SC1CC(=O)N(*)C1=O ; *SC1CC(=O)N(CCOCCOC(*)=O)C1=O ; *SC1CC(=O)N(CCCCCC(*)=O)C1=O ; *SC1CC(=O)N(CC2CCC(C(*)=O)CC2)C1=O ; *SC1CC(=O)N(C(CN)C(*)=O)C1=O ; *SC(CC(=O)O)C(=O)NC(CN)C(*)=O]

38.-50. (canceled)

51. The compound of claim 1, wherein L′ has the following structure: 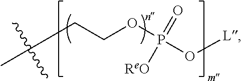
wherein:
m″ and n″ are independently an integer from 1 to 10;
R^{e} is H, an electron pair or a counter ion;
L″ is the targeting moiety or a linkage to the targeting moiety.

52. (canceled)

53. The compound of claim 1, wherein the targeting moiety is an antibody, cell surface receptor agonist, or cell surface receptor antagonist.

54. (canceled)

55. The compound of claim 53, wherein the targeting moiety is a monoclonal antibody, wherein the monoclonal antibody is Abciximab, Adalimumab, Alemtuzumab, Alirocumab, Avibactam, Basiliximab, Benralizumab, Bezlotoxumab, Blinatumomab, Brodalumab, Burosumab, Canakinumab, Caplacizumab, Certolizumab pegol, Daclizumab, Denosumab, Dupilumab, Eculizumab, Emicizumab, Erenumab, Evolocumab, Fremanezumab, Galcanezumab, Golimumab, Guselkumab, Ibalizumab, Idarucizumab, Infliximab, Itolizumab, Ixekizumab, Lanadelumab, Lokivetmab, Mepolizumab, Natalizumab, Obiltoxaximab, Ocrelizumab, Omalizumab, Palivizumab, Ranibizumab, Raxibacumab, Reslizumab, Rmab, Rovelizumab, Ruplizumab, Sarilumab, Secukinumab, Tildrakizumab, Thiomab, Tocilizumab, Ustekinumab, Vedolizumab, Abrilumab, Actoxumab, Aducanumab, Afasevikumab, Afelimomab, Anifrolumab, Anrukinzumab (IMA-638), Aselizumab, Atorolimumab, Bapineuzumab, BCD-100, Bertilimumab, Besilesomab, Biciromab, Bimagrumab, Bimekizumab, Birtamimab, Bleselumab, Blosozumab, Bococizumab, Brazikumab, Briakinumab, Brolucizumab, Carlumab, Carotuximab, Cedelizumab, Clazakizumab, Clenoliximab, Concizumab, Cosfroviximab, CR6261, Crenezumab, Crizanlizumab, Crotedumab, Depatuxizumab, mafodotin, Derlotuximab biotin, Dezamizumab, Diridavumab, Domagrozumab, Dusigitumab, Ecromeximab, Edobacomab, Efalizumab, Efungumab, Eldelumab, Elezanumab, Enokizumab, Eptinezumab, Erlizumab, Etrolizumab, Evinacumab, Exbivirumab, Fanolesomab, Faralimomab, Faricimab, Fasinumab, Felvizumab, Fezakinumab, Flanvotumab, Fletikumab, Flotetuzumab, Fontolizumab, Foravirumab, Frovocimab, Fulranumab, Gantenerumab, Gavilimomab, Gevokizumab, Gimsilumab, Gomiliximab, Gosuranemab, Ianalumab, Inclacumab, Inolimomab, Iomab-B, Keliximab, Lampalizumab, Landogrozumab, Larcaviximab, Lebrikizumab, Lenvervimab, Lerdelimumab, Letolizumab, Libivirumab, Ligelizumab, Lodelcizumab, Lulizumab pegol, Marstacimab, Mavrilimumab, Metelimumab, Mirikizumab, Motavizumab, Muromonab CD3, Nebacumab, Nemolizumab, NEOD001, Nirsevimab, Odulimomab, Olendalizumab, Olokizumab, OMS721, Opicinumab, Orticumab, Otelixizumab, Otilimab, Oxelumab, Ozanezumab, Ozoralizumab, Pagibaximab, Panobacumab, Pascolizumab, Pateclizumab, PDR001, Perakizumab, Pexelizumab, Placulumab, Plozalizumab, Ponezumab, Porgaviximab, Prasinezumab, Priliximab, PRO 140, Quilizumab, Rafivirumab, Ralpancizumab, Ranevetmab, Ravagalimab, Ravulizumab, Refanezumab, Regavirumab, Relatlimab, Rinucumab, Risankizumab, Roledumab, Romosozumab, Rontalizumab, SA237, Satralizumab, Sevirumab, SHP647, Sifalimumab, Simtuzumab, Siplizumab, Sirukumab, Solanezumab, Sonepcizumab, Spartalizumab, Stamulumab, Sulesomab, Suptavumab, Sutimlimab, Suvizumab, Suvratoxumab, Tadocizumab, Talizumab, Tamtuvetmab, Tanezumab, Tefibazumab, Telimomab aritox, Teneliximab, Teplizumab, Teprotumumab, Tezepelumab, Tibulizumab, Toralizumab, Tralokinumab, Trevogrumab, Tuvirumab, Ulocuplumab, Urtoxazumab, Varisacumab, Vepalimomab, Vesencumab, Visilizumab, Vobarilizumab, Zolimomab aritox, trastuzumab, gemtuzumab, brentuximab, vorsetuzumab, lorvotuzumab, cantuzumab, bivatuzumabor inotuzumab, or vadastuximab.

56. (canceled)

57. The compound of claim 1, wherein R² or R³ has one of the following structures:

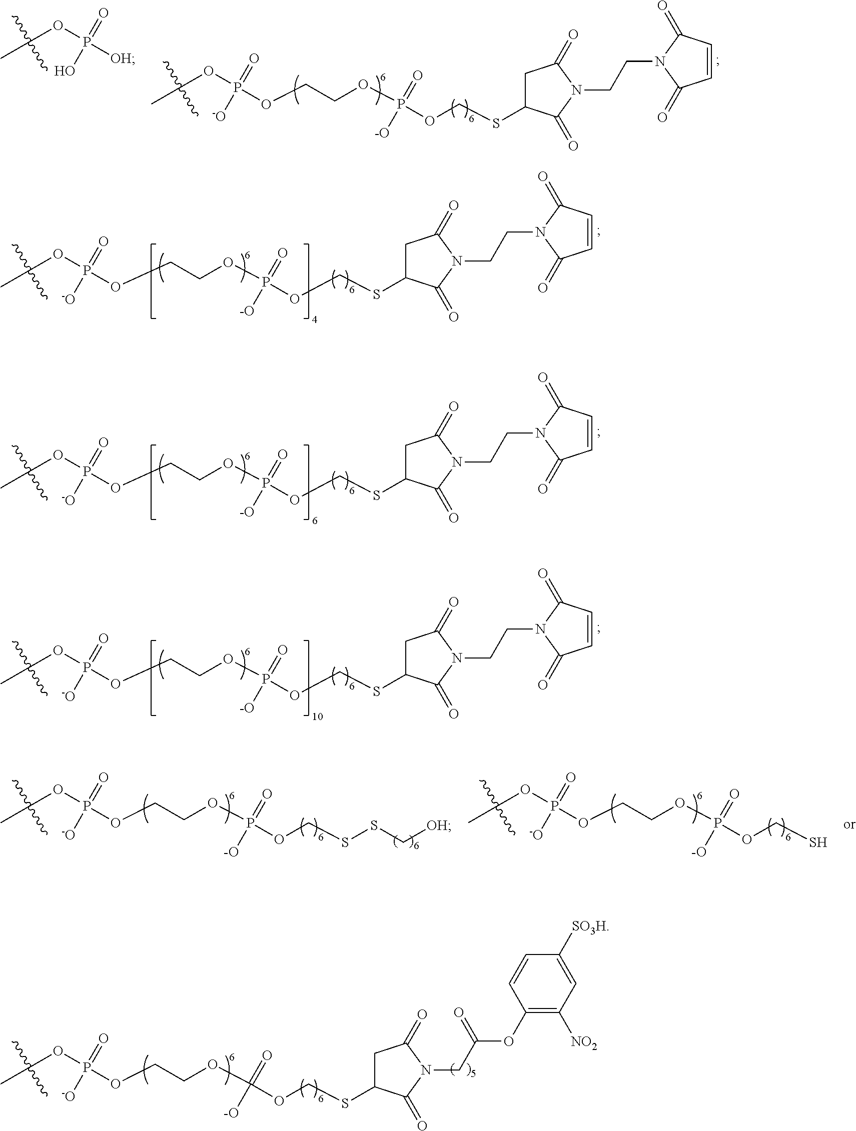

58. The compound of claim 1, wherein R³ has the following structure:

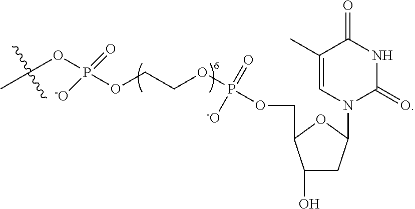

59.-70. (canceled)

71. The compound of claim 1, wherein M is, at each occurrence, independently an antineoplastic agent, an enediyne antitumor antibiotic, a maytansinoid, a topoisomerase inhibitor, a kinase inhibitor, an anthracycline, and EGFR inhibitor or an alkylating agent.

72. (canceled)

73. The compound of claim 1, wherein
A) at least one occurrence of M is selected from the group consisting of auristatin F, monomethyl auristatin F, monomethyl auristatin E, paciltaxol, SN-38, calicheamicin, anthramycin, abbeymycin, chicamycin, DC-81, mazethramycin, neothramycin A, neothramycin B, porothramycin prothracarcin, sibanomicin, sibiromycin, tomamycin, mertansine, emtansine, irinotecan, camptothecin, topotecan, silatecan, cositecan, Exatecan, Lurtotecan, gimatecan, Belotecan, and Rubitecan or
B) at least one occurrence of M has one of the following structures:

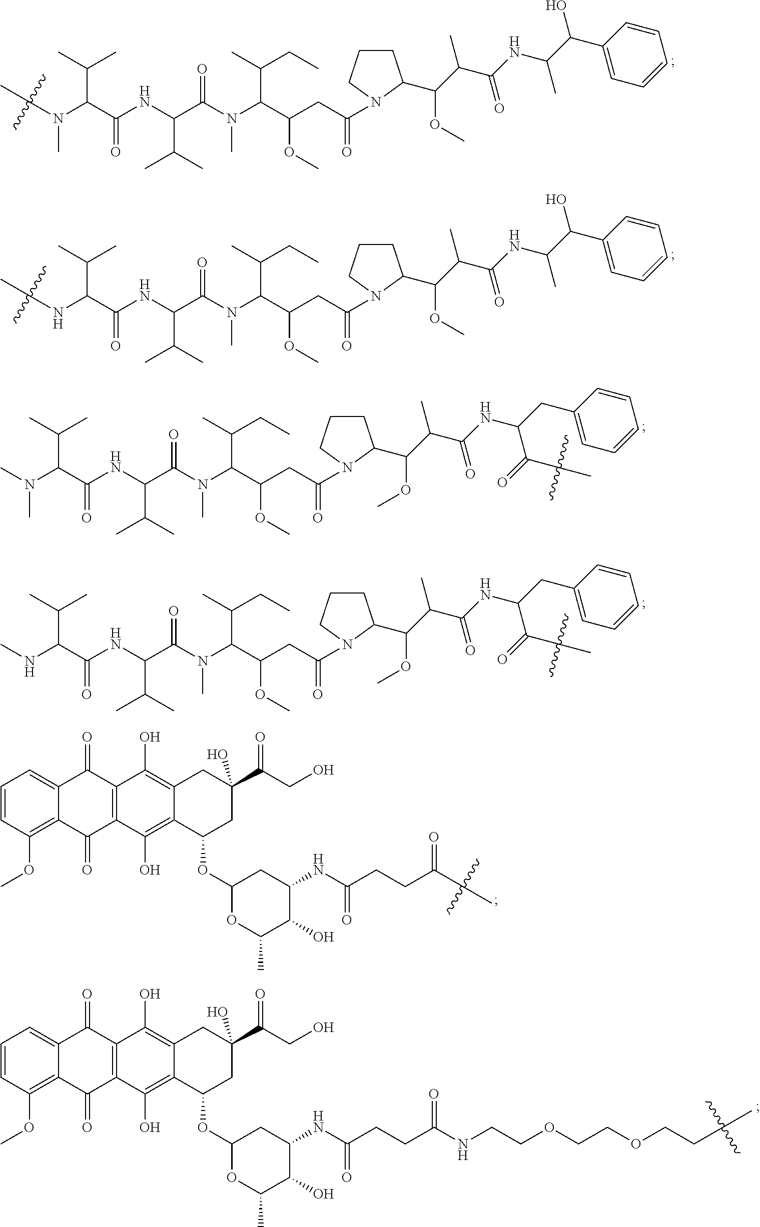

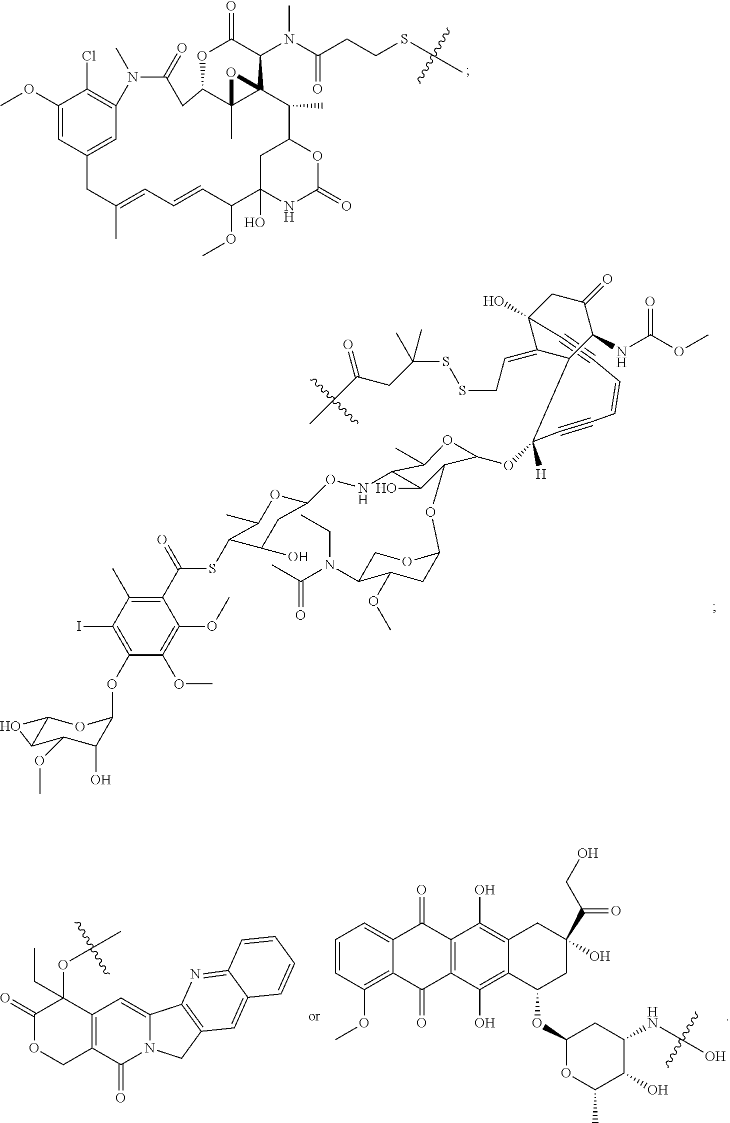

74. (canceled)

75. (canceled)

76. The compound of claim 1, wherein the compound has the following structure: 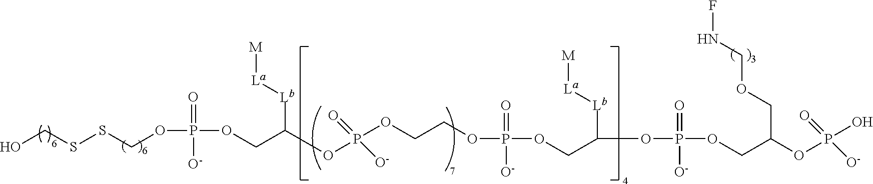
wherein
F has the following structure:

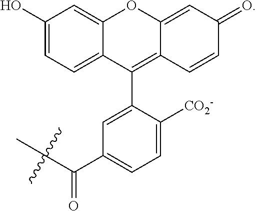

77. A composition comprising the compound of any one of claim 1 and a pharmaceutically acceptable carrier.

78. A method of treating a disease, the method comprising administering to a subject in need thereof a therapeutically effective amount of the composition of claim 77, wherein at least one M is a biologically active moiety effective for treating the disease.

79.-91. (canceled)
